(19) United States
(12) Patent Application Publication
(10) Pub. No.: US 2026/0275330 A1
(43) Pub. Date: Sep. 17, 2026

(54) METHODS AND COMPOSITIONS FOR PROTEIN ENGINEERING

(71) Applicant: PRIMROSE BIO, INC., San Diego, CA (US)

(72) Inventors: Helge ZIELER, Del Mar, CA (US); Dongxin Karen XU, San Diego, CA (US); Sabrina BAFFERT, San Diego, CA (US); Patrik ENGSTROM, San Diego, CA (US); Shawn WALSH, San Diego, CA (US)

(73) Assignee: PRIMROSE BIO, INC., San Diego, CA (US)

(21) Appl. No.: 19/471,959

(22) PCT Filed: Apr. 5, 2024
(86) PCT No.: PCT/US2024/023460

§ 371 Date: Oct. 2, 2025

Related U.S. Application Data
(60) Provisional application No. 63/494,338, filed on Apr. 5, 2023.

(51) Int. Cl. C12N 15/10; C12N 9/12; C12Q 1/48

(52) U.S. Cl. CPC C12N 15/1075; C12N 9/1247; C12Q 1/48

(57) ABSTRACT

The present disclosure provides compositions and methods for creating screening systems using RNA polymerase promoters and sequences encoding RNA polymerases for isolating genes encoding variant RNA polymerases with altered or improved properties related to in vitro transcription, or for isolating RNA polymerase promoter sequences, or for isolating untranslated sequences contributing to higher rates of protein synthesis from a nucleic acid molecule encoding a polypeptide or protein.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 4, 2024, is named “7003-0114PWO1.xml” and is 180,017 bytes in size. The sequence listing contained in this .XML file is part of the specification and is hereby incorporated by reference herein in its entirety.

### BACKGROUND

Enzymes, the catalysts of the biological world, are used for diverse applications in biotechnology, industry, food & feed, agriculture, and medicine. Most enzymes contained in man-made products or production systems are variants of those found in the natural world. Altering enzymes by mutation, fusion or other modification allows optimization of their activity for the intended application. Molecular evolution of enzymes is a specialized area in the larger field of protein engineering, a discipline that was pioneered in the 1980s and that has developed into a broad field employing many approaches and techniques, both empirical (laboratory-based) and computational, for optimizing the activity of a protein.

Many approaches and methods for protein engineering have been described in the literature, including but not limited to those listed in the following review articles: Leatherbarrow 1986, Zoller 1991, Lutz 2000, Leisola 2007, Eisenbeis 2010, O′Fágáin 2011, Foo 2012, Zawaira 2012, Marcheschi 2013, Woodley 2013, Johnson 2014, Packer 2015, Shin 2015, Chen 2016, Kaushik 2016, Swint-Kruse 2016, Wrenbeck 2017, Bornscheuer 2018, Lutz 2018, Singh 2018, Sinha 2019, Wilding 2019, and Yang 2019.

In general, protein engineering uses one or more methods to diversify the gene sequence encoding an enzyme of interest, followed by one or more selection or screening methods used to select genes that encode variant enzymes improved in one or more qualities of interest. Qualities of interest include, but are not limited to: catalytic efficiency; substrate specificity; resistance to inhibitors; stability when exposed to high temperature, or stability under reaction conditions that may contribute to loss of activity such as presence of solvents, salts or reaction products, or any other chemical or compound; high concentrations in the reaction of any of the aforementioned; or any other quality of the enzyme that may improve its suitability for an industrial process or a desired application.

Methods for diversifying a gene encoding an enzyme of interest include, but are not limited to: mutagenesis (i.e. introduction of point mutations); introduction of insertions and deletions of varying lengths within the enzyme coding sequence; fusion with other sequences either at the 5′ or the 3′ end of the coding sequence; homologous sequence exchange with related coding sequences resulting in reassortment of polymorphisms; and any other means of creating sequence diversity.

Methods and approaches used to select for genes encoding enzymes improved in one or more qualities of interest include approaches using in vitro compartmentalization in microdroplets or emulsions that allow efficient processing of high numbers of enzyme variants in small volumes. Such approaches have been described in the literature in a general manner and in specific applications to nucleic acid polymerases (Tawfik 1998, Ghadessy 2001, Ghadessy 2004, Diehl 2006, Griffiths 2006, Miller 2006, Ghadessy 2007, Zaher 2007, Tay 2010, Abil 2018, Sakatani 2019).

There are other examples of enzyme improvement via in vitro compartmentalization, such as ligases (Levy 2005, Paegel 2010, Lu 2014), phosphotriestarase (Griffiths, 2003), nucleases (Takeuchi 2014, Czapinska 2019, Zheng 2007, Bertschinger 2004, Doi 2004), proteases (Tran 2016, Tu 2011), methyltransferases (Griffiths 1998, Lee 2002, Cohen 2004), binding proteins (Fen 2007, Sepp 2005, Houlihan 2014, Chen 2008, Levy 2008), and others (Agresti 2005, Mastrobattista 2005, Bernath 2005, Aharoni 2005, Tay 2009, Stapleton 2010, Prodanovic 2011, Ostafe 2014, Ma 2014, Uyeda 2015, Ryckelynck 2015, Gianella 2016, Körfer 2022)

A critical advantage of in vitro compartmentalization is the fact that it allows many reactions to be conducted simultaneously without requiring each reaction to be set up separately in individual vessels or wells of a multiwell plate. The formation of distinct droplets creates the necessary compartmentalization to allow many reactions, occurring in individual droplets, to remain separated. This allows the simultaneous processing of many individual samples within a relatively small volume of a liquid mixture present in a single tube, vessel or container.

In vitro compartmentalization can be set up in a manner to allow for transcription of genes and translation of mRNAs to synthesize proteins within a compartmentalized sample, after formation of droplets. Emulsion-based screens can be set up with nucleic acid templates in a manner that every droplet in the emulsion contains on average a single molecule of template nucleic acid or a small number of nucleic acid template molecules. Co-encapsulation of RNA polymerases proteins, protein mixtures and/or cell-free extracts capable of transcription and translation allows proteins to be synthesized from genes encoded by the template molecules. If the population of template molecules contains variability in the template sequences, each droplet will sample a different sequence and allows for independent assessment of a particular feature of the sequence in question, in an ultra-high-throughput manner. The fundamental advantage of in vitro compartmentalization lies in its ability to link genotype to phenotype, and to do so on a miniaturized scale. While reactions set up in separate containers or separate wells in multi-well plates during high-throughput screening may allow screening of thousands or potentially tens of thousands of samples per day with the proper automation, in vitro compartmentalization greatly expands the possible number of simultaneous, individual reactions by several orders of magnitude to over one billion per sample.

To allow isolation of an improved version of a template nucleic acid or of a gene encoding a protein or nucleic acid of interest, in vitro compartmentalization often relies on creating a phenotype, a measurable or observable characteristic, that allows a selection or enrichment of compartments or droplets with that phenotype. In vitro compartmentalization strategies reported in the literature can rely on fluorescence-activated cell sorting (FACS) or other separation methods in which the phenotype of each compartment can be directly measured. However, compartment separation based on an observable or measurable phenotype is not always feasible or easy to do, and FACS sorting of water in oil emulsions to isolate individual droplets having particular characteristics, for example fluorescence, can be technically challenging.

Consequently, there remains a need for screening strategies and procedures, including with use of in vitro compartmentalization methods, that permit identification and isolation of desirable characteristics and/or phenotypes. The present disclosure describes such methods and their utility in discovering and developing sequences of interest to biotechnology, including sequences encoding improved RNA polymerases and non-coding sequences contributing to the expression of a gene of interest.

### BRIEF SUMMARY

As an alternative to designing a screen using in vitro compartmentalization in a manner that individual compartments acquire a phenotype, the present disclosure uses a novel method to enrich for nucleic acid templates encoding sequences with desirable characteristics. A self-expressed RNA polymerase gene, comprising a gene encoding an RNA polymerase and a promoter recognized by the same RNA polymerase placed upstream of the gene, allow enrichment of molecules encoding RNA polymerase variants, or variants of other sequences present in the template nucleic acid, by self-amplification during the reaction taking place in the emulsion or in vitro compartmentalized sample. This design can be used as the basis for a strategy to improve RNA polymerases for more efficient transcription of the nucleic acid template.

The present disclosure describes screening systems suitable for selecting RNA polymerases with improved activity. Because of the compact nature of in vitro compartmentalization, a screening system using in vitro compartmentalization is particularly well-suited for isolating genes encoding variant RNA polymerases with higher activity. To our knowledge, with the exception of ribozyme RNA polymerases (Zaher & Unrau, 2007) this is the only system described to date in which in vitro compartmentalization is used to select for higher-activity RNA polymerases.

The present disclosure also describes the use of dual promoters driving RNA polymerase gene expression that can be used to address a variety of interesting problems and enable discovery of useful improved enzymes and genetic sequences of biotechnological interest. For example, a dual-promoter system can be used to differentiate between transcriptional activity required to initiate transcription from a template in vitro and the subsequent transcriptional activity of RNA polymerases expressed from the initial transcript. Such a system relies on a DNA construct with a promoter specific to the RNA polymerase of interest, followed by a promoter for a different (and non-cross-reactive) initiating RNA polymerase, followed by a gene encoding the RNA polymerase of interest. Initiating RNA polymerase is added to reactions prior to the formation of emulsions, and it transcribes the RNA polymerase of interest, leading to expression. When that RNA polymerase of interest performs transcription, it binds to a site further upstream than the initiating RNA polymerase, and thereby creates a longer transcript. Subsequent steps can distinguish between these different transcripts.

The present disclosure describes compositions and methods for screening for improved enzymes using selection or enrichment systems employing nucleic acid templates containing dual promoters specific for nucleic acid polymerases, such as single-subunit RNA polymerases. Such dual-promoter templates have broad utility for optimizing nucleic acid polymerases and sequence elements found within nucleic acids that may alter nucleic acid stability, efficiency of transcription from nucleic acid templates or efficiency of translation (protein synthesis) from nucleic acid templates. Specifically, such selection or enrichment systems are suitable for use with in vitro compartments such as droplets of aqueous solution in a water-in-oil emulsion that allow rapid and efficient enrichment of genes encoding improved enzymes from a high number of gene variants in a small volume.

### BRIEF DESCRIPTION OF THE DRAWINGS

FIG. 1: Initiating RNA polymerase (1) added to the reaction recognizes its corresponding promoter (2) upstream of target RNA polymerase (3) and performs transcription (4) to create initiator transcript (5). Reaction components carry out in vitro expression (6) to produce target (7) RNA polymerase, which then recognizes its corresponding promoter (8) and transcribes (9) target transcript (10). A template-specific primer (11) is used during reverse transcription (12) to produce initiator cDNA (13) from initiator transcript and target cDNA (14) from target transcript. Primers that are specific to the longer target transcripts (15) are used in target-specific PCR amplification (16).

FIG. 2: Dual promoter design to enrich for superior 5′UTRs within in vitro compartments. The double-stranded DNA template contains two specific promoters and transcriptional start sites (pI and pT). Exogenously added initiating RNA polymerase (I-RNApol) transcribes the template DNA to synthesize the initiating transcript, from which the target RNA polymerase (T-RNApol) protein is expressed by translation machinery components present in the emulsion droplet. The target RNA polymerase (T-RNApol) in turn transcribes the DNA template to synthesize the target transcript. The amount of the target transcript reflects the efficiency of sequences present in the target transcript that contribute to translation of this transcript and synthesis of the target RNA polymerase. In this particular example, 5′ UTR sequences are randomized to allow isolation of novel, synthetic 5′ UTRs that efficiently direct translation to the ORF located downstream of the 5′ UTR. Use of in vitro compartmentalization with a water in oil emulsion separates different DNA template molecules into separate droplets and ensures that the target RNA polymerase acts on the specific 5′UTR variant template from which it was produced. Thus, the most efficient 5′UTRs are selected by multiple rounds of enrichment.

FIG. 3: A diversified template based on the PCR mutagenized template sequence given in SEQ ID NO:6 was subjected to multiple rounds of the in vitro screen described herein and then sequenced via next-generation sequencing to determine the frequency of amino acid substitutions present in the library. The maximum frequency of any amino acid substitution at each position of the RNA polymerase is shown for the starting library (dash-dot line), a single round of enrichment (dashed line), two iterative rounds of enrichment (dotted line), and three iterative rounds of enrichment (solid line). For each data series, positions were ranked in descending order based on the maximum amino acid substitution frequency at that position before plotting.

FIG. 4: A diversified template based on the PCR mutagenized template sequence given in SEQ ID NO:1 was subjected to multiple rounds of the in vitro screen described herein and then sequenced via next-generation sequencing to determine the frequency of amino acid substitutions present in the library. The maximum frequency of any amino acid substitution at each position of the RNA polymerase is shown for the starting library (dashed line), a single round of enrichment (dotted line), and two iterative rounds of enrichment (dotted line). For each data series, positions were ranked in descending order based on the maximum amino acid substitution frequency at that position before plotting.

FIG. 5: A diversified template based on the PCR mutagenized template sequence given in SEQ ID NO:2 was subjected to multiple rounds of the in vitro screen described herein and then sequenced via next-generation sequencing to determine the frequency of amino acid substitutions present in the library. The maximum frequency of any amino acid substitution at each position of the RNA polymerase is shown for the starting library (dotted line) and after the final round of enrichment (solid line). For each data series, positions were ranked in descending order based on the maximum amino acid substitution frequency at that position before plotting.

FIG. 6: A diversified template based on the PCR mutagenized template sequence given in SEQ ID NO:3 was subjected to multiple rounds of the in vitro screen described herein and then sequenced via next-generation sequencing to determine the frequency of amino acid substitutions present in the library. The maximum frequency of any amino acid substitution at each position of the RNA polymerase is shown for the starting library (dotted line) and after the final round of enrichment (solid line). For each data series, positions were ranked in descending order based on the maximum amino acid substitution frequency at that position before plotting.

FIG. 7: A diversified template based on the PCR mutagenized template sequence given in SEQ ID NO:4 was subjected to multiple rounds of the in vitro screen described herein and then sequenced via next-generation sequencing to determine the frequency of amino acid substitutions present in the library. The maximum frequency of any amino acid substitution at each position of the RNA polymerase is shown for the starting library (dotted line) and after the final round of enrichment (solid line). For each data series, positions were ranked in descending order based on the maximum amino acid substitution frequency at that position before plotting.

FIG. 8: A diversified template based on the PCR mutagenized template sequence given in SEQ ID NO:5 was subjected to multiple rounds of the in vitro screen described herein and then sequenced via next-generation sequencing to determine the frequency of amino acid substitutions present in the library. The maximum frequency of any amino acid substitution at each position of the RNA polymerase is shown for the starting library (dotted line) and after the final round of enrichment (solid line). For each data series, positions were ranked in descending order based on the maximum amino acid substitution frequency at that position before plotting.

### DETAILED DESCRIPTION

The following abbreviations and definitions are used for the interpretation of the specification and the claims.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus.

Unless expressly stated to the contrary, “or” refers to an inclusive “or” and not to an exclusive “or.” For example, a condition A or B is satisfied by any one of the following: A is true (or present) and B is false (or not present), A is false (or not present) and B is true (or present), and both A and B are true (or present). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Cap, 5′ cap or mRNA cap: As used herein, the terms “cap”, “5′ cap” and “mRNA cap” refer to specialized nucleotides found at the 5′ ends of mRNAs, such as the 7-methylguanosine cap found in eukaryotic mRNAs or other capping structures found at the 5′ end of natural or synthetic RNAs. mRNAs synthesized in vitro can be capped at their 5′ ends by co-transcriptional incorporation of dinucleotide or trinucleotide cap analogs by RNA polymerase.

Capping efficiency: As used herein, “capping efficiency” refers to the percentage of RNA synthesized in vitro using a single-subunit RNA polymerase that contains a 5′ cap.

Cap incorporation efficiency: As used herein, “cap incorporation efficiency” refers to the efficiency by which a single-subunit RNA polymerase incorporates a dinucleotide or trinucleotide cap analog into mRNA synthesized in vitro. An RNA polymerase with high cap incorporation efficiency can achieve higher capping efficiency at lower concentrations of dinucleotide or trinucleotide cap analog in the reaction than an RNA polymerase with lower cap incorporation efficiency.

Complementary nucleotide sequence: As used herein, a “complementary nucleotide sequence” is a polynucleotide sequence in which all of the bases are able to form base pairs with another polynucleotide sequence of the opposite 5′ to 3′ polarity, such that all bases in each polynucleotide chain are paired with their counterpart, forming base pairs.

Control elements: As used herein, “control elements” refers to nucleotide sequences located upstream (5′ non-coding sequences), within, or downstream (3′ non-coding sequences) of a coding sequence and which influence the transcription, RNA processing or stability, or translation of the associated coding sequence. Regulatory sequences include, but are not limited to, promoters, translation leader sequences, introns, polyadenylation recognition sequences, RNA processing sites, effector binding sites, and stem-loop structures.

Degenerate Sequences: As used herein, a “degenerate sequences” are defined as populations of sequences where specific sequence positions differ between different molecules or clones in the population. The sequence differences may be a single nucleotide or multiple nucleotides of any number, examples being 2, 3, 4, 5, 6, 7, 8, 9, 10, 20, 30, 40, 50, 60, 70, 80, 90, 100, 200, 300, 400, 500, 600, 700, 800, 900, 1000 nucleotides, or any number in between. Sequence differences in a degenerate sequence may involve the presence of 2, 3 or 4 different nucleotides in that position within the population of sequences, molecules or clones. Examples of degenerate nucleotides in a specific position of a sequence are: A or C; A or G; A or T; C or G; C or T; G or T; A, C or G; A, C or T; A, G or T; C, G or T; A, C, G or T.

Diversified sequence: As used herein, a “diversified sequence” refers to a nucleic acid sequence which has been derived from a parental nucleic acid sequence and altered to create one or more mutant or variant versions of the parental sequence. Alterations can be by mutagenesis (i.e. introduction of point mutations); introduction of insertions and deletions of varying lengths; sequence randomization (i.e. by replacement of one or more sequence stretches within the parental sequence with degenerate sequences or the same length or of different lengths); fusion with other sequences either at the 5′ or the 3′ end of the parental sequence; homologous sequence exchange with related or homologous sequences resulting in reassortment of polymorphisms; or combinations thereof; and any other means of creating sequence diversity. Diversified sequences are often created as populations of sequence variants, where a single nucleic acid sample contains nucleic acid molecules related to each other by sequence but differing in their specific sequence.

DNA: DNA is a nucleic acid that is a polymer of deoxyribonucleotides. DNA occurs in single stranded or double stranded forms. As used herein, DNA contains nucleotide residues each of which has a 2′ carbon in the form CH₂.

Expression: As used herein, “expression” refers to the transcription and stable accumulation of sense (mRNA) or antisense RNA derived from the nucleic acid disclosed, as well as the accumulation of polypeptide as a product of translation of mRNA.

Fidelity: As used herein, Fidelity describes the accuracy of a nucleic acid polymerase, reflecting faithful copying of a template nucleic acid into a daughter nucleic acid strand. Fidelity also describes the accuracy by which a nucleic acid sample reflects the sequence of the template nucleic acid from which it was copied. For example, a high fidelity DNA or RNA polymerase makes few errors in copying a DNA strand and results in a DNA or RNA sample that is substantially free of misincorporated nucleotides that change the sequence from that of the template DNA when copied into an RNA or a DNA daughter strand. A high fidelity RNA sample is one that contains few misincorporated nucleotides that change the sequence from that of the template DNA from which the RNA sample was derived.

Free nucleotide: As used herein, ‘free nucleotide means a monomeric nucleotide, typically in solution.

Frequency rank: As used herein, “frequency rank” refers to data generated via next-generation sequencing. After the “maximum amino acid substitution frequency” is calculated for each position in an enzyme sequence, those positions are then sorted in descending order (i.e. ranked) by that frequency. The resulting ranks begin at 1 and end at a numerical value equal to the total number of positions in the enzyme sequence.

Full-length Open Reading Frame: As used herein, a “full-length open reading frame” refers to an open reading frame encoding a full-length protein which extends from its natural initiation codon to its natural final amino-acid coding codon, as expressed in a cell or organism. In cases where a particular open reading frame sequence gives rise to multiple distinct full-length proteins expressed within a cell or an organism, each open reading frame within this sequence, encoding one of the multiple distinct proteins, is considered full-length. A full-length open reading frame can either be continuous or interrupted by introns.

Full-length Protein: As used herein, a “full-length protein” is a polypeptide which extends from its natural first amino acid to its natural final amino acid, as encoded in the genome of a cell or organism and expressed in the cell or organism.

Full-length RNA or full-length transcript: “Full-length RNA” or “full-length transcript” as used herein refers to an RNA synthesized from a nucleic acid template that covers the entire length of the nucleic acid template, from the transcription initiation site in a 3′ to 5′ direction along the template strand to the end of the nucleic acid template. An RNA molecule transcribed from a nucleic acid template may be considered full-length if it is substantially full-length, meaning that its length differs from the length of the nucleic acid template by a few or multiple nucleotides at either end, such that the migration of a full-length RNA molecule and the substantially full-length RNA molecule cannot be distinguished using commonly used methods of gel electrophoresis and capillary gel electrophoresis. Full-length RNA can also be alternatively referred to as “target RNA”.

Gene: As used herein, “gene” refers to a nucleic acid fragment that is capable of being expressed as a specific protein, optionally including regulatory sequences preceding (5′ non-coding sequences) and following (3′ non-coding sequences) the coding sequence. “Native gene” or “natural gene” refers to a gene as found in nature in its natural host organism, complete with its natural control sequences including but not limited to a promoter, terminator, ribosome binding site or other translation promoting sequence, enhancer, and repressor binding sites. “Chimeric gene” refers to any gene that comprises regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. Similarly, a “foreign” gene refers to a gene not normally found in the host organism, but that is introduced into the host organism by gene transfer. Foreign genes include native genes inserted into a non-native organism, or chimeric genes. A “transgene” is a gene that has been introduced into the genome by a transformation procedure.

In-Frame: As used herein, “in-frame” and particularly in the phrase “in-frame fusion polynucleotide,” refers to the reading frame of codons in an upstream or 5′ polynucleotide or open reading frame (ORF) as being the same reading frame as the reading frame of codons in a polynucleotide or ORF placed downstream or 3′ of the upstream polynucleotide or ORF that is fused with the upstream or 5′ polynucleotide or ORF. Such in-frame fusion polynucleotides encode a fusion protein or fusion peptide encoded by both the 5′ polynucleotide and the 3′ polynucleotide.

In vitro transcription (IVT) reaction: As used herein, “in vitro transcription reaction” or “IVT reaction” is a reaction designed to produce RNA by transcribing a DNA template in vitro. In vitro transcription reactions contain one or more DNA template molecules encoding the RNAs to be transcribed; one or more completely or partially purified single-subunit RNA polymerases; nucleoside triphosphates as substrates for the single-subunit RNA polymerase(s) such as the four canonical ribonucleoside triphosphates ATP, CTP, GTP and TTP; buffers, divalent cations and salts as necessary for the reaction. IVT reactions can also contain additional enzymes such as a pyrophosphatase that degrades pyrophosphate released by the RNA polymerase during RNA synthesis. The nucleic acid template contains a promoter sequence recognized by the RNApol and where the RNApol binds to initiate the transcript.

Integrity of a nucleic acid or RNA integrity: “Integrity of a nucleic acid” or “RNA integrity” as used herein, refers to the degree to which a collection of nucleic acid molecules have the expected length. For example, RNA molecules transcribed from a linear double-stranded DNA template that measures 2000 base pairs between the transcription start site and the end of the template (measured along the template strand and including the transcription start site) are expected to have a length of 2000 nucleotides. When measured by gel electrophoresis or capillary gel electrophoresis, such RNA molecules may range in size from 250 nucleotides to 2000 nucleotides. If, as measured by gel electrophoresis or capillary gel electrophoresis, half of the RNA molecules have the expected length of 2000 nucleotides and the other half are shorter, then the integrity of this RNA sample is 50%, or stated differently the sample has RNA integrity of 50%. The portion of the RNA molecules which, as measured by gel electrophoresis or capillary gel electrophoresis, have a length of approximately 2000 base pairs corresponds to full-length and substantially full-length RNA molecules.

“In vitro translation reaction” as used herein is a cell-free reaction designed to produce a protein by translating and RNA transcript in vitro. In vitro translation reactions contain one or more RNA transcripts to be translated, ribosomes, initiation and elongation factors, tRNAs charged with amino acids, ATP, and optionally accessory proteins to enhance protein folding.

Iterate/Iterative: As used herein, to “iterate” means to apply a method or procedure repeatedly to a material or sample. Typically, the processed, altered or modified material or sample produced from each round of processing, alteration or modification is then used as the starting material for the next round of processing, alteration or modification. “Iterative” selection refers to a selection process that iterates or repeats the selection two or more times, using the survivors of one round of selection as starting material for the subsequent rounds.

Library: As used herein, a “library” of genes or polynucleotide sequences is a collection of sequences that are different from each other and that are cloned into a vector for propagation of the sequences. In different libraries, the sequences differ by sequence content, origin, source organism, length, structure, association with other sequences, and/or any other property of a polynucleotide sequence. For example, a library of amino acid repeat fusion genes is generated by cloning a starting open reading frame (ORF) collection that contains multiple different ORFs encoded by the E. coli genome into a bacterial cloning and expression vector that contains a promoter, a sequence encoding an amino acid repeat oriented in a manner that this sequence will be joined directly and in-frame to the ORFs, a terminator, a plasmid backbone and an antibiotic resistance gene. The starting ORF collection can contain any number of ORFs that number 5 or greater, for example 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 or greater, or any number in between. In a specific aspect of the disclosure, the ORF collection used to generate the library contains a sufficient number of ORFs to give a high likelihood of encoding a specific desirable property of E. coli, for example 50% or more of the ORFs encoded by the E. coli genome, or 2074 or more ORFs when using the annotation of the E. coli strain MG1655 genome annotation prepared by the University of Wisconsin, Madison which lists a total of 4148 ORFs.

Linker sequence: As used herein, “linker sequence” refers to a polynucleotide sequence or polypeptide sequence separating two polynucleotides or polypeptides in a fusion polynucleotide or fusion polypeptide. For example, a fusion polynucleotide contains two or more open reading frames (ORFs) that are separated by a linker sequence, which encodes a peptide which separates the two parts of the polypeptide that results from expression and translation of the fusion polynucleotide. A linker can also separate an epitope tag from a protein or enzyme. Linker sequences can have diverse length and/or sequence composition.

Maximum amino acid substitution frequency: As used herein, “maximum amino acid substitution frequency” refers to data generated via the analysis of next-generation DNA sequencing (NGS) results. For a particular sample, the NGS sequence reads are mapped to the reference gene and nucleic acid substitutions and their corresponding amino acid substitutions are determined. For all observed amino acid substitutions occurring at each amino acid position of the reference gene, the amino acid substitution frequency is determined as the number of reads containing the observed amino acid substitution divided by the total number of reads mapping to that position. The maximum amino acid substitution frequency is then defined as the maximum frequency of any amino acid substitution for a given position.

Non-homologous: As used herein, “non-homologous” is defined as having sequence identity at the nucleotide level of less than 50%.

Nucleic acid: As used herein, “nucleic acid” refers to biopolymers, consisting of nucleotides joined to each other via phosphodiester linkages, phosphorothioate linkages or other linkages. “Nucleic acid” or “Nucleic acid molecule” can be used interchangeably with “polynucleotide.” As used herein, “nucleic acid” can also refer to a single strand of nucleic acid. A nucleic acid can either consist of deoxyribonucleotide residues, in which case it is DNA, or ribonucleotide residues, in which case it is RNA, or it can contain both deoxyribonucleotide residues and ribonucleotide residues in which case it is a chimeric nucleic acid.

Nucleic Acid Template or Template Nucleic Acid Molecule: As used herein, “nucleic acid template” or “template nucleic acid molecule” is a nucleic acid molecule present in an in vitro transcription reaction in that serves as the template for synthesis of a homologous nucleic acid with a nucleic acid polymerase. For example, a double-stranded DNA template containing a specific promoter for a single-subunit RNA polymerase serves as the nucleic acid template for an RNA molecule homologous to the sense strand of the nucleic acid template. The nucleic acid template is often simply referred to as the “template.”

Nucleic Acid Polymerase: As used herein, “nucleic acid polymerase” refers to an enzyme that catalyzes the polymerization of a nucleic acid using nucleoside triphosphates and unblocked nucleic acids as substrates and sequentially adds single nucleotides to the 3′ end of the unblocked nucleic acid. Nucleic acid polymerases as described in the scientific literature typically fall into the classes of DNA polymerases and RNA polymerases, with DNA polymerases capable of polymerizing DNA and RNA polymerases capable of polymerizing RNA. However, specific enzymes may have the dual ability to catalyze the synthesis of both DNA and RNA. For example, a DNA polymerase may have the ability to add ribonucleotides to the 3′ end of a DNA or RNA molecule, and an RNA polymerase may have the ability to add deoxyribonucleotides to the 3′ end of a DNA or RNA molecule.

Nucleic acid synthesis: As used herein, “nucleic acid synthesis” is the process by which nucleic acids are produced in nature or by man, minimally requiring a nucleic acid polymerase, one or more nucleoside triphosphates as monomer building blocks, and a nucleic acid substrate.

De novo nucleic acid synthesis: As used herein, “de novo nucleic acid synthesis” refers to synthesis of man-made DNA, involving controlled addition of specific nucleotides to a nucleic acid substrate to create a specific sequence and structure of nucleic acid.

Nucleotides: As used herein, “nucleotides” are the monomer building blocks of nucleic acids, made of three components: a 5-carbon sugar, a phosphate group and a nitrogenous base. The two main classes of nucleotides are deoxyribonucleotides, the building blocks of DNA and ribonucleotides, the building blocks of RNA. If the sugar is ribose, the nucleic acid is RNA; if the sugar is the ribose derivative deoxyribose, the nucleic acid is DNA. As used herein, a deoxyribonucleotide has the group CH₂ as the 2′ carbon in the ribose sugar. All other structures of the 2′ carbon are grouped under the term ribonucleotides. As used herein, a nucleotide can mean a nucleotide residue present within a nucleic acid, a nucleoside monophosphate, a nucleoside diphosphate, a nucleoside triphosphate or any derivative or modification thereof.

Nucleoside triphosphates: As used herein, “nucleoside triphosphates” are defined as any of the ribonucleoside triphosphates ATP, CTP, GTP, ITP, UTP and XTP, etc. used in RNA synthesis, or any of the deoxyribonucleoside triphosphates dATP, dCTP, dGTP, dITP, dTTP and dXTP, etc. used in DNA synthesis, or any modified analogs, derivatives or variants thereof, including derivatives containing phosphorothioate linkages. Mixtures of the four canonical nucleoside triphosphates used in DNA synthesis (dATP, dCTP, dGTP, and dTTP) are denoted by the shorthand “dNTP” and mixtures of the four canonical nucleoside triphosphates used in RNA synthesis (ATP, CTP, GTP, and UTP) are denoted by the shorthand “NTP”.

Oligonucleotide: As used herein, “oligonucleotide” refers to a single stranded nucleic acid consisting of two or more nucleotides.

Open Reading Frame (ORF): As used herein, an “open reading frame (ORF)” is defined as any sequence of nucleotides in a nucleic acid that encodes a protein or peptide as a string of codons in a specific reading frame. Within this specific reading frame, an ORF can contain any codon specifying an amino acid, but does not contain a stop codon. The ORFs in a starting collection need not start or end with any particular amino acid. An ORF is either continuous or is interrupted by one or more introns.

Operably linked: As used herein, “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

Peptide bond: As used herein, a “peptide bond” is a covalent bond between a first amino acid and a second amino acid in which the alpha-amino group of the first amino acid is bonded to the alpha-carboxyl group of the second amino acid.

Percentage of sequence identity: As used herein, “percent sequence identity” refers to the degree of identity between any given query sequence, e.g. SEQ ID NO: 10, and a subject sequence. A subject sequence typically has a length that is from about 80 percent to 200 percent of the length of the query sequence, e.g., 80, 81, 82, 83, 84, 85, 86, 87, 88, 89, 90, 91, 93, 95, 97, 99, 100, 105, 110, 115, 120, 130, 140, 150, 160, 170, 180, 190 or 200 percent of the length of the query sequence or any number in between. A percent identity for any subject nucleic acid or polypeptide relative to a query nucleic acid or polypeptide is determined as follows. A query sequence (e.g. a nucleic acid or amino acid sequence) is aligned to one or more subject nucleic acid or amino acid sequences using the computer program ClustalW (version 1.83, default parameters), which allows alignments of nucleic acid or protein sequences to be carried out across their entire length (global alignment, Chenna 2003).

Protein coding sequence: The term protein coding sequence in this disclosure is used synonymously with “open reading frame” and refers to a nucleic acid sequence that encodes a polypeptide or protein.

To determine a percent identity of a subject or nucleic acid or amino acid sequence to a query sequence, the sequences are aligned using Clustal W, the number of identical matches in the alignment is divided by the query length, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher website and at the European Bioinformatics Institute website on the World Wide Web. Sequence identity can be 5%, 6%, 7%, 8%, 9%, 10%, 11%, 12%, 13%, 14%, 15%, 16%, 17%, 18%, 19%, 20%, 30%, 40%, 50%, 60%, 70%, 75%, 80%, 85%, 90%, 91%, 92%, 93%, 94%, 95%, 96%, 97%, 98%, 99%, and any percentage value in between.

Plasmid and Vector: The terms “plasmid” and “vector” refer to genetic elements used for carrying genes which are not a natural part of a cell or an organism. Plasmids typically replicate extrachromosomally as autonomous episomal genetic elements, while vectors can either integrate into the genome or can be maintained extrachromosomally as linear or circular DNA fragments. Plasmids and vectors can be linear or circular, and can consist of single- and/or double-stranded DNA or RNA that is derived from any source. Plasmids and vectors often contain a number of nucleotide sequences from different sources which have been joined or recombined into a unique construction which is useful for introducing polynucleotide sequences into a cell or an organism and expressing genes within an organism. The sequences present on a plasmid or on a vector include but are not limited to: autonomously replicating sequences; centromere sequences; genome integrating sequences; origins of replication; control sequences such as promoters and/or terminators; open reading frames; selectable marker genes such as antibiotic resistance genes; visible marker genes such as genes encoding fluorescent proteins; restriction endonuclease recognition sites; recombination sites; and/or sequences with no apparent or known function.

Polypeptide or protein: as used herein, the terms “polypeptide” or “protein” denote a polymer composed of a plurality of amino acid monomers joined by peptide bonds. The polymer comprises 10 or more monomers, including 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000 or any number in between.

Promoter: As used herein, “promoter” refers to a DNA sequence capable of controlling the expression of a coding sequence or functional RNA. In general, a coding sequence is located 3′ to a promoter sequence. Promoters can be derived in their entirety from a native gene, and/or can be composed of different elements derived from different promoters found in nature, or even comprise synthetic DNA segments. It is understood by those skilled in the art that different promoters direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

Random/Randomized: As used herein, “random” or “randomized” means made or chosen without method or conscious decision.

Randomized sequence: As used herein, ‘randomized sequence’ refers to a nucleic acid sequence in which one or more nucleotides have been replaced by degenerate nucleotides.

RNA: As used herein, “RNA” is a nucleic acid that is a polymer of ribonucleotides. RNA occurs in single stranded or double stranded forms. As used herein, RNA contains nucleotide residues each of which has a 2′ carbon in a form other than CH₂.

RNA polymerase/RNApol/RNAP as used herein refers to an enzyme that synthesizes a single-stranded RNA molecule from a nucleic acid template, usually double-stranded DNA.

RNA quality: “RNA quality” as used herein refers to the purity of RNA obtained in an in vitro transcription reaction. High RNA quality can mean high RNA integrity, high capping efficiency, low levels of double-stranded RNA, low levels of short, truncated RNAs, low levels of other undesirable side products other than the full-length RNA, high fidelity, high and/or uniform polyA tail length, or any combinations thereof. Low RNA quality can mean low RNA integrity, low capping efficiency, high levels of double-stranded RNA, high levels of short, truncated RNAs, high levels of other undesirable side products, low RNA fidelity, low and/or non-uniform polyA tail length, or any combinations thereof. High RNA quality typically results in high rates of translation of the RNA into the functional or active protein encoded by the RNA.

Sequence: As used herein, “sequence,” when used in a biological context, can imply the sequence of nucleotides in a nucleic acid or the sequence of amino acids in a protein. As used herein, the term “sequence” has a meaning dependent on the context in which the term is used. For example, when used in the context suggesting nucleic acids such as genome sequences, gene sequences or ORFs, then “sequence” refers to a nucleotide sequence. In a context suggesting proteins or polypeptides, such as the proteome, proteins or enzymes, “sequence” refers to amino acid sequence.

Single-subunit RNA polymerase: “Single-subunit RNA polymerase” as used herein, refers to an enzyme with DNA-dependent RNA polymerase activity capable of synthesizing RNA from a DNA template in vitro in a pure form, without the presence or addition of any other proteins or peptides into the reaction.

Template-independent Nucleic Acid Synthesis: As used herein, “template-independent nucleic acid synthesis” is a process by which a nucleic acid polymerase catalyzes the polymerization of a nucleic acid without use of a template strand that is base paired to the nucleic acid being synthesized and that serves as the template for the strand being synthesized.

Transcriptional 5′ end: the term “transcriptional 5′ end” refers to the first ribonucleotide in an RNA transcript. Transcripts generated by single-subunit RNA polymerases contain triphosphates at their transcriptional 5′ ends (Hornung 2006).

Transformed: As used herein, “transformed” means genetic modification by introduction of a polynucleotide sequence.

Transformation: As used herein, “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

Transformed Organism: As used herein, a “transformed organism” is an organism that has been genetically altered by introduction of a polynucleotide sequence into the organism's genome.

Unfavorable Conditions: As used herein, “unfavorable conditions” implies any part of the growth condition, physical or chemical, that results in slower growth than under normal growth conditions, or that reduces the viability of cells compared to normal growth conditions.

Untranslated sequence: As used herein, “untranslated sequence” refers to untranslated regions (or UTRs) that occur on both sides (5′ and 3′) of a protein-coding sequence in a nucleic acid sequence. If it is found on the 5′ or leading side of the ORF or protein-coding sequence, it is called the 5′ UTR; if it is found on the 3′ side of the ORF or protein-coding sequence, it is called the 3′ UTR. The term “untranslated sequence” refers to sequences present within mRNA which is transcribed from a corresponding DNA sequence and then translated into protein. Several regions of the mRNA, including 5′ UTRs, 3′ UTRs and polyA tails are untranslated sequences because they are usually not translated into protein.

Variant nucleic acids: As used herein, variant nucleic acid refers to mutated or altered versions of nucleic acid sequences. A variant nucleic acid may have point mutations, insertions, deletions, inversions, rearrangements or combinations thereof compared to the parental or reference sequence that it is derived from or related to. Sequences within a variant nucleic acid that contain mutations, insertions, deletions, inversions, rearrangements or combinations thereof compared to a reference or parental sequence that the variant nucleic acid is related to or derived from, may be of any length, including 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 nucleotides or more, or any number in between. A variant nucleic acid may contain a single change (single mutation, insertion, etc.) compared to a reference or parental sequence, or multiple changes. A variant nucleic acid may have uniform sequences represented within a sample, where all molecules in a nucleic acid sample have the same sequence, or diverse sequences where a sample comprises nucleic acid molecules of different sequence. Variant nucleic acids comprising nucleic acid molecules of different sequence may differ from each other in sequence positions anywhere in the nucleic acid. Variant nucleic acids comprising nucleic acid molecules of different sequence may differ from each other in a particular region of the sequence, or have differences scattered over the entire length of the sequence, or combinations thereof. Variant nucleic acids can contain degenerate or randomized positions, where a specific sequence or region has been replaced by a stretch of degenerate nucleotides. Randomized or degenerate positions within variant nucleic acids may involve adjacent nucleotides or non-adjacent nucleotides separated by nucleotides of a specific or fixed sequence. Variant nucleic acids are frequently employed in biotechnology to create variability within a sequence of interest (coding sequence or non-coding sequence) from which new nucleic acids with specific qualities of interest (for example higher efficiency of an encoded enzyme) can be isolated.

Variant proteins or variant enzymes: As used herein, a “variant protein” or “variant enzyme” refers to a protein or enzyme which is related to but distinct from a parental protein or enzyme by alteration of the parental amino acid sequence, resulting in one or more mutant or variant versions of the parental protein or enzyme. Alterations can be by mutagenesis (i.e. introduction of single amino acid changes); introduction of insertions and deletions of varying lengths; fusion with other sequences either at the N or the C-terminus of the parental protein; sequence exchange with related proteins resulting in hybrid or chimeric proteins containing blocks of sequence from two or more parental proteins; or combinations thereof; and any other means of creating sequence diversity. Variant proteins or enzymes are often created as populations of sequence variants, where a single protein sample contains protein molecules related to each other by sequence but differing in their specific sequence.

Different RNA polymerases differ in their ability to synthesize RNA. RNA synthesis by an RNA polymerase can also be influenced by the reaction components of the in vitro transcription reaction. For example, the ability of a single-subunit RNA polymerase to synthesize a uniform population of RNA molecules in vitro decreases with the length of the DNA molecule used as a template for the RNA polymerase. Certain RNA polymerases are capable of synthesizing RNAs of 100 nucleotides, 500 nucleotides, 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 40 kb, 50 kb in length or longer or shorter, or any length in between. Certain RNA polymerases have higher processivity than others, or an improved ability to synthesize full-length RNAs from longer templates (for example, templates encoding mRNAs longer than 5 kb), and are capable of synthesizing highly uniform RNAs greater than 1 kb in length or longer. The composition of the in vitro transcription reaction, including the concentrations of the main reaction components (double-stranded DNA template molecules encoding the RNAs to be transcribed; single-subunit RNA polymerases; nucleotide triphosphates as monomers for RNA synthesis; buffers, divalent cations and salts as necessary for the RNApol to be active and accessory enzymes such as pyrophosphatase).

Single-subunit RNA polymerases and/or in vitro transcription reactions also differ in their ability to utilize non-natural nucleotides and incorporate these into the RNA molecule. Examples of such non-natural nucleotides are 2′-O-methyl NTPs, 2′-fluoro NTPs, pseudouridine-5′-triphosphate and N1-methylpseudouridine-5′-Triphosphate. The 2′ hydroxyl of ribonucleotides has frequently been targeted for modification because this group is primarily responsible for the low stability of RNA under basic conditions. Various modifications at the 2′ position of nucleotides have been tested for increasing RNA stability. However, some single-subunit RNA polymerases incorporate such modified nucleotides inefficiently. Alternatively, RNA molecules containing such modified nucleotides may exhibit a high rate of sequence errors. Specific single-subunit RNA polymerases among the ones described in this disclosure are able to incorporate modified nucleotides efficiently without compromising sequence fidelity.

Single-subunit RNA polymerases and/or in vitro transcription reactions differ in their RNA yield based on the nucleotides added to an in vitro transcription reaction. For example, a 1 ml in vitro transcription reaction containing 5 mM of each of the four nucleotide triphosphates ATP, CTP, GTP and TTP can yield up to about 6.43 mg of RNA (the ‘theoretical yield’) assuming equal representation of each of the nucleotides in the DNA template and complete incorporation of nucleotide triphosphates into RNA in the reaction. An RNA polymerase that synthesizes 2.5 mg of RNA in such a reaction has a yield of 38.9%. Higher-yielding RNA polymerases and/or in vitro transcription reactions are of value as they maximize the amount of RNA product made from a specific amount of nucleotide triphosphates added to the reaction. For example, the accessory proteins disclosed herein increase the transcript yield generated by T7 RNA polymerase or by RNApol137.

Yield enhancement during in vitro transcription can mean increasing the absolute amount of RNA synthesized in the reaction with all reaction components being the same (approaching the theoretical yield) or increasing RNA yield while reducing the reaction concentrations of the double-stranded DNA template or of the RNA polymerase. Such improved reactions are said to increase RNA yield on template or on RNA polymerase. Accessory proteins added to an in vitro transcription reaction can increase the RNA yield on template or increase the RNA yield on RNA polymerase or increase the RNA yield on any other reaction component that is expensive or otherwise limiting and for which it may benefit the producer of the RNA to lower the concentration of said component.

RNA yield as described above can be expressed as total RNA yield, which includes all RNA molecules synthesized in the reaction, regardless of their length, or full-length RNA yield, which includes only the full-length and substantially full-length RNA molecules synthesized in the reaction. For example, an RNA polymerase or in vitro transcription reaction may produce a measurably higher RNA yield than full-length RNA yield. Addition of certain accessory proteins to in vitro transcription reaction may change either total RNA yield or full-length RNA yield.

Single-subunit RNA polymerases and/or in vitro transcription reactions differ in the amount of double-stranded RNA made in a reaction. Double-stranded RNA is a frequent and undesirable side product of in vitro transcription reactions (Arnaud-Barbe 1998, Mu 2018, Gholamalipour 2018), and its reduction or elimination reduces the cost of synthesizing pharmaceutical-grade RNA.

Single-subunit RNA polymerases and/or in vitro transcription reactions differ in the amount of short or truncated RNAs made in a reaction. Short or truncated RNAs can be any RNAs that are not full-length and are frequent and undesirable side products of in vitro transcription reactions. They represent aborted or incomplete transcription products of a template (Martin 1988); their reduction or elimination reduces the cost of synthesizing pharmaceutical-grade RNA.

Single-subunit RNA polymerases differ in their ability to synthesize polyA sequences encoded in DNA templates. RNAs synthesized with RNA polymerases that don't efficiently synthesize polyA sequences may have truncated polyA sequences present in the transcribed RNA, or the polyA sequences present in the RNA may be of diverse length. For example, the ability to efficiently synthesize poly A sequences longer than 50 nucleotides, or to synthesize these in a uniform manner, with equal or near-equal length of the polyA sequence in each synthesized RNA molecule, is of great interest to the use of RNAs in biotechnology.

Single-subunit RNA polymerases and/or in vitro transcription reactions differ in their ability to incorporate a 5′-cap such as the 7-methylguanosine cap found in eukaryotic mRNAs or other capping structures into the 5′ end of RNAs. mRNAs used in biotechnology can be capped by incorporating a specialized dinucleotide or trinucleotide cap analog into the 5′ end of the mRNA. Co-transcriptional incorporation of dinucleotide or trinucleotide caps is catalyzed by the RNA polymerase during transcription initiation. The composition of in vitro transcription reactions as disclosed herein can be varied to increase the rate of cap incorporation and cap utilization.

Single-subunit RNA polymerases and/or in vitro transcription reactions differ in their temperature specificity or reaction speed at varying temperatures, both of which are important parameters in RNA synthesis. Lower reaction temperatures such as between 10° C. and 20° C. can stabilize the RNA. However, T7 RNA polymerase has very low activity at such temperatures. It is therefore of value to identify RNA polymerases active at low temperatures.

Single-subunit RNA polymerases differ in their stability at different temperatures. For certain applications it may be useful to develop RNA polymerase with increased stability at temperatures at which T7 RNA polymerase shows loss of activity.

Single-subunit RNA polymerases and/or in vitro transcription reactions differ in their overall reaction speed, irrespective of temperatures. Faster enzymes are typically more desirable because shorter reaction times reduce RNA degradation.

Single-subunit RNA polymerases and/or in vitro transcription reactions differ in their fidelity. High-fidelity RNA polymerases will produce RNAs that faithfully encode the sequence of the template DNA used to synthesize the RNA and faithfully encode a protein of interest. High-fidelity RNA polymerases therefore have higher utility when synthesizing RNAs for therapeutic or vaccine applications.

Measurements of RNA polymerase activity, or quality metrics of RNA synthesized in in vitro transcription reactions, are generated using standardized methods and assays. RNA yield is measured by purification of the RNA after the in vitro transcription reaction, followed by spectroscopic or fluorescence measurement of RNA concentrations (Gandhi 2020, Hadi 2023). RNA yield and integrity are measured by gel electrophoresis (Henderson 2021, Tu 2024) and quantitation of the fluorescence intensity of RNA bands using ImageJ or related software (Schindelin 2012, Schneider 2012, Rueden 2017, Poveda 2019) or other methods for quantitating fluorescent band intensities. RNA yield and integrity are also determined with capillary electrophoresis-based methods (Poveda 2019, Warzak 2023) using commercially available instruments such as the Fragment Analyzer manufactured by Agilent Corporation (Santa Clara, CA, USA). Capillary electrophoresis methods are also suitable for measuring other RNA qualities such as polyA tail length and uniformity (Di Grandi 2023, Tu 2024). RNA integrity can also be addressed using reverse transcription-qPCR (Poveda 2019, Di Grandi 2023). Double-stranded RNA present in RNA synthesized in in vitro transcription reactions is quantitated using dot blots or ELISA assays based on monoclonal antibodies that specifically bind double-stranded RNA (Aramburu 1991, Karikó 2011, Baiersdörfer 2019), such as the J2 IgG2a monoclonal antibody and the and the IgG2a K1 and IgM K2 monoclonal antibodies (Schonborn 1991) and the 9D5 monoclonal antibody (Son 2015). Double-stranded RNA levels can also be determined using reverse transcription-qPCR (Poveda 2019, Di Grandi 2023). Capping efficiency and cap incorporation efficiency can be measured with a variety of methods including gel electrophoresis, fluorescence spectroscopy (when using fluorescently labeled cap analogs), nanopore sequencing and liquid chromatography-mass spectrometry (Tu 2024). RNA polymerase and RNA fidelity are addressed by a variety of sequencing methods, including RNA sequencing following reverse transcription and nanopore sequencing (Gholamalipour 2018, Poveda 2019, Gunter 2023). RNA quality is also measured by in vitro translation followed by enzymatic assays (for RNAs encoding enzymes whose activity can be determined in vitro) and cell-based assays (Poveda 2019).

The present disclosure provides compositions and methods for screening systems that employ nucleic acid templates comprising dual promoter elements for nucleic acid polymerases. These screening systems can be used both in vitro (in cell-free systems) or in vivo (within living cells) to facilitate the isolation of genes encoding improved enzymes and the discovery of nucleic acid sequences that enhance gene expression.

A simple aspect of the disclosure comprises a nucleic acid template that encodes a nucleic acid polymerase, for example an RNA polymerase (RNApol). The open reading frame (ORF) or protein coding sequence that encodes the RNA polymerase is referred to as the RNA polymerase ORF. The RNA polymerase encoded by the RNA polymerase ORF is referred to as the target RNA polymerase.

Upstream (5′) of the ORF encoding the RNA polymerase lie two promoters specific for RNA polymerases. One of these promoters (the initiating RNA polymerase promoter) is specific for an initiating RNA polymerase which is combined with the template nucleic acid at the start of the process or reaction. The other promoter (the target RNA polymerase promoter) is specific for the RNA polymerase encoded by the RNA polymerase ORF present in the nucleic acid template. The initiating RNA polymerase added to the reaction or process recognizes the initiating RNA polymerase promoter and transcribes the nucleic acid template to synthesize an RNA transcript (the initiating transcript) which encodes the target RNA polymerase. The initiating transcript is translated into the encoded protein, the target RNA polymerase, which in turn recognizes the target RNA polymerase promoter and transcribes the nucleic acid template to synthesize a second RNA transcript (the target transcript), distinct from the initiating transcript, which also encodes the target RNA polymerase.

After the reaction is allowed to go to completion, RNA is isolated and reverse transcribed into cDNA which can then be amplified, either in whole or in part, by PCR. Sequence differences between the initiating transcript and the target transcript allow specific PCR amplification of the target transcript, or specific sequences contained within it. These sequences can be cloned or characterized in any manner, or they can be incorporated into a second nucleic acid template that is used for another round of screening. This process enriches for sequence encoded by the nucleic acid template that has specific properties of interest to biotechnology.

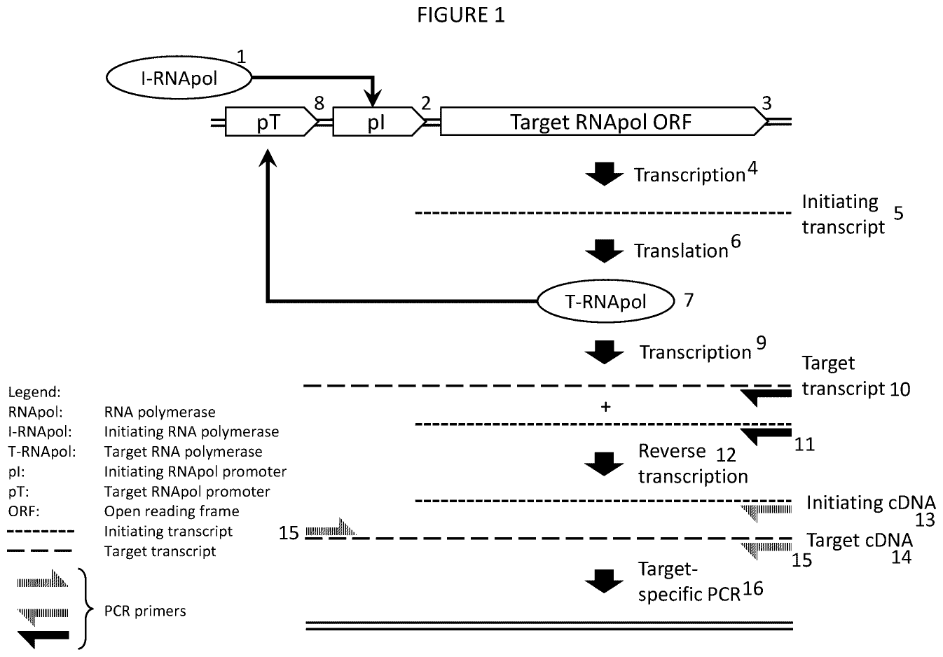

This simple aspect of the disclosure is diagrammed schematically in FIG. 1.

In the screening system described above, the nucleic acid template created from RNA molecules isolated from an initial round of screening, can be constructed in a manner to replicate or minic the structure of the nucleic acid template used in the initial round of screening. Alternatively, the structure can be changed between screening rounds. This may allow flexible screening for multiple qualities of the RNA polymerase or multiple qualities of the template molecule, in successive screening rounds.

The disclosure can be combined with an emulsion-based reaction system (such as an oil in water emulsion consisting of 10⁹ or more aqueous droplets of 2 micrometers average diameter, more detailed examples described in the references given in the Background section above), in which the reaction is separated or compartmentalized into a population of micro-droplets that serve to separate transcription and translation of different variant nucleic acid template molecules. Emulsion-based systems can be a powerful way of running the equivalent of millions of separate reactions in a small reaction volume present in a single tube.

The disclosure can be used to discover or evolve a variety of sequences of interest to biotechnology. For example, the screening system can be used to enrich for target RNA polymerases with higher transcriptional activity from a specific promoter. The nucleic acid template can be constructed in a manner that it encodes a variety of mutated or otherwise altered RNA polymerase ORFs (variant RNA polymerases). The ORFs encoding more efficient target RNA polymerases will generate a higher number of target transcripts which will be amplified at the end of the screening rounds, increasing the prevalence of RNAs encoding efficient RNA polymerases in the overall population of variant transcripts. Multiple rounds of screening will result in enrichment of the most efficient RNA polymerases.

Qualities of nucleic acid polymerases or RNA polymerases that are of interest to biotechnology and that can be improved in the screening system described in this invention include, but are not limited to: transcription initiation from a specific promoter; transcriptional activity from a specific promoter or template nucleic acid; the ability to synthesize a specific number of RNA transcripts from a single nucleic acid template molecule within a specific time interval; yield of RNA synthesized in a reaction in which the RNA polymerase is used to transcribe a specific nucleic acid template; ability to incorporate modified nucleotides or nucleotides not typically incorporated by RNA polymerases such as nucleotides with modified sugars, bases, phosphodiester linkages, or deoxyribonucleotides, into a nucleic acid strand; ability to synthesize non-RNA nucleic acids such as DNA; RNA integrity (that is, the percentage of full-length RNA transcribed from a nucleic acid template relative to total RNA) of RNA synthesized in a reaction in which the RNA polymerase is used to transcribe a specific nucleic acid template; altered amounts of or the absence of undesirable side products, including but not limited to RNA transcripts shorter than the full-length transcript, antisense RNA molecules, or double-stranded RNA synthesized in a reaction in which the RNA polymerase is used to transcribe a specific nucleic acid template; incorporation of 1 or 2 phosphate groups at the transcriptional 5′ end; efficiency of incorporation of a 7-methyl-guanosine cap at the 5′ end of an RNA molecule, or of a different nucleotide cap, a di-nucleotide cap, tri-nucleotide cap, or a longer cap used to initiate synthesis of RNA molecules synthesized in a reaction in which the RNA polymerase is used to transcribe a specific nucleic acid template; processivity of an RNA polymerase; ability of an RNA polymerase to synthesize long RNAs, for example RNAs in excess of 5 kb; RNA polymerase activity at a certain temperature; heat tolerance of an RNA polymerase; tolerance of an RNA polymerase to salts or other potentially inhibitory compounds present in an in vitro transcription reaction; or any other quality of the RNA polymerase that affects or alters its activity in the synthesis of RNA molecules or other nucleic acid molecules.

Another aspect of the disclosure can be used to select for RNA polymerase variants that more efficiently use a mutated target promoter. In this case, the template nucleic acid comprises one or more mutated target promoters and variant RNA polymerases. The screening system can be used to enrich or select for genes encoding variant RNA polymerases with higher efficiency of recognizing the mutated target promoter.

Another aspect of the disclosure can be used to select for RNA polymerase variants that more efficiently use an alternative or mutated transcriptional start site. In this case, the template nucleic acid comprises one or more mutated transcriptional start sites and variant RNA polymerases. The mutated transcriptional start site can correspond to a transcriptional start site at which the target RNA polymerase is not able to initiate, or at which the target RNA polymerase initiates inefficiently, or at which the target RNA polymerase initiates efficiently. The screening system can be used to enrich or select for genes encoding variant RNA polymerases with higher efficiency of initiating transcription at the mutated transcriptional start site.

Another aspect of the disclosure can be used to select for promoter sequences recognized by the initiating RNA polymerase. In this embodiment of the invention, the template molecules used in the screen will differ from each other in the sequence of the initiating RNA polymerase promoter. For example, a diverse collection of sequences can be incorporated into the position of the initiating RNA polymerase promoter. This could be a partially or completely degenerate sequence of any number of nucleotides, for example 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000 or longer sequence stretch in which one or more nucleotides are varied. The resulting collection of template molecules, when used in the screen described above, will select for template molecules containing promoter sequences recognized by the initiating RNA polymerase.

Another aspect of the disclosure can be used to select for untranslated sequences that more efficiently direct protein expression from an RNA transcript. In this case, the template nucleic acid comprises one or more degenerate, randomized or otherwise diversified untranslated sequences. The screening system can be used to enrich or select for untranslated sequences that efficiently direct protein expression. High-efficiency untranslated sequences cause higher expression of the target RNA polymerase which raises the transcript level from the target promoter. This raises the abundance of the high-efficiency untranslated sequence(s) in the overall population of variant sequences.

Untranslated sequences that can be discovered using this method include 5′ untranslated sequences (5′ UTRs, or the sequences between the transcription initiation site and the start codon of the RNA polymerase ORF), Kozak sequences or other ribosome binding sites that recruit ribosomes to the start codon of the RNA polymerase ORF, 3′ untranslated sequences (3′ UTRs, or the sequences between the stop codon of the RNA polymerase ORF and the end of the nucleic acid template), poly-A sequences encoded in the nucleic acid template, or any other sequences present in the nucleic acid template that do not encode a peptide, polypeptide, or protein.

The promoters encoded by the nucleic acid template used for the screening systems described in the present disclosure can be any promoter recognized by any RNA polymerase from any organism. In one aspect of the disclosure, the target RNA polymerase promoter is a specific promoter sequence recognized by a specific single-subunit RNA polymerase. In another aspect of the disclosure, the initiating RNA polymerase promoter is a specific promoter sequence recognized by a specific single-subunit RNA polymerase. In the examples given in the present disclosure, the initiating RNA polymerase promoter and the target RNA polymerase promoters are distinct and recognized by distinct RNA polymerases.

The initiating RNA polymerase promoter and the target RNA polymerase promoter can be located at any position within the nucleic acid template used in the screening system described herein. Both promoters need to be located upstream of the target RNA polymerase ORF, but otherwise can have any position in relation to the untranslated sequences or other ORFs present in the nucleic acid template. In one aspect of the disclosure, the target RNA polymerase promoter is located upstream of the initiating RNA polymerase promoter which facilitates isolation of cDNAs derived from transcripts originating from the target RNA polymerase promoter, for example by specifically PCR amplifying the cDNAs with primers homologous to sequences located directly downstream of the target RNA polymerase promoter (for example, see FIG. 1). The target RNA polymerase promoter and initiating RNA polymerase promoter can be separated by any sequences such as untranslated sequences or ORFs.

The target RNA polymerase promoter and initiating RNA polymerase promoter can be any distance apart within the nucleic acid template used in the screening system described herein. For example, they can be separated by 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 nucleotides or more, or any number in between.

Different aspects of the disclosure use different numbers of ORFs in the nucleic acid template used to screen for desirable sequences. The nucleic acid template needs to encode at least one ORF (the target RNA polymerase ORF) but can encode any number of additional ORFs, including a total of 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000 ORFs or more, or any number in between. The ORFs present in addition to the target RNA polymerase ORF can encode any protein or enzyme of any type, including natural, synthetic, or chimeric.

In cases where the nucleic acid template used in the screening system described herein encodes more than one ORF (meaning one ORF encoding a target RNA polymerase and the other ORF(s) encoding other proteins), the target RNA polymerase ORF can be positioned 5′ of the other ORFs or 3′ to it or in between other ORFs. When positioned in between, it can be present at any position. For example, when a nucleic acid template encodes a total of 5 ORFs, with the ORFs numbered 1 through 5 starting at the 5′ position, the target RNA polymerase ORF can be located at position 1, 2, 3, 4 or 5.

When multiple ORFs are present in the nucleic acid template used in the screening system described herein, the different ORFs can be overlapping or separated by non-coding (untranslated) sequences.

When multiple ORFs are present in the nucleic acid template used in the screening system described herein, the different ORFs can be separated by any amount of non-coding or untranslated sequence, including 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 nucleotides or more, or any number in between. The non-coding or untranslated sequences can have any sequence, natural or synthetic, complex or repetitive or combinations thereof. The non-coding or untranslated sequences separating different ORFs can be the same or different, unrelated or related to each other.

The target RNA polymerase ORF can also be present in multiple copies within the ORFs present on a nucleic acid template. For example, the target RNA polymerase ORF can be present in 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000 copies or more, or any number in between. The different copies of the target RNA polymerase ORF can have the same sequence or can differ in their sequences. The different copies of target RNA polymerase ORFs can be present in a tandem array or in arrays of inverted repeats or in different positions within the nucleic acid template, or combinations thereof.

### EXAMPLES

### Example 1: Emulsion-Based Screening for Improved RNA Polymerases

### RNA Polymerases, Enzyme Expression, and Enzyme Purification

An open reading frame encoding the initiating RNA polymerase (RNApol) is cloned into a bacterial expression plasmid with an MB1 plasmid replicon conferring a high copy number in E. coli. The insertion site for the RNA polymerase gene on the plasmid is flanked by an arabinose inducible promoter and a Lambda t1 terminator, allowing for arabinose-inducible expression of the polymerase. The expression construct is sequence verified after cloning. Depending on the initiating promoter of the screening template (described later), the initiating RNApol may be RNApol031 (SEQ ID NO:69), RNApol136 (SEQ ID NO:70), or RNApol137 (SEQ ID NO:71). The full sequences of the expression constructs for the initiating RNA polymerases covered in this disclosure are given in SEQ ID NO:42 (RNApol137), SEQ ID NO:43 (RNApol136) and SEQ ID NO:44 (RNApol031). Each of the initiating RNApols contains an N-terminal His6 tag to aid in RNApol purification.

To produce the purified initiating RNA polymerase RNApol031 (SEQ ID NO:69), the RNApol031 expression plasmid given in SEQ ID NO:44 is transformed into the E. coli strain BL21 and a single colony picked for cultivation and protein expression. The bacterial cells are grown in LB medium at 30° C. to log phase culture and induced by addition of L-arabinose. After 18 hours of incubation at 16° C., the cultures are harvested by centrifugation and the collected E. coli cells are lysed. RNA polymerase is purified with metal affinity chromatography according to manufacturer's instructions. The RNA polymerase is eluted with imidazole solution, concentrated with AMICON® Ultra-centrifugal filter sold by Millipore (Darmstadt, Germany) and transferred into a storage buffer composed of 50 mM Tris pH8.0, 75 mM NaCl, 0.5 mM EDTA, and 50% glycerol.

To produce the purified initiating RNA polymerase RNApol136 (SEQ ID NO:70), or RNApol137 (SEQ ID NO:71), the corresponding expression plasmid is transformed into the E. coli strain BL21 and a single colony picked for cultivation and protein expression. The bacterial cells are grown in LB medium at 37° C. to log phase culture and induced by addition of L-arabinose. After 18 hours of incubation at 15° C., the cultures are harvested by centrifugation and the collected E. coli cells are lysed. RNA polymerase is purified with metal affinity chromatography according to manufacturer's instructions. The RNA polymerase is eluted with imidazole solution, concentrated with AMICON® Ultra-centrifugal filter sold by Millipore (Darmstadt, Germany) and changed into a storage buffer composed of 50 mM KPO4, pH7.3, 100 mM NaCl, 1.43 mM Beta mercaptoethanol, 0.05% Triton-X100, and 50% glycerol.

### Design and Construction of DNA Templates for In Vitro Screening:

DNA templates for the in vitro screen contain the following significant features in the following order: A promoter, transcription start site, and 5′ UTR for the target RNA polymerase, a promoter, transcription start site, and 5′ UTR for the initiating RNA polymerase, ribosome binding site, spacer, His tag, the gene encoding the RNA polymerase of interest, and a terminator (see FIG. 1). When these templates are used in an in vitro expression reaction, the initiating RNA polymerase added at the reaction onset will transcribe RNA encoding the RNA polymerase of interest, reaction components will express the RNA polymerase of interest, and that RNA polymerase of interest will act upon its own promoter, transcribing an RNA molecule that is longer than and distinct from those transcripts produced by the initiating RNA polymerase. During subsequent PCR steps, this distinction allows for the selective amplification of molecules originating from transcripts produced by the RNA polymerase of interest and the exclusion of initiating-RNApol-derived material.

The sequences of the DNA templates used in the in vitro screen described in this example are given in SEQ ID NO:1, SEQ ID NO:2, SEQ ID NO:3, SEQ ID NO:4, SEQ ID NO:5, SEQ ID NO:6, SEQ ID NO: 23, SEQ ID NO: 24, SEQ ID NO: 25, SEQ ID NO: 39, SEQ ID NO: 40, and SEQ ID NO: 41. The six screening templates given in SEQ ID NO:1, SEQ ID NO:2, SEQ ID NO: 3, SEQ ID NO:4, SEQ ID NO:5 and SEQ ID NO:6 were generated by homology-dependent assembly reaction (Gibson 2009, Gibson 2010) from various source fragments previously cloned into plasmid vectors or ordered from commercial gene synthesis suppliers. The fragments for SEQ ID NO:1 were amplified using the following PCR primers prior to assembly. Fragment 1, containing all sequences upstream of the RNApol137 (SEQ ID NO:71) ORF was amplified with PCR primers given in SEQ ID NO:11 and SEQ ID NO:58. Fragment 2, which encodes the open reading frame encoding the target RNA polymerase RNApol137 (SEQ ID NO: 71), was PCR amplified over 19 cycles from the a source plasmid containing the cloned open reading frame encoding the target RNA polymerase RNApol137 (SEQ ID NO:71) using the GENEMORPH® II Random Mutagenesis Kit (Agilent, Santa Clara, CA) with primers given in SEQ ID NO:7 and SEQ ID NO:8. Fragment 3, which contains the terminator, was PCR amplified from a source plasmid containing this terminator sequence using primers given in SEQ ID NO:9 and SEQ ID NO:10. Amplicons from plasmids were subjected to DpnI digestion, and all amplicons were gel purified using the NUCLEOSPIN® Gel and PCR Clean-up Kit (Macherey-Nagel, Düren, Germany). All three fragments were assembled in an assembly reaction (Gibson 2009, Gibson 2010), after which assembled products were PCR amplified using primers given in SEQ ID NO:10 and SEQ ID NO:11. Final products were also purified via gel electrophoresis as described above.

The screening templates given in SEQ ID NO:2, SEQ ID NO:3, SEQ ID NO:4, SEQ ID NO:5 and SEQ ID NO:6 are created in an identical manner to the screening template given in SEQ ID NO: 1 except that certain template-specific primers are used. Primers for fragment #3 are identical among all 6 templates. For the screening template given in SEQ ID NO:2, the primer sequences given in SEQ ID NO:46 and SEQ ID NO:52 are used for fragment #1, the primer sequences given in SEQ ID NO:8 and SEQ ID NO:53 are used for fragment #2, and the primer sequences given in SEQ ID NO:10 and SEQ ID NO:46 are used in post-assembly PCR of the entire template. For the screening template given in SEQ ID NO:3, the primer sequences given in SEQ ID NO:45 and SEQ ID NO:50 are used for fragment #1, the primer sequences given in SEQ ID NO: 8 and SEQ ID NO:51 are used for fragment #2, and the primer sequences given in SEQ ID NO: 10 and SEQ ID NO:45 are used for post-assembly PCR of the entire template. For the screening template given in SEQ ID NO:4, the primer sequences given in SEQ ID NO:47 and SEQ ID NO:54 are used for fragment #1, the primer sequences given in SEQ ID NO:8 and SEQ ID NO: 55 are used for fragment #2, and the primer sequences given in SEQ ID NO:10 and SEQ ID NO: 47 are used for post-assembly PCR of the entire template. For the screening template given in SEQ ID NO:5, the primer sequences given in SEQ ID NO:49 and SEQ ID NO:59 are used for fragment #1, the primer sequences given in SEQ ID NO:8 and SEQ ID NO:60 are used for fragment #2, and the primer sequences given in SEQ ID NO:10 and SEQ ID NO:49 are used for post-assembly PCR of the entire template. For the screening template given in SEQ ID NO:6, the primer sequences given in SEQ ID NO:48 and SEQ ID NO:56 are used for fragment #1, the primer sequences given in SEQ ID NO:8 and SEQ ID NO:57 are used for fragment #2, and the primer sequences given in SEQ ID NO:10 and SEQ ID NO:48 are used for post-assembly PCR of the entire template.

The screening templates given in SEQ ID NO:39, SEQ ID NO:40 and SEQ ID NO: 41 are created via two-step PCR. Amplicons from plasmids are subjected to DpnI digestion, and all amplicons are purified after each PCR step via gel electrophoresis as described above. For the screening template given in SEQ ID NO:39, the RNA polymerase coding sequence is amplified and mutagenized over 20 cycles with the primer sequences given in SEQ ID NO:30 and SEQ ID NO:32 using the GENEMORPH® II Random Mutagenesis Kit (Agilent, Santa Clara, CA). The full template given in SEQ ID NO:39 is generated from the resulting fragments via standard PCR with the primer sequences given in SEQ ID NO:26 and SEQ ID NO:29. An identical process is used to create the screening templates given in SEQ ID NO:40 and SEQ ID NO:41, except that template-specific primers are substituted as follows: For SEQ ID NO:40, the primer sequence given in SEQ ID NO:31 is substituted for the primer sequence given in SEQ ID NO:32 and the primer sequence given in SEQ ID NO:27 is substituted for the primer sequence given in SEQ ID NO:26. For SEQ ID NO:41, the primer sequence given in SEQ ID NO:28 is substituted for the primer sequence given in SEQ ID NO:26.

The sequence of the screening template given in SEQ ID NO:1 contains the following sequences elements with the nucleotide positions indicated: 21-34: RNApol137 promoter; 35-36: RNApol137 transcription initiation site; 71-84: RNApol031 promoter; 85-86: RNApol031 transcription initiation site; 105-112: E. coli ribosome binding site; 119-2776: RNApol137 ORF; 2801-2891: bacteriophage lambda t1 terminator.

Annotations of sequences present in various other sequences described in this invention are as follows:
SEQ ID NO:2, 21-34: RNApol031 promoter; 35-36: RNApol031 transcription initiation site; 75-88: RNApol137 promoter; 89-90: RNApol137 transcription initiation site; 105-111 E. coli ribosome binding site; 119-2554: RNApol031 ORF; 2579-2668: bacteriophage lambda t1 terminator.
SEQ ID NO:3, 21-32: RNApol002 promoter; 33-34: RNApol002 transcription initiation site; 68-81: RNApol031 promoter; 82-83: RNApol031 transcription initiation site; 102-108 E. coli ribosome binding site; 116-2572: RNApol002 ORF; 2597-2686: bacteriophage lambda t1 terminator.
SEQ ID NO:4, 21-38: RNApol126 promoter; 39-40: RNApol126 transcription initiation site; 88-101: RNApol031 promoter; 102-103: RNApol031 transcription initiation site; 122-128: E. coli ribosome binding site; 136-2787: RNApol126 ORF; 2812-2901: bacteriophage lambda t1 terminator.
SEQ ID NO:5, 21-37: RNApol161 promoter; 38-39: RNApol161 transcription initiation site; 80-93: RNApol031 promoter; 94-95: RNApol031 transcription initiation site; 114-120: E. coli ribosome binding site; 128-2803: RNApol161 ORF; 2828-2917: bacteriophage lambda t1 terminator.
SEQ ID NO:6, 21-37: RNApol136 promoter; 38-39: RNApol136 transcription initiation site; 80-93: RNApol031 promoter; 94-94: RNApol031 transcription initiation site; 114-120: E. coli ribosome binding site; 128-2779: RNApol136 ORF; 2804-2893: bacteriophage lambda t1 terminator.
SEQ ID NO:18, 201-290: araC terminator; 291-927: araC ORF; 1401-1495: ara promoter; 1497-1504: E. coli ribosome binding site; 1511-2676: T7 RNA polymerase ORF; 4211-4300: bacteriophage lambda t1 terminator; 4649-5506: Ampicillin resistance gene; 5780-6268: pMB1 origin of replication.
SEQ ID NO:19, 95-111: T7 RNA polymerase promoter; 132-203: Human myoglobin 5′ untranslated region; 204-3275: E. coli lacZ gene; 3288-4937: firefly luciferase gene; 4938-5079: human beta globin 3′ untranslated region; 5070-5198: poly-A tail; 5577-6437: ampicillin resistance gene; 6608-7196: pMB1 origin of replication.
SEQ ID NO:23, 21-43: RNApol157 promoter; 44-46: RNApol157 transcription initiation site; 77-93: RNApol136 promoter; 94-96: RNApol136 transcription initiation site; 116-121: E. coli ribosome binding site; 130-2805: RNApol157 ORF; 2806-2887: modified bacteriophage lambda t1 terminator.
SEQ ID NO:24, 21-43: RNApol157 promoter; 44-46: RNApol157 transcription initiation site; 77-93: RNApol136 promoter; 94-96: RNApol136 transcription initiation site; 116-121: E. coli ribosome binding site; 130-2805: RNApol157 ORF; 2806-2887: modified bacteriophage lambda t1 terminator.
SEQ ID NO:25, 21-44: RNApol126 promoter; 45-47: RNApol126 transcription initiation site; 78-94: RNApol136 promoter; 95-97: RNApol136 transcription initiation site; 117-122: E. coli ribosome binding site; 131-2783: RNApol126 ORF; 2783-2864: modified bacteriophage lambda t1 terminator.
SEQ ID NO:39, 21-43: RNApol157 promoter; 44-46: RNApol157 transcription initiation site; 77-93: RNApol136 promoter; 94-96: RNApol136 transcription initiation site; 116-121: E. coli ribosome binding site; 130-2805: RNApol157 ORF.
SEQ ID NO:40, 21-44: RNApol126 promoter; 45-47: RNApol126 transcription initiation site; 78-94: RNApol136 promoter; 95-97: RNApol136 transcription initiation site; 117-122: E. coli ribosome binding site; 131-2783: RNApol126 ORF.
SEQ ID NO:41, 21-43: RNApol157 promoter; 44-46: RNApol157 transcription initiation site; 77-93: RNApol136 promoter; 94-96: RNApol136 transcription initiation site; 116-121: E. coli ribosome binding site; 130-2805: RNApol157 ORF.
SEQ ID NO:42, 201-290: AraC terminator; 291-1217: araC gene; 1401-1510: Ara promoter; 1511-4168: RNApol137 ORF; 4193-4282: bacteriophage lambda t1 terminator; 4631-5488: ampicillin resistance gene; 5662:6250: pmb1 origin of replication.
SEQ ID NO:43, 201-290: AraC terminator; 291-1217: araC gene; 1401-1510: Ara promoter; 1511-4162: RNApol136 ORF; 4187-4276: bacteriophage lambda t1 terminator; 4625-5482: ampicillin resistance gene; 5656-6244: pmb1 origin of replication.
SEQ ID NO:44, 201-290: AraC terminator; 291-1217: araC gene; 1401-1510: Ara promoter; 1511-3946: RNApol031 ORF; 3971-4060: bacteriophage lambda t1 terminator; 4409-5266: ampicillin resistance gene; 5440-6028: pmb1 origin of replication.

The design of the secondary screen (described below) includes an initiating construct containing a promoter, transcription start site, and 5′ UTR for the initiating RNA polymerase followed by a ribosome binding site, linker, a gene encoding the His-tagged RNA polymerase of interest, and a terminator. A second necessary component of this screen is a reporter construct containing a promoter, transcription start site, and 5′ UTR for the RNA polymerase of interest, followed by a ribosome binding site, linker, reporter genes (such as LacZ and luciferase), and a terminator. During the secondary screen, in-vitro-expressed RNA polymerase of interest will transcribe RNA encoding for lacZ and luciferase in an activity-dependent manner, after which qPCR can provide an estimate of that activity.

### Enrichment of Improved RNA Polymerases Via Iterative Rounds of In Vitro Screening:

An oil-surfactant mixture is prepared by adding Span-80 and Tween-80 to mineral oil to final concentrations of 4.5% and 0.5%, respectively (Miller, 2006). Aqueous reaction mixture is then added as 5 aliquots of 10 μL over a period of 2 minutes to 950 μL of the oil-surfactant mixture while stirring at 1,500 r.p.m. on ice to create an emulsion. The aqueous reaction mixture consists of Solution A and Solution B from a custom PUREXPRESS® In vitro Protein Synthesis Kit that lacks T7 RNA polymerase (New England Biolabs, Ipswich, MA), 1 μL RNase inhibitor from human placenta (New England Biolabs, Ipswich, MA), 50 ng of initiating RNA polymerase, 60 ng of a DNA library, and nuclease-free water to 50 μL. After addition of the final aliquot of the aqueous reaction mixture, stirring is continued under the same conditions for an additional minute. Samples are stirred on ice for an additional 3 minutes at 8,000 r.p.m. on an ULTRA TURRAX® T8 homogenizer (Ika, Staufen, Germany). Emulsified samples are incubated for 3 h at 30° C. while shaking.

To recover RNA transcripts generated within the emulsions, 50 μL of nuclease-free water and 500 μL TRIzol Reagent (Invitrogen, Waltham, MA), are mixed with samples and incubated for 5 minutes at room temperature. Samples are frequently frozen at −80° C. at this point. After thawing if necessary, 800 μL of ethanol is mixed with samples and samples are purified on silica columns using the DIRECT-ZOL™ RNA Miniprep Plus (Zymo, Irvine, CA) kit, according to the manufacturer's instructions. Samples are eluted with 90 μL of nuclease-free water. Samples are heated at 75° C. for 5 minutes, after which 10 μL of 10× DNase I Reaction Buffer and 2 μL of DNase (New England Biolabs, Ipswich, MA) is added to 88 μL of each sample. Samples are incubated at 37° C. for 30 minutes. The RNA CLEAN AND CONCENTRATOR™-5 kit (Zymo, Irvine, CA) used to further purify each sample, and RNA is eluted in 15 μL of nuclease-free water. A 2 μL aliquot of each sample is combined with 1 μL of 10 μM primer given in SEQ ID NO:22 and incubated at 70° C. for 5 minutes. Reverse transcription is performed by use of the AFFINITYSCRIPT® cDNA Synthesis Kit (Agilent, Santa Clara, CA) according to the manufacturer's instructions. Control reactions lacking reverse transcriptase are performed for each sample. Brilliant III Ultra-Fast SYBR® Green QPCR Master Mix (Agilent, Santa Clara, CA) is used according to the manufacturer's instructions to quantify levels of total and target transcripts on an ARIAMX® Real-Time PCR System. For qPCR reactions quantifying cDNA derived from the screening template given in SEQ ID NO:1, the primer sequences given in SEQ ID NO:12 and SEQ ID NO:13 are used to quantify target transcripts; the primer sequences given in SEQ ID NO: 14 and SEQ ID NO:15 are used to quantify total transcripts. For the screening templates given in SEQ ID NO:23, SEQ ID NO:24, SEQ ID NO:25, SEQ ID NO:39, SEQ ID NO:40 and SEQ ID NO: 41, the primer sequence given in SEQ ID NO:33 is used in the place of the primer sequence given in SEQ ID NO: 12. For the screening templates given in SEQ ID NO:4, SEQ ID NO:25 and SEQ ID NO:40, the primer sequences given in SEQ ID NO:34 and SEQ ID NO:35 are used to quantify total transcripts. For the screening templates given in SEQ ID NO:23, SEQ ID NO:24, SEQ ID NO:39 and SEQ ID NO:41, the primer sequences given in SEQ ID NO:34 and SEQ ID NO: 36 are used to quantify total transcripts.

The cDNA generated from emulsions using the screening template given in SEQ ID NO: 1 is selectively PCR amplified using the primer sequences given in SEQ ID NO:16 and SEQ ID NO:17, which restore the template components upstream of the transcription start site (TSS) and allow the resulting product to be used in a subsequent screening cycle. The primer sequence given in SEQ ID NO: 16 is replaced with the primer sequence given in SEQ ID NO:61 for template regeneration for the screening template given in SEQ ID NO:2, with the primer sequence given in SEQ ID NO:62 for template regeneration for the screening template given in SEQ ID NO:3, with the primer sequence given in SEQ ID NO:63 for template regeneration for the screening template given in SEQ ID NO:4, with the primer sequence given in SEQ ID NO:64 for template regeneration for the screening template given in SEQ ID NO:5, and with the primer sequence given in SEQ ID NO:65 for template regeneration for the screening template given in SEQ ID NO:6. The primer sequences given in SEQ ID NO:9 and SEQ ID NO:10 are used in a PCR reaction using the plasmid given in SEQ ID NO:18 as a template to create a DNA fragment containing a terminator, and this amplicon is combined in an assembly reaction (Gibson 2009, Gibson 2010) with product from the previous step to yield full length template. After assembly, full-length template is amplified in a PCR reaction using the primer sequences given in SEQ ID NO: 10 and SEQ ID NO:38. PCR products are purified via gel electrophoresis using the NUCLEOSPIN® Gel and PCR Clean-Up kit (Macherey-Nagel, Düren, Germany).

The screening templates given in SEQ ID NO:39, SEQ ID NO:40 and SEQ ID NO: 41 are regenerated after the first round of cDNA synthesis by PCR amplification from cDNA using primer pairs identical to those used in the earlier template generation step immediately following mutagenic PCR. In subsequent enrichment rounds, the primer sequence given in SEQ ID NO: 37 is used instead of the primer sequence given in SEQ ID NO:29, which converts the template sequence given in SEQ ID NO:39 into the template sequence given in SEQ ID NO:24, converts the template sequence given in SEQ ID NO:40 into the template sequence given in SEQ ID NO: 25 and converts the template sequence given in SEQ ID NO:41 into the template sequence given in SEQ ID NO:23. Low target yields can occur in some enrichment rounds and can be overcome with an additional PCR step using the primers given in SEQ ID NO:10 and SEQ ID NO: 38. The process beginning with the creation of an emulsion and culminating in the purification of these PCR products constitutes one cycle of enrichment, after which samples may be subjected to one or more additional rounds of enrichment or taken forward for isolation of hits from the enriched libraries.

The frequency of specific mutations over multiple rounds of enrichment for improved polymerases was tracked via next-generation sequencing. LGC Biosearch Technologies (Teddington, United Kingdom) provided incoming amplicon DNA quality control, library preparation, library quality control, and sequencing on 1 NextSeq MO Cartridge with 2×150 bp reagents. LGC Biosearch Technologies also provided bioinformatic analysis of sequencing data.

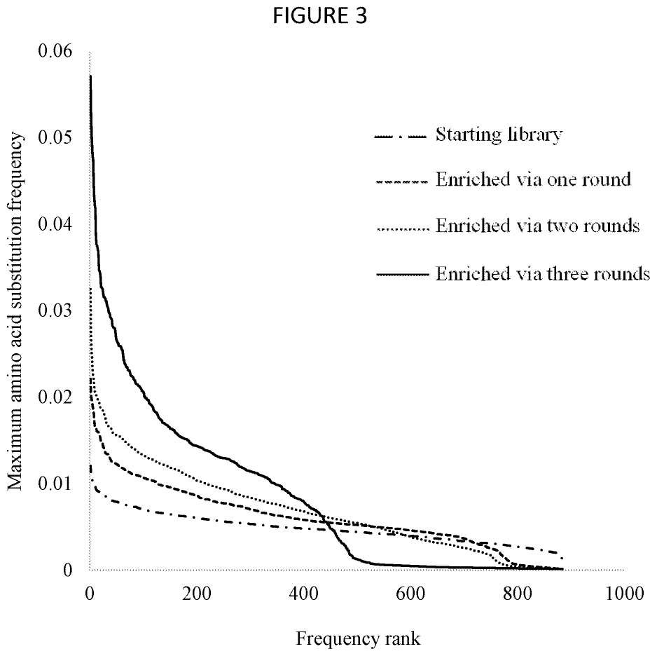

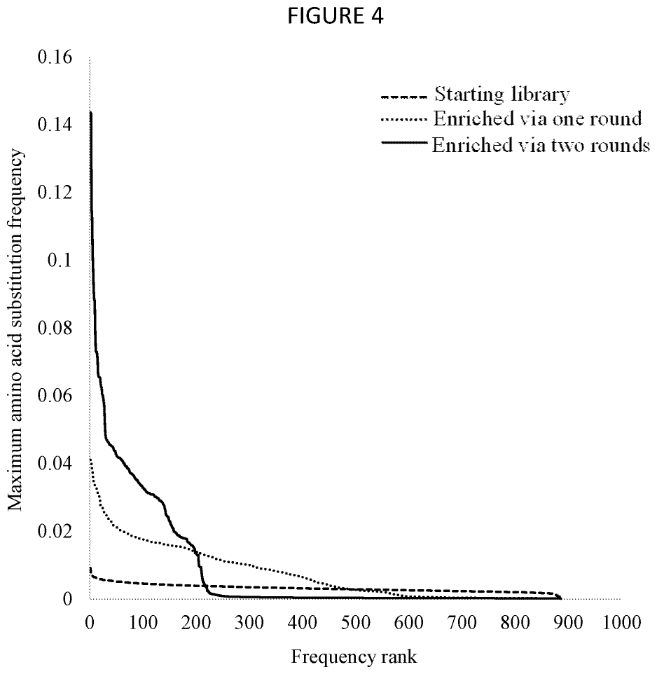

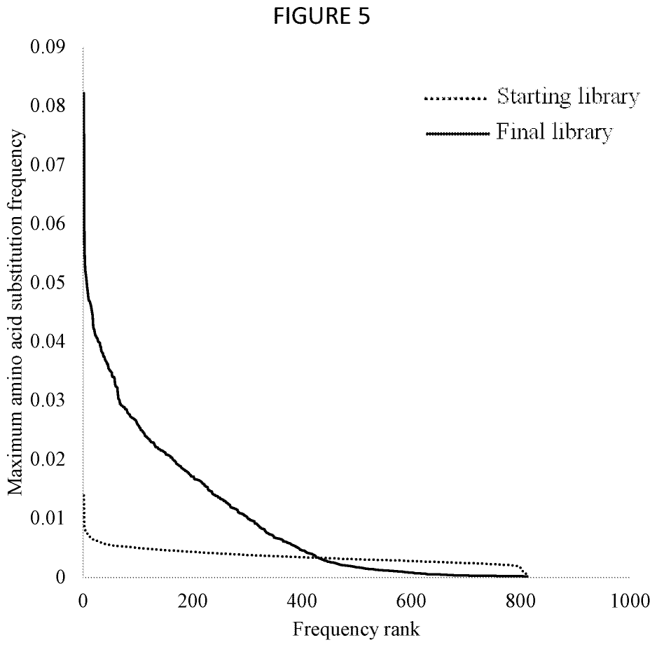

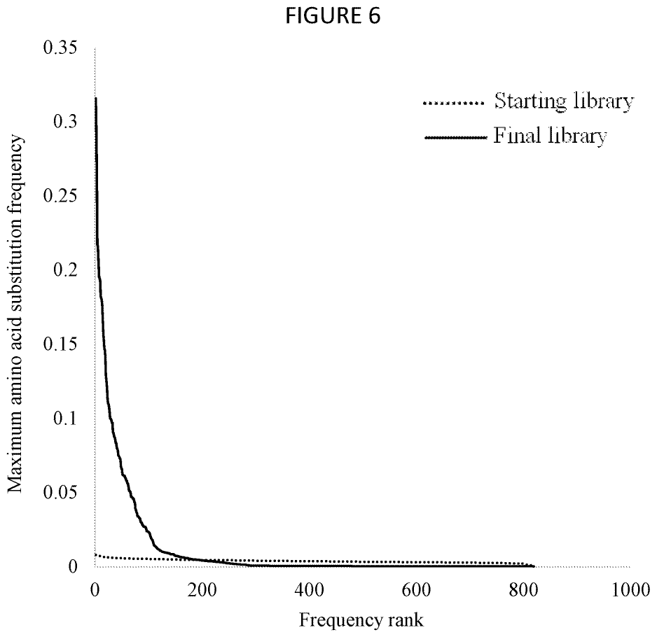

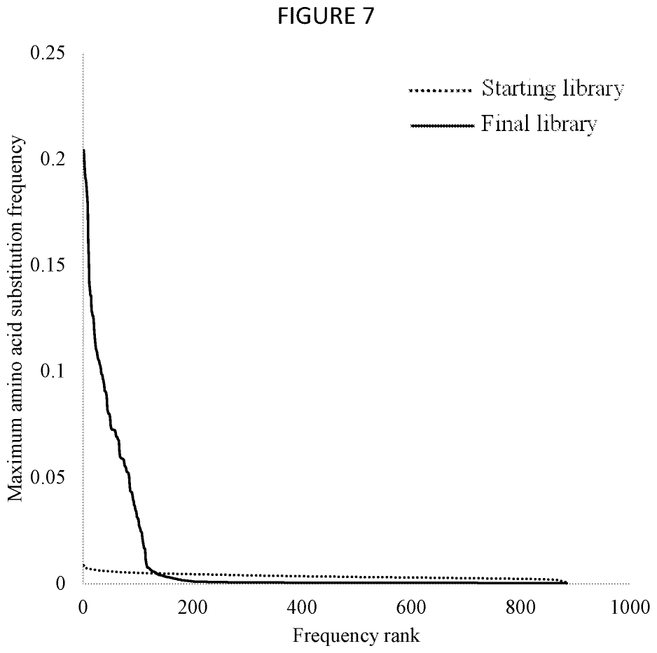

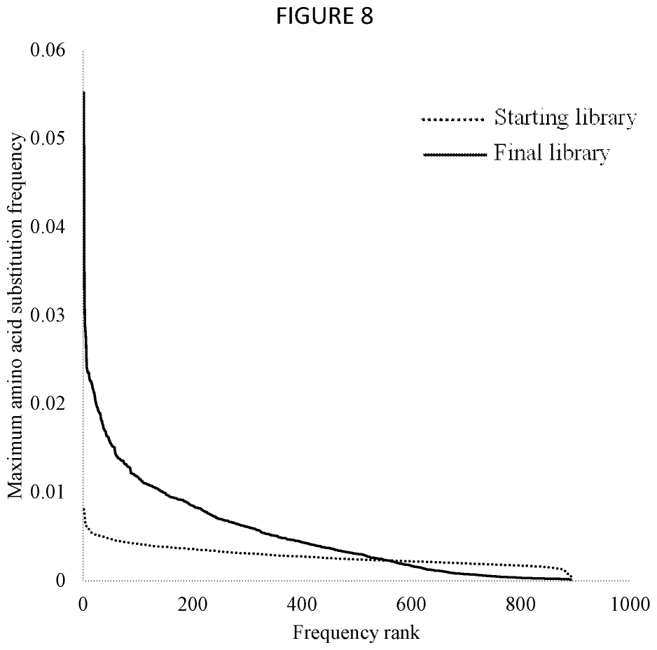

Next-generation sequencing results show that amino acid substitutions at certain positions increase significantly over multiple iterative rounds of screening (FIGS. 3-8). Several of these single-amino-acid substitutions are estimated to be present in over 20% of library members after multiple rounds of screening, despite a substitution frequency well below 1% for nearly all residues in the starting library. In contrast, the majority of positions show no increase in substitution frequency or show a decrease in frequency of the most prevalent substitution compared to the starting library. These results align with the expected outcome of the screen, since most random mutations introduced to any RNApol sequence are expected to be neutral or deleterious to activity, and a smaller number are expected to increase activity. Random selection is unlikely to produce these drastic and reproducible enrichments for specific amino acid substitutions, which provides support that enrichment is occurring due to increased enzymatic activity.

Templates for this emulsion-based screen are designed so that an initiating RNApol starts transcription, after which the target RNApol sequence is transcribed and translated into target RNApol enzyme that can then produce target transcript itself. Using qPCR, it is possible to track the proportion of transcripts that are produced by the target RNApol after each round. When expressed as a percentage of total transcripts, this gives an approximation of the general level of activity of the pool of target RNApols present in the library. As shown in Table 1, the percentage of transcripts produced by the target RNApol increases from round to round. This indicates that the overall activity level of target RNApols increases as the total rounds of enrichment increase, providing additional support for enrichment of RNApol variants with increased activity.

Table 1: Target transcripts (as a percentage of total transcripts) after each round of enrichment as measured via qPCR.

TABLE 1
Target transcripts (as a percentage of total transcripts) after
each round of enrichment as measured via qPCR.

| | Initial template | TSS | Target transcript (%) | | |
|---|---|---|---|---|---|
| RNApol | SEQ ID NO | | Round 1 | Round 2 | Round 3 |
| RNApol126 | 40 | AT | 0.31 | 1.08 | 3.54 |
| RNApol157 | 39 | AG | 0.53 | 2.62 | 3.52 |
| RNApol157 | 41 | AT | 0.05 | 1.04 | 3.35 |

Secondary Screening and Selection of Hits from In Vitro Screening:

Samples consisting of full-length templates derived from multiple screening rounds of a mutagenized template given in SEQ ID NO: 1 are PCR amplified using the primer sequences given in SEQ ID NO:7 and SEQ ID NO:8. The plasmid given in SEQ ID NO:18 is PCR amplified using the primer sequences given in SEQ ID NO:9 and SEQ ID NO:66, purified from agarose gels after gel electrophoresis, and subjected to DpnI (New England Biolabs, Ipswich, MA) digestion, in which 8 μL of each sample is combined with 1 μL CutSmart buffer (New England Biolabs, Ipswich, MA) and 1 uL DpnI. This mixture is incubated at 37° C. for 30 minutes and then 80° C. for 20 minutes. Insert and plasmid samples are assembled (Gibson 2009, Gibson 2010). Samples are incubated for 1 h at 50° C. Fully assembled plasmids are then transformed into NEB® 5-alpha Competent E. coli (High Efficiency) according to the manufacturer's instructions (New England Biolabs, Ipswich, MA). Resulting colonies are picked into 96-well plates and grown in liquid media. The NUCLEOSPIN® 96 Plasmid kit (Macherey-Nagel, Düren, Germany) is used according to the manufacturer's instructions to isolate plasmids from these cultures.

Isolated plasmids from each clone, as well as plasmids containing each wildtype (WT) RNA polymerase sequence, are subjected to PCR using primers that simultaneously amplify the target RNA polymerase gene with a terminator and append an upstream promoter for a different RNA polymerase initiator. Primers used for PCR amplifying clones derived from the template sequence given in SEQ ID NO:1 are given in SEQ ID NO:7 and SEQ ID NO:8. PCR products are visualized via gel electrophoresis and the NUCLEOSPIN® 96 PCR Clean-up Kit (Macherey-Nagel, Düren, Germany) is used to isolate these amplicons. The QUANT-IT™ dsDNA Broad-Range Assay Kit (Invitrogen Waltham, MA) is used to quantify samples, during which quantitative fluorescence is read on a Synergy HTX Multi-Mode Reader (Biotek, Winooski, VT). A reporter construct is generated from the plasmid sequence given in SEQ ID NO:19 using the primer sequences given in SEQ ID NO:20 and SEQ ID NO:21. Reactions on ice are assembled that consist of 5 ng initiating construct, 17.5 ng reporter construct, Solution A and Solution B from a custom PUREXPRESS® In vitro Protein Synthesis Kit (NEB) that lacks T7 RNA polymerase, RNase inhibitor, and nuclease-free water to 10 μL. Reactions are incubated for 3 h at 30° C. Reactions are halted by mixing with 250 μL of TRIzol and 40 μL of nuclease-free water. Reactions are typically stored at −80° C. at this step. After thawing, 400 μL of ethanol is added. Purification via the DIRECT-ZOL™-96 RNA kit (Zymo, Irvine, CA) is performed as specified by the manufacturer. DNase treatment is performed as described above. RNA samples are then processed via the RNA CLEAN & CONCENTRATOR®-96 Kit (Zymo, Irvine, CA) and eluted in 15 μL nuclease-free water. A 1 μL aliquot of each sample is combined with 0.5 μL of the reverse transcription primer at 10 μM with sequence given in SEQ ID NO:22 and incubated at 70° C. for 5 minutes. Reverse transcription is performed by use of the AFFINITYSCRIPT® cDNA Synthesis Kit (Agilent, Santa Clara, CA) according to the manufacturer's instructions. Control reactions lacking reverse transcriptase are performed for each sample. Quantitative PCR is performed with the primer sequences given in SEQ ID NO:67 and SEQ ID NO:68 on an ARIAMX® Real-Time PCR System (Agilent, Santa Clara, CA) using 1 μL of each sample in 10 μL total reaction volume with the Brilliant III Ultra-Fast SYBR® Green QPCR Master Mix (Agilent, Santa Clara, CA) according to the manufacturer's instructions. Signal from the reporter construct for each clone is compared to signal from the equivalent WT RNA polymerase to narrow hits for further processing.

### Expression, Isolation, and Testing of Variant RNA Polymerases

The variant genes were cloned in an expression plasmid with arabinose inducible promoter and His6 tag. They were sequenced by sanger sequencing and the mutations were identified and mapped to the wild type sequence position. The variant clone plasmid DNA was then transformed into E. coli BL21 strain for expression. The transformants were grown at 30° C. to reach OD₆₀₀ of 0.5-0.7 and induced with 0.025% L-arabinose and incubated at 28° C. for 5 hours. The E. coli cells were pelleted by centrifugation and lysed with sonication and lysozyme. The variant proteins were purified with Ni-affinity purification followed by protein concentration with Amicon MWCOs of 50 kDa filter sold by Millipore (Darmstadt, Germany) and changed into a storage buffer composed of 50 mM Tris pH8.0, 75 mM NaCl, 0.5 mM EDTA and 50% glycerol.

The activity of the variant and the wild-type RNA polymerase was tested in the IVT reaction composed of 40 mM Tris-HCl (pH7.9 at 25° C.), 18 mM Mg²⁺, 10 mM DTT, 2 mM spermidine, 5 mM NTPs, pyrophosphatase 0.002 U/μl, 8 nM DNA template and 0.5 ug enzyme in a 20 μl reaction. The reaction was incubated at 24° C. for 2 hours.

### Example 2: MRNA Sequence Element Discovery by Enrichment within In Vitro Compartments RNA Polymerase Expression and Purification

An open reading frame encoding the initiating RNA polymerase RNApol136 (amino acid sequence given in SEQ ID NO:70) is cloned into a bacterial expression plasmid with an MB 1 plasmid replicon conferring a high copy number in E. coli. The insertion site for the RNA polymerase gene on the plasmid is flanked by an arabinose inducible promoter and a Lambda t1 terminator, allowing for arabinose-inducible expression of the polymerase. The expression construct is sequence verified after cloning. The full sequence of the expression construct for RNApol136 is given in SEQ ID NO:43.

To produce the purified initiating RNA polymerase RNApol136, the RNApol136 expression plasmid is transformed into the E. coli strain BL21 and a single colony picked for cultivation and protein expression. The bacterial cells are grown in LB medium at 37° C. to log phase culture and induced by addition of L-arabinose. After 18 hours of incubation at 15° C., the cultures are harvested by centrifugation and the collected E. coli cells are lysed. RNA polymerase is purified with nickel affinity chromatography according to manufacturer's instructions. The RNA polymerase is eluted with imidazole solution, concentrated with AMICON® Ultra-centrifugal filter sold by Millipore (Darmstadt, Germany) and changed into a storage buffer composed of 50 mM Tris pH8.0, 75 mM NaCl, 0.5 mM EDTA, and 50% glycerol.

### Design of Dual Promoter Plasmid Template for Enrichment of Efficient 5′UTRs:

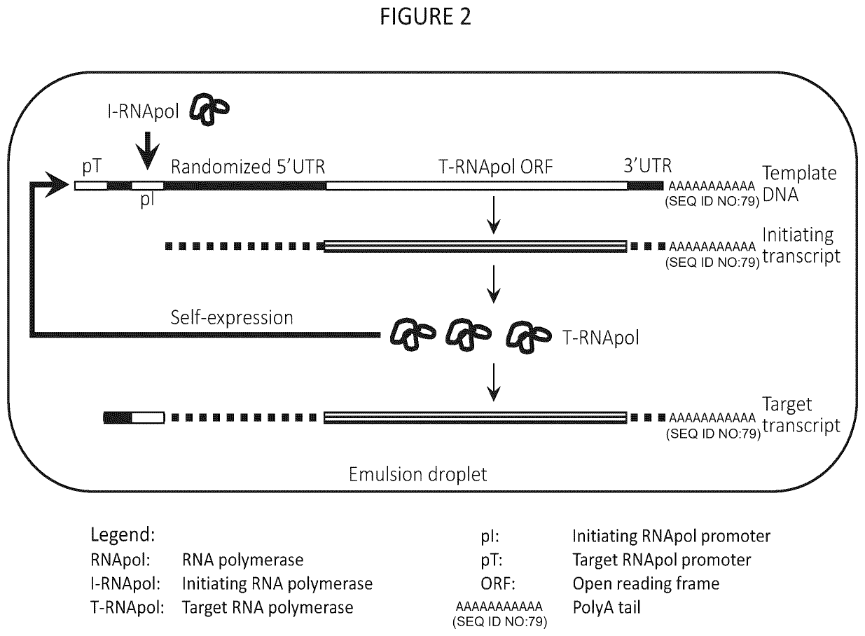

The DNA template incorporated into the in vitro transcription/translation reaction within the emulsion contains the following significant features in order: A promoter and a transcription start site for the target RNA polymerase (RNApol137, given in SEQ ID NO:71), a second promoter and a transcription start site for the initiating RNA polymerase (RNApol136, given in SEQ ID NO:70), and sequence space to randomize a 5′UTR region, ribosome binding site, an open reading frame encoding RNApol137 (codon-optimized for expression in mammalian system), human beta-globin 3′UTR and polyA tail (See FIG. 2). To initiate self-expression exogenously added RNApol136 transcribes the 1^{st} mRNA and translational components express the RNApol137 protein, which acts on the first promoter to produce the 2^{nd} distinct transcript. The distinct and longer transcript produced by RNApol137 allows for selective amplification of RNA molecules with efficient 5′UTR regions. Enrichment is performed over multiple rounds to capture the most efficient 5′UTRs from the library. The full sequence of the DNA template used in this example is given in SEQ ID NO:72 and contains the following sequences elements with the nucleotide positions indicated: 4-26: RNApol137 promoter; 27-28: RNApol137 transcription initiation site; 63-80: RNApol136 promoter; 81-82: RNApol136 transcription initiation site; 90-143: mouse hemoglobin, beta adult major chain (Hbb-b1) 5′UTR; 104-143: portion of 5′UTR being randomized; 144-149: Kozak sequence; 150-2783: RNApol137 ORF (human codon optimized); 2784-2917: human beta-globin 3′UTR; 2918-2937: polyA tail.

### Randomization of the 5′UTR Region to Generate a Screening Library:

A primer containing a 40 nt randomization upstream of the ribosome binding site is obtained from INTEGRATED DNA TECHNOLOGIES® (Coralville, IA): (SEQ ID NO:73; N: 25% A, 25% T, 25% G, 25% C). Thermo Scientific 2× Phire Green Hot Start II PCR Master Mix (Waltham, MA), the plasmid template, the primer containing a 40 nt randomization, and a reverse primer encoding an oligo-dT tail (SEQ ID NO:74), and supplemented magnesium acetate (1.5 mM, final concentration; Alfa Aesar, Haverhill, MA), are used to generate the template library. The PCR product is purified via gel electrophoresis using the NUCLEOSPIN® Gel and PCR Clean-Up kit (Macherey-Nagel, Düren, Germany) and treated with New England Biolabs DpnI enzyme in Smart Cut buffer for 30 min at 37° C. The DpnI-treated DNA library is purified with the NUCLEOSPIN® Gel and PCR Clean-Up kit (Macherey-Nagel, Düren, Germany). Sanger sequencing at ETON Biosciences, Inc. (San Diego, CA) is performed with reverse primer given in SEQ ID NO:75 to confirm randomization.

Self-Expression within In Vitro Compartments in an Emulsion:

An oil-surfactant mixture containing 4.5% Span-80, 0.5% Tween-80, and 95% mineral oil is prepared (all components from Sigma Aldrich, St. Louis, MO) (Miller, 2006). The aqueous reaction mixture is added as five aliquots of 10 μL for 2 minutes to 950 μl oil-surfactant mixture while stirring at 1,500 r.p.m. on ice to create an emulsion. The aqueous reaction mixture consists of the following components from Thermo Scientific 1-Step™ Human Coupled IVT Kit—DNA (Rockford, IL, USA): HeLa cell extract, accessory proteins, and a reactions mixture. Also, 100 ng DNA template, 0.3 micrograms of RNApol136, and nuclease-free water are added to the reacting mix in the described order. After the addition of the final aliquot to the aqueous reaction mixture, stirring is continued for an additional minute. To generate smaller droplets with similar size, samples are stirred on ice for an additional 3 minutes at 8000 r.p.m. on an Ultra-Turrax T10 homogenizer (IKA Works, Inc., Wilmington, NC). The emulsified sample is incubated for 2 h at 30° C. to allow self-expression and enrichment of the most efficient 5′UTRs.

### RNA Isolation and DNA Removal:

To recover RNA transcripts, 50 μl of nuclease water and 500 μl of Trizol are added to the reactions, mixed, and centrifuged at 15,000 g for 5 min at RT. The upper phase, containing the oil mixture, is removed, and the reaming samples mixed before transfer to −80° C. Samples are thawed on ice, and 800 μL of 100% 200-proof ethanol is added to the sample before centrifugation onto silica columns using Zymo Research Direct-zol™ RNA Miniprep Plus kit (Irvine, CA). RNA is recovered using DNAse and RNAse-free water. To remove template DNA, New England Biolabs DNAse I is used with its 10× DNAse I reaction buffer (Ipswich, MA) for 30 min at 37° C. The DNAse step is repeated to ensure the complete removal of DNA. DNA-free RNA is cleaned up using RNA Clean & Concentrator™-5 from Zymo Research (Irvine, CA).

cDNA Synthesis, Amplification of the 5′UTR Region, and Re-Creation of Template:

cDNA synthesis is performed with a primer given in SEQ ID NO:76 that binds downstream of the 5′UTR and igScript™ Reverse Transcriptase from Intact Genomics (San Diego, CA). PCR amplification from the cDNA is conducted with Thermo Scientific 2× Phusion High-Fidelity Master Mix, forward primer given in SEQ ID NO:77 and reverse primer given in SEQ ID NO:78. PCR amplicons containing the 5′UTR and flanking regions are purified via gel electrophoresis using the Nucleospin® Gel and PCR Clean-Up kit (Macherey-Nagel, Düren, Germany). Assembly reaction (Gibson 2009, Gibson 2010) with 5′UTR amplicons and a 3′long fragment (consisting of the RNApol137 gene, human beta-globin 3′UTR, and the polyA tail) is performed. PCR amplification of the assembly with Thermo Scientific 2× Phire Green Hot Start II PCR Master Mix (Waltham, MA) using forward primer given in SEQ ID NO:77 and reverse primer given in SEQ ID NO:74 recreates the full template including the RNApol137 promoter. PCR amplicon is treated with New England Biolabs (Ipswich, MA) DpnI enzyme in Smart Cut buffer for 30 min at 37° C. The DpnI-treated template is purified with the Nucleospin® Gel and PCR Clean-Up kit (Macherey-Nagel, Düren, Germany). Sanger sequencing at ETON Biosciences, Inc. (San Diego, CA) is performed with the reverse primer given in SEQ ID NO:75 to confirm identity.

Selection of Hits from In Vitro Screening and Generation of Templates for Secondary Screening:

To reveal efficient 5′UTR sequences from a screen, the final rounds of enrichment are sequenced at Bio applied Technologies Joint (San Diego, CA) using the MiSeq platform (Illumina, San Diego, CA) and 2×150 bp read length. The 40 nt randomization is mapped to the region upstream of the RBS site using constant 5′ and 3′ flanking regions (10 nt). Top 50 5′UTR variants are incorporated into a template by PCR using Thermo Scientific 2× Phire Green Hot Start II PCR Master Mix (Waltham, MA), forward variant-specific primers ordered from Eurofins Genomics (Louisville, KY) and a reverse primer given in SEQ ID NO:74. The DNA template contains the following significant features in order: RNApol136 promoter and transcription start site and sequence space to introduce the 5′UTR variants, ribosome binding site, a gene encoding firefly luciferase, a mammalian 3′UTR and a 20 bp sequence encoding a polyA tail (See FIG. 2). PCR amplicon is treated with DpnI enzyme in Smart Cut buffer for 30 min at 37° C. (New England Biolabs, Ipswich, MA). The DpnI treated template is purified with the Nucleospin® Gel and PCR Clean-Up kit (Macherey-Nagel, Düren, Germany). Sanger sequencing at ETON Biosciences, Inc. (San Diego, CA) is performed with the reverse primer given in SEQ ID NO:75 to confirm identity.

### Secondary Screen:

50 ng of DNA templates containing 5′UTR variants are used in an in vitro transcription assay with the following components (Final concentrations): 40 mM Tris pH 8.0, 0.6 mM MgCl₂, 5 mM DTT, 2 mM Spermidine, 1 mM of each ribonucleotide (New England Biolabs, Ipswich, MA), and 1 U/μl RNAse inhibitor (New England Biolabs, Ipswich, MA). The reaction is incubated at 30° C. for 60 min. To remove template DNA, New England Biolabs (Ipswich, MA) DNAse I is used with its 10×DNAse I reaction buffer for 30 min at 37° C. The DNAse step is repeated to ensure complete removal of DNA. DNA-free RNA is cleaned up using RNA Clean & Concentrator™-5 from Zymo Research (Irvine, CA) and concentrations are measured on a NanoDrop One instrument from Thermo Scientific (Waltham, MA) and normalized to 200 ng.

400 ng of in vitro transcribed RNA are added to Thermo Scientific 1-Step™ Human Coupled IVT Kit-DNA (Rockford, IL, USA) that includes HeLa cell extract, accessory proteins, the reactions mixture, and nuclease-free water. The reaction is incubated at 30° C. for 0.5, 1, 2, 4, and 6 hours to express luciferase. At each time point, 1 μl of the reaction is transferred to a plate containing substrate reaction buffer, and the luciferase activity is measured on a BioTek Synergy HTX multi-mode microplate reader from Agilent Technologies (Santa Clara, CA). The relative luciferase activity between 5′UTR variants is determined to reveal the top-performing sequence elements from each screen.

### REFERENCES

Abil, Z., & Ellington, A. D. (2018). Compartmentalized Self-Replication for Evolution of a DNA Polymerase. Current Protocols in Chemical Biology, 10(1), 1-17.
Aharoni, A., Amitai, G., Bernath, K., Magdassi, S., & Tawfik, D. S. (2005). High-throughput screening of enzyme libraries: Thiolactonases evolved by fluorescence-activated sorting of single cells in emulsion compartments. Chemistry and Biology, 12(12), 1281-1289.
Agresti, J. J., Kelly, B. T., Jäschke, A., & Griffiths, A. D. (2005). Selection of ribozymes that multiple-turnover Diels-Alder cycloadditions by using in vitro compartmentalization. Proceedings of the National Academy of Sciences of the United States of America, 102(45), 16170-16175.
Aramburu J, Navas-Castillo J, Moreno P, Cambra M (1991). Detection of double-stranded RNA by ELISA and dot immunobinding assay using an antiserum to synthetic polynucleotides. J Virol Methods 33(1-2):1-11.
Arnaud-Barbe N, Cheynet-Sauvion V, Oriol G, Mandrand B, Mallet F (1998). Transcription of RNA templates by T7 RNA polymerase. Nucleic Acids Res. 26(15):3550-3554.
Baiersdörfer M, Boros G, Muramatsu H, Mahiny A, Vlatkovic I, Sahin U, Karikó K (2019). A Facile Method for the Removal of dsRNA Contaminant from In Vitro-Transcribed mRNA. Mol Ther Nucleic Acids 15:26-35.
Bernath, K., Magdassi, S., & Tawfik, D. S. (2005). Directed Evolution of Protein Inhibitors of DNA-nucleases by in Vitro Compartmentalization (IVC) and Nano-droplet Delivery. Journal of Molecular Biology, 345(5), 1015-1026.
Bertschinger, J., & Neri, D. (2004). Covalent DNA display as a novel tool for directed evolution of proteins in vitro. Protein Engineering, Design and Selection, 17(9), 699-707.
Bornscheuer U T, Höhne M, Eds. (2018). Protein Engineering: Methods and Protocols. Methods Mol Biol. 1685. Humana Press, New York, N Y.
Chen, Y., Mandic, J., & Varani, G. (2008). Cell-free selection of RNA-binding proteins using in vitro compartmentalization. Nucleic Acids Research, 36(19), 1-9.
Chen Z, Zeng A P (2016). Protein engineering approaches to chemical biotechnology. Curr Opin Biotechnol. 42:198-205.
Cohen, H. M., Tawfik, D. S., & Griffiths, A. D. (2004). Altering the sequence specificity of HaeIII methyltransferase by directed evolution using in vitro compartmentalization. Protein Engineering Design and Selection, 17(1), 3-11.
Czapinska, H., Siwek, W., Szczepanowski, R. H., Bujnicki, J. M., Bochtler, M., & Skowronek, K. J. (2019). Crystal Structure and Directed Evolution of Specificity of NlaIV Restriction Endonuclease. Journal of Molecular Biology, 431(11), 2082-2094.
Diehl F, Li M, He Y, Kinzler K W, Vogelstein B, Dressman D (2006). BEAMing: single-molecule PCR on microparticles in water-in-oil emulsions. Nat Methods 3(7):551-559.
Di Grandi D, Dayeh D M, Kaur K, Chen Y, Henderson S, Moon Y, Bhowmick A, Ihnat P M, Fu Y, Muthusamy K, Palackal N, Pyles E A (2023). A single-nucleotide resolution capillary gel electrophoresis workflow for poly (A) tail characterization in the development of mRNA therapeutics and vaccines. J Pharm Biomed Anal. 236:115692.
Doi, N., Kumadaki, S., Oishi, Y., Matsumura, N., & Yanagawa, H. (2004). In vitro selection of restriction endonucleases by in vitro compartmentalization. Nucleic Acids Research, 32(12).
Eisenbeis S, Höcker B (2010). Evolutionary mechanism as a template for protein engineering. J Pept Sci. 16(10):538-544.
Fen, C. X., Coomber, D. W., Lane, D. P., & Ghadessy, F. J. (2007). Directed Evolution of p53 Variants with Altered DNA-binding Specificities by In Vitro Compartmentalization. Journal of Molecular Biology, 371(5), 1238-1248.
Foo J L, Ching C B, Chang M W, Leong S S (2012). The imminent role of protein engineering in synthetic biology. Biotechnol Adv. 30(3):541-549.
Gandhi V, O'Brien M H, Yadav S (2020). High-quality and high-yield RNA extraction method from whole human saliva. Biomark Insights. 15:1177271920929705.
Ghadessy F J, Ong J L, Holliger P (2001). Directed evolution of polymerase function by compartmentalized self-replication. Proc Natl Acad Sci USA 98(8):4552-4557.
Ghadessy, F. J., Ramsay, N., Boudsocq, F., Loakes, D., Brown, A., Iwai, S., Vaisman, A., Woodgate, R., & Holliger, P. (2004). Generic expansion of the substrate spectrum of a DNA polymerase by directed evolution. Nature Biotechnology, 22(6), 755-759
Ghadessy F J, Holliger P (2007). Compartmentalized self-replication: a novel method for the directed evolution of polymerases and other enzymes. Methods Mol Biol. 352:237-248.
Gholamalipour Y, Karunanayake Mudiyanselage A, Martin C T (2018). 3′ end additions by T7 RNA polymerase are RNA self-templated, distributive and diverse in character-RNA-Seq analyses. Nucleic Acids Res. 46(18):9253-9263.
Gianella, P., Snapp, E. L., & Levy, M. (2016). An in vitro compartmentalization-based method for the selection of bond-forming enzymes from large libraries. Biotechnology and Bioengineering, 113(8), 1647-1657.
Gibson D G, Young L, Chuang R Y, Venter J C, Hutchison C A 3rd, Smith H O (2009). Enzymatic assembly of DNA molecules up to several hundred kilobases. Nat Methods. 6(5):343-345.
Gibson D G, Smith H O, Hutchison C A 3rd, Venter J C, Merryman C. (2010). Chemical synthesis of the mouse mitochondrial genome. Nat Methods. 7(11):901-903.
Griffiths, A. D., & Tawfik, D. S. (1998). Man-made cell-like compartments for molecular evolution. Nature Biotechnology, 16 (July), 652-656.
Griffiths, A. D. (2003). Directed evolution of an extremely fast phosphotriesterase by in vitro compartmentalization. The EMBO Journal, 22(1), 24-35.
Griffiths A D, Tawfik D S (2006). Miniaturising the laboratory in emulsion droplets. Trends Biotechnol. 24(9):395-402.
Gunter H M, Idrisoglu S, Singh S, Han D J, Ariens E, Peters J R, Wong T, Cheetham S W, Xu J, Rai S K, Feldman R, Herbert A, Marcellin E, Tropee R, Munro T, Mercer T R (2023). mRNA vaccine quality analysis using RNA sequencing. Nat Commun. 14(1):5663.
Hadi M, Stacy E A (2023). An optimized RNA extraction method for diverse leaves of Hawaiian Metrosideros, a hypervariable tree species complex. Appl Plant Sci. 11(3):e11518.
Henderson J M, Ujita A, Hill E, Yousif-Rosales S, Smith C, Ko N, McReynolds T, Cabral C R, Escamilla-Powers J R, Houston M E (2021). Cap 1 messenger RNA synthesis with co-transcriptional CleanCap® analog by In Vitro Transcription. Curr Protoc. 1(2):e39.
Hornung V, Ellegast J, Kim S, Brzozka K, Jung A, Kato H, Poeck H, Akira S, Conzelmann K K, Schlee M (2006). 5′-Triphosphate RNA is the ligand for RIG-I. Science. 314(5801):994-997.
Houlihan, G., Gatti-Lafranconi, P., Kaltenbach, M., Lowe, D., & Hollfelder, F. (2014). An experimental framework for improved selection of binding proteins using SNAP display. Journal of Immunological Methods, 405, 47-56.
Johnson L B, Huber T R, Snow C D (2014). Methods for library-scale computational protein design. Methods Mol Biol. 1216:129-59.
Kaushik M, Sinha P, Jaiswal P, Mahendru S, Roy K, Kukreti S (2016). Protein engineering and de novo designing of a biocatalyst. J Mol Recognit. 29(10):499-503.
Karikó K, Muramatsu H, Ludwig J, Weissman D (2011). Generating the optimal mRNA for therapy: HPLC purification eliminates immune activation and improves translation of nucleoside-modified, protein-encoding mRNA. Nucl. Acids Res. 39(21), e142.
Körfer, G., Besirlioglu, V., Davari, M. D., Martinez, R., Vojcic, L., & Schwaneberg, U. (2022). Combinatorial In VitroFlow-assisted mutagenesis (CombIMut) yields a 41-fold improved CelA2 cellulase. In Biotechnology and Bioengineering.
Leatherbarrow R J, Fersht A R (1986). Protein engineering. Protein Eng. 1(1):7-16.
Lee, Y. F., Tawfik, D. S., & Griffiths, A. D. (2002). Investigating the target recognition of DNA cytosine-5 methyltransferase Hhal by library selection using in vitro compartmentalisation. Nucleic Acids Research, 30(22), 4937-4944
Leisola M, Turunen O (2007). Protein engineering: opportunities and challenges. Appl Microbiol Biotechnol. 75(6):1225-1232.
Levy, M., Griswold, K. E., & Ellington, A. D. (2005). Direct selection of trans-acting ligase ribozymes by in vitro compartmentalization. Rna, 11(10), 1555-1562.
Levy, M., & Ellington, A. D. (2008). Directed Evolution of Streptavidin Variants Using In Vitro Compartmentalization. Chemistry and Biology, 15(9), 979-989.
Lu, W.-C., Levy, M., Kincaid, R., & Ellington, A. D. (2014). Directed evolution of the substrate specificity of biotin ligase. Biotechnology and Bioengineering, 111(6), 1071-1081.
Lutz S, Benkovic S J (2000). Homology-independent protein engineering. Curr Opin Biotechnol. 11(4):319-324.
Lutz S, Iamurri S M (2018). Protein Engineering: Past, Present, and Future. Methods Mol Biol. 1685:1-12.
Ma, F., Xie, Y., Huang, C., Feng, Y., & Yang, G. (2014). An improved single cell ultrahigh throughput screening method based on in vitro compartmentalization. PLOS ONE, 9(2), 1-10.
Marcheschi R J, Gronenberg L S, Liao J C (2013). Protein engineering for metabolic engineering: current and next-generation tools. Biotechnol J. 8(5):545-55.
Mastrobattista, E., Taly, V., Chanudet, E., Treacy, P., Kelly, B. T., & Griffiths, A. D. (2005). High-throughput screening of enzyme libraries: In vitro evolution of a β-galactosidase by fluorescence-activated sorting of double emulsions. Chemistry and Biology, 12(12), 1291-1300.
Miller O J, Bernath K, Agresti J J, Amitai G, Kelly B T, Mastrobattista E, Taly V, Magdassi S, Tawfik D S, Griffiths A D (2006). Directed evolution by in vitro compartmentalization. Nat Methods 3(7):561-570.
Mu X, Greenwald E, Ahmad S, Hur S (2018). An origin of the immunogenicity of in vitro transcribed RNA. Nucleic Acids Res. 46(10):5239-5249.
O'Fágáin C. Engineering protein stability (2011). Methods Mol Biol. 681:103-36.
Ostafe, R., Prodanovic, R., Nazor, J., & Fischer, R. (2014). Ultra-high-throughput screening method for the directed evolution of glucose oxidase. Chemistry and Biology, 21(3), 414-421.
Packer M S, Liu D R (2015). Methods for the directed evolution of proteins. Nat Rev Genet. 16(7):379-394.
Paegel, B. M., & Joyce, G. F. (2010). Microfluidic compartmentalized directed evolution. 14th International Conference on Miniaturized Systems for Chemistry and Life Sciences 2010, MicroTAS 2010, 1(7), 307-308.
Poveda C, Biter A B, Bottazzi M E, Strych U (2019). Establishing preferred product characterization for the evaluation of RNA vaccine antigens. Vaccines 7(4), 131.
Prodanovic, R., Ostafe, R., Scacioc, A., & Schwaneberg, U. (2011). Ultrahigh Throughput Screening System for Directed Glucose Oxidase Evolution in Yeast Cells. Combinatorial Chemistry & High Throughput Screening, 14(1), 55-60.
Rueden C T, Schindelin J, Hiner M C, DeZonia B E, Walter A E, Arena E T, Eliceiri K W (2017). ImageJ2: ImageJ for the next generation of scientific image data. BMC Bioinformatics, 18(1). doi:10.1186/s12859-017-1934-z
Ryckelynck, M., Baudrey, S., Rick, C., Marin, A., Coldren, F., Westhof, E., & Griffiths, A. D. (2015). Using droplet-based microfluidics to improve the catalytic properties of RNA under multiple-turnover conditions. Rna, 21(3), 458-469.
Sakatani, Y., Mizuuchi, R., & Ichihashi, N. (2019). In vitro evolution of phi29 DNA polymerases through compartmentalized gene expression and rolling-circle replication. Protein Engineering, Design & Selection: PEDS, 32(11), 481-487.
Schindelin J, Arganda-Carreras I, Frise E, Kaynig V, Longair M, Pietzsch T, Cardona A (2012). Fiji: an open-source platform for biological-image analysis. Nature Methods, 9(7), 676-682.
Schneider C A, Rasband W S, Eliceiri, K W (2012). NIH Image to ImageJ: 25 years of image analysis. Nature Methods, 9(7), 671-675.
Schönborn J, Oberstrass J, Breyel E, Tittgen J, Schumacher J, Lukacs N (1991). Monoclonal antibodies to double-stranded RNA as probes of RNA structure in crude nucleic acid extracts. Nucleic Acids Res. 19(11):2993-3000.
Sepp, A., & Choo, Y. (2005). Cell-free Selection of Zinc Finger DNA-binding Proteins Using In Vitro Compartmentalization. Journal of Molecular Biology, 354(2), 212-219.
Shin H, Cho B K (2015). Rational Protein Engineering Guided by Deep Mutational Scanning. Int J Mol Sci. 16(9):23094-23110.
Singh R K, Lee J K, Selvaraj C, Singh R, Li J, Kim S Y, Kalia V C (2018). Protein Engineering Approaches in the Post-Genomic Era. Curr Protein Pept Sci. 19(1):5-15.
Sinha R, Shukla P (2019). Current Trends in Protein Engineering: Updates and Progress. Curr Protein Pept Sci. 20(5):398-407.
Son, K N, Liang, Z G, and Lipton, H L (2015). Double-stranded RNA is detected by immunofluorescence analysis in RNA and DNA virus infections, including those by negative-stranded RNA viruses. J. Virol. 89(18), 9383-9392.
Stapleton, J. A., & Swartz, J. R. (2010). Development of an in vitro compartmentalization screen for high-throughput directed evolution of [FeFe] hydrogenases. PLOS ONE, 5(12), 1-8.
Swint-Kruse L (2016). Using Evolution to Guide Protein Engineering: The Devil IS in the Details. Biophys J. 111(1):10-18.
Takeuchi R, Choi M, Stoddard B L (2014). Redesign of extensive protein-DNA interfaces of meganucleases using iterative cycles of in vitro compartmentalization. Proc Natl Acad Sci USA. 111(11):4061-4066.
Tawfik D S, Griffiths A D (1998). Man-made cell-like compartments for molecular evolution. Nature Biotechnol. 16(7):652-656.
Tay, Y., Ho, C., Drooge, P., & Ghadessy, F. J. (2009). Selection of bacteriophage λ integrases with altered recombination specificity by in vitro compartmentalization. Nucleic Acids Research, 38(4).
Tay Y, Ho C, Droge P, Ghadessy F J (2010). Selection of bacteriophage lambda integrases with altered recombination specificity by in vitro compartmentalization. Nucleic Acids Res. 38(4):e25.
Tran, D. T., Cavett, V. J., Dang, V. Q., Torres, H. L., & Paegel, B. M. (2016).
Evolution of a mass spectrometry-grade protease with PTM-directed specificity. Proceedings of the National Academy of Sciences of the United States of America, 113(51), 14686-14691.
Tu, R., Martinez, R., Prodanovic, R., Klein, M., & Schwaneberg, U. (2011). A flow cytometry-based screening system for directed evolution of proteases. Journal of Biomolecular Screening, 16(3), 285-294.
Tu Y, Das A, Redwood-Sawyerr C, Polizzi K M (2024). Capped or uncapped? Techniques to assess the quality of mRNA molecules. Curr. Opin. Systems Biol. 37:100503.
Uyeda, A., Watanabe, T., Kato, Y., Watanabe, H., Yomo, T., Hohsaka, T., & Matsuura, T. (2015). Liposome-Based in Vitro Evolution of Aminoacyl-tRNA Synthetase for Enhanced Pyrrolysine Derivative Incorporation. ChemBioChem, 16(12), 1797-1802.
Warzak D A, Pike W A, Luttgeharm K D (2023). Capillary electrophoresis methods for determining the IVT mRNA critical quality attributes of size and purity. SLAS Technol. 28(5):369-374.
Wilding M, Hong N, Spence M, Buckle A M, Jackson C J (2019). Protein engineering: the potential of remote mutations. Biochem Soc Trans. 47(2):701-711.
Woodley J M (2013). Protein engineering of enzymes for process applications. Curr Opin Chem Biol. 17(2):310-316.
Wrenbeck E E, Faber M S, Whitehead T A (2017). Deep sequencing methods for protein engineering and design. Curr Opin Struct Biol. 45:36-44.
Yang K K, Wu Z, Arnold F H (2019). Machine-learning-guided directed evolution for protein engineering. Nat Methods 16(8):687-694.
Zaher, H. S., & Unrau, P. J. (2007). Selection of an improved RNA polymerase ribozyme with superior extension and fidelity. Rna, 13(7), 1017-1026
Zawaira A, Pooran A, Barichievy S, Chopera D (2012). A discussion of molecular biology methods for protein engineering. Mol Biotechnol. 51(1):67-102.
Zheng, Y., & Roberts, R. J. (2007). Selection of restriction endonucleases using artificial cells. Nucleic Acids Research, 35(11).
Zoller M J (1991). New molecular biology methods for protein engineering. Curr Opin Biotechnol. 2(4):526-531.

We claim:

1. A screening system for isolating nucleic acid sequences encoding RNA polymerases with altered properties compared to a parental RNA polymerase, containing one or more nucleic acid templates comprising:
(a) An RNA polymerase promoter; and
(b) A sequence encoding an RNA polymerase that recognizes the promoter in part (a), placed downstream of the promoter in part (a) such that the encoded RNA polymerase can transcribe the sequences encoding it.

2. A process for improving RNA polymerase activity comprising:
(a) Creating a collection of in vitro compartments containing a mixture of:
i) a collection of nucleic acid templates encoding RNA polymerases with altered properties compared to a parental RNA polymerase which recognizes a first promoter present on the nucleic acid templates;
ii) an initiating RNA polymerase that recognizes a second promoter present on the nucleic acid templates of (i);
iii) proteins, nucleic acids and other reaction components necessary for in vitro transcription and translation;
(b) Incubating the mixture of step (a) under conditions in which the RNA polymerases encoded in the nucleic acid templates produce RNA transcripts encoding RNA polymerases;
(c) Isolating the RNA transcripts produced in step (b) and processing the isolated RNA transcripts to regenerate nucleic acid templates encoding RNA polymerases; and
(d) Repeating steps (a)-(c) to enrich for nucleic acid template molecules encoding RNA polymerases with altered activity or properties compared to a parental RNA polymerase.

3. The process for improving RNA polymerase activity of claim 2, wherein the altered property of the RNA polymerase is higher RNA yield in in vitro transcription reactions.

4. The process for improving RNA polymerase activity of claim 2, wherein the altered property of the RNA polymerase is higher integrity of RNA synthesized in in vitro transcription reactions.

5. The process for improving RNA polymerase activity of claim 2, wherein the altered property of the RNA polymerase is lower amounts of double-stranded RNA in RNA synthesized in in vitro transcription reactions.

6. The process for improving RNA polymerase activity of claim 2, wherein the altered property of the RNA polymerase is lower amounts of short or truncated transcripts in RNA synthesized in in vitro transcription reactions.

7. The process for improving RNA polymerase activity of claim 2, wherein the altered property of the RNA polymerase is higher fidelity.

8. The process for improving RNA polymerase activity of claim 2, wherein the altered property of the RNA polymerase is higher capping efficiency.

9. The process for improving RNA polymerase activity of claim 2, wherein the altered property of the RNA polymerase is higher cap incorporation efficiency.

10. The process for improving RNA polymerase activity of claim 2, wherein the altered property of the RNA polymerase is the ability to use a specific transcription start site.

11. The process for improving RNA polymerase activity of claim 2, wherein the altered property of the RNA polymerase is increased polyA sequence length or uniformity.

12. The process for improving RNA polymerase activity of claim 2, wherein the altered property of the RNA polymerase is RNA synthesis at a specific temperature.

13. The process for improving RNA polymerase activity of claim 2, wherein the altered property of the RNA polymerase is higher protein stability.

14. The process for improving RNA polymerase activity of claim 2, wherein the altered property of the RNA polymerase is RNA synthesis in the presence of salts or other molecules that inhibit RNA synthesis by the RNA polymerase.

15. The process for improving RNA polymerase activity of claim 2, wherein the altered property of the RNA polymerase is the generation of monophosphates, diphosphates or triphosphates at the transcriptional 5′ end.

16. A nucleic acid molecule comprising:
(a) Two promoter sequences recognized by RNA polymerases; and
(b) A sequence encoding an RNA polymerase that recognizes one of the promoters in part (a),
wherein each of the two promoter sequences in part (a) directs transcription of the sequence encoding the RNA polymerase of (b).

17. A screening system for isolating nucleic acid sequences comprising:
(a) The nucleic acid molecule of claim 3; and
(b) A system of in vitro compartmentalization separating two or more reactions into separate compartments within the same sample.

18. The screening system of claim 17, wherein untranslated sequences are isolated which, when operably linked to a coding sequence, influence translation of the coding sequence.

19. The screening system of claim 18, wherein the untranslated sequence has utility as a 5′ untranslated region.

20. The screening system of claim 18, wherein the untranslated sequence has utility as a 3′ untranslated region.

21. The screening system of claim 18, wherein the untranslated sequence has utility as a polyA sequence.

22. The screening system of claim 18, wherein the untranslated sequence has utility as a Kozak sequence or ribosome binding site.

23. The screening system of claim 18, wherein the untranslated sequence has utility as an internal ribosome entry site.

24. A process for isolating promoter sequences recognized by an RNA polymerase, comprising:
(a) Creating a collection of in vitro compartments containing a mixture of:
i) An initiating RNA polymerase;
ii) a collection of nucleic acid templates encoding a second RNA polymerase distinct from the initiating RNA polymerase, a first promoter recognized by the encoded RNA polymerase, and diverse sequences upstream of the RNA polymerase coding sequence that serve as the source of promoter elements for the initiating RNA polymerase of (i);
iii) proteins, nucleic acids and other reaction components necessary for in vitro transcription and translation;
(b) Incubating the mixture of step (a) under conditions in which the RNA polymerases encoded in the nucleic acid templates produce RNA transcripts encoding RNA polymerases;
(c) Isolating the RNA transcripts produced in step (b) and processing the isolated RNA transcripts to regenerate nucleic acid templates encoding RNA polymerases; and
(d) Repeating steps (a)-(c) to enrich for nucleic acid template molecules encoding promoter sequences recognized by the initiating RNA polymerase.

25. A process for improving RNA polymerase coding sequences comprising:
(a) Creating a collection of in vitro compartments containing a mixture of:
i) a collection of nucleic acid templates of different sequences encoding the same RNA polymerase which recognizes a first promoter present on the nucleic acid templates;
ii) an initiating RNA polymerase that recognizes a second promoter present on the nucleic acid templates of (i);
iii) proteins, nucleic acids and other reaction components necessary for in vitro transcription and translation;
(b) Incubating the mixture of step (a) under conditions in which the RNA polymerases encoded in the nucleic acid templates produce RNA transcripts encoding RNA polymerases;
(c) Isolating the RNA transcripts produced in step (b) and processing the isolated RNA transcripts to regenerate nucleic acid templates encoding RNA polymerases; and
(d) Repeating steps (a)-(c) to enrich for nucleic acid template molecules encoding RNA polymerases transcribed or translated at higher levels than a specific reference sequence encoding the RNA polymerase.


SEQUENCE LISTING (78 sequences)

SEQ ID NO: 1 - DNA, 2893 bp, synthetic construct
  misc_feature 1..2893: note = Synthetic: F2270, Screening template for RNApol137
  source 1..2893: mol_type = other DNA
agatctcgatcccgcgaaattaggcactacaataggagaagctgaaagtaacaacggtttcactctagaaattgggccactatagcaacaccaaaaccatcatcaggaggaattaaccatgcatcatcatcaccatcacggcagctacactagcgatgagctttaccagaaacaacttgagctagaaggggagatgcatggctacggtgttactcgctttgaccgtaacaaccaacgtgcgattgatagcggtacaccttcagacactgattggaaccgccgtttactttctaacttcattgagcctatggtgaagggtattaatgcttataaggagtactacaagactaaagctggacggcctacagtggctttaaagtacatccggcaggtgcagccagagcaagcggcatacatcgccattaagaacattctggatgtgctgggtagtacaacatttgatgctaactggcttgttaccactattgggcgtaggattgaagaccaagtaagatttactaagcttgaggaggctgcacctaagtatgtaggcaaggttaaggatagcttagctaagcgcaactctctccagtatgcacaccagcataaggtactggtcgctactgagaagaagctagcggagaatccagaccgtgctcagcttgagctgccacgctggcaggagtgggctgaggaggattgcaagcacgttggtagcttgctggtcaatatctttgagcagtgtattctgtttgagggtgagccagtaatccgcaaggaggtgcagacagttcgcaaaggtacactggtcttcatcaagcctactgagaaagttaccaagtggattcatgagttcagagaggctattggcgggcttgctccagcttacgcaccttgcgtagttcctccgcttgattggacttccccatttactggcgggttccatactgaagctgttagcagcactctacatcttgctaaggttcgcaacaagcgccacctgcgcaagctgactaaagagcagatgccagcagtctacaaggctgtaaataacttgcagaaagtgcgctggcgtatctctgagcgtgtactggcaaccgctaatacactagtggagctgggcttgccttatgcgctgccgtcaaaggatgaaagcgactggaaagaaaagaatccatgccctgtacctgagtacctgcaagacctgcgaggcgagcagctgaaagcggcgcttactgccagccagtgggaggcttttcaagagtggaaacaactggcacgccagaactacgacgaggagagcgagcgcgtagcaagctttagggaagttgttcgtactttagggcaggctaaccgctacgttggctttgatgcaatctactttgtgtacacgctggactttcgcgggcgtgtctactgccagagcagccttgtaagcccacagggtggcgacttgcagaaagccctcatcaagtttgcggatggtatgaagctgggtgagcgcggcgagtactggttcaaggtgcatggcgctaacgaatggggctgggataagaagacctttgatgagcgcgtagctctggtcagcgaacctgagttctgcgagatgtgcctagacattgccagtgacccagtgaccttcaatgactggattaaggccgataagccgtggcagttcctcaactggtgctttgagtatgctgacttcctgaagcatgtgcaatcagctggcaacccacaggactttgtaagctatatcccttgtgcaatggatggttcttgctctggcatccagcactactctgcaatgctgcgcgatactgttggcggtactgctgttaacttggttaactctgacaagcctaatgacatctacggagaggtctgcaaggtgactattaaagagctgcaagcaattgctgacggctcacaggtatatgacagcaagattgaccctgtattagctcagcagctggcgcaggagtggctacgtctgcaaccaaatcgcagcctgaccaagaaacctgttatgaccctgccttacggcagcactcagttaacctgccgtgagcatgttagccagtggcttaaagatttgcagaaggaggagaacaagcgagctaaggcagagttcagagagcctatgaaggtacacgctttcggcgatgcagacagcgctatgccgctaaagtttgctgagtctcttatgactagcattgtttggcactctatcggcaaggttgtagtagctgcgagggctggtatggcttacatcaaagccgtaacaagctcagtggctaagatgaatatgccgcttgagtggactactccaacaggctttattgtccgtcaggagatttaccagttcacaacacgtcaggtaaatactcagctgctgggtggaactaagtttgtggtcagcactaagagtaaagatattgattatcatcgtatgattaactcctgtgcgcctaactttgttcactcaatggatgcgagccaccttacactggcaactaactactttgcagatgcaggtattagcagcattgcagtaatccatgatagctttggcacacatgcaggagccactgacctgctgcgtgagcgtctgcgagcttcaatggtagatatgtacgaacagcatgacgttatcactaacttccttgctgaaactgagggcagacttatgacagcttttgagcacattcgagtgccagagcgtggtgagctgcaacttgatagcattaatcaatctacatacgcttttgcgtgatgcacgtgaggatccaactcgagaacttagatggtattagtgacctgtaacagagcattagcgcaaggtgatttttgtcttcttgcgctaattttttgtcatcaaacctgtcgctag

SEQ ID NO: 2 - DNA, 2671 bp, synthetic construct
  misc_feature 1..2671: note = Synthetic: F2272, Screening template for RNApol031
  source 1..2671: mol_type = other DNA
agatctcgatcccgcgaaatattgggccactatagcaacaccaaaaccatcatcacaacggtttcactctagaataggcactacaataggagaagctgaaagtaaggaggaattaaccatgcatcatcatcaccatcacggcagcaatcaggcggaacttgagcgggaaatggcagacggcggtcgccgtgcggcaatggcaaggtttgaaaccgccgagtccgcgaacgacgcagcgagtaacccatatgccgccgcgatcttccgccgattcatcaagcctctcagcgaggggcttgacgtgtaccttcacaaagcagtccgaggcgtagcagcgaaatcaaaggtgctgctgcgcggacaggacacgctggcgttagcgtacatcacgatccgtggtgtgattaacgcaagcctctccgaaggggaggcagtatttgctggtgtggccatggagattggccgcactgtgtacagcgaggtggctctcaggcaattcgaagacctgaacccggagctgtattacacgctggtgcaagaccttgaacgccgcatgaccaagagcgagcgtcaccggttcaacgtgatgcgatccagtgccgagaaggacggaacgccgttgccggtgtgggagccagcgctcaagatggacgtgggtacgttgttgctgggcgcaagccttgacatcgggctgatcgaaacctttgatctgcgcatgaagggcaagacaaccaagcacttgaggctgcacccggatctgctggagctgatcgagcagattaaggggttcgtggccggtgcttcgccgttcaatctgccgtgcgtcgagccgccgaaaccatgggttacgcctatggacggtggctggcacacgaatgcgatgcgccggacgctgccttgcatggtacgcggacaggcatcggcaacgattgaggacgtaggcccgcgagttctcaaggcactgaacaaggtgcagagcgttgcgtgggaggtgaacgagcgaattcttgaggtagcggagttcgcaagggagcattttgacgtacaggacgtgctggttagcgataagcgcggcgggatgccggacaagcctctgtttatgcaggctgatcccgacctcaaaatcgttgacatgaacgaattcgaactggcacagttcactgaatggcgacaggaggcacgagaatggcacactcagcagaaggtacggggagcgcatgcggggagtacgaacgaagctatccgcgtggcgaacaagtacaaggggaacccgctctggtttgtctattctgtggactatcggagccgattctatgcctctggacaggggctctccccacaagggaacgacctttcaaaagcgctcatccactttgcaaagtctgcacctatacgaacttccgctggcctcttctggttccgggtagccggagctaacaagtgggccgttgacaaactagacaagaagccgctcgatgttcgggcgcaatgggtgatagacaatgaagagtttatcagtcggatcgctgatgacccgatcagccaccgagaatggacagatgctgacgtacctttccagtttcttgcgtggtgctttgaatatgcagcttggcggaaagaccctgaatcgtttcgcactcgaatcccacttggacaagacggcagttgtaatggtcttcaacacttctcagcaatgcttagagatagggttggcgggcgagcaaccaacctcattcccgataccgtgcaacacgacatctatggcctcgtcgcaacagcaactgctggcatcgtgcaagcggacattgacgaatgcgatattgccaagcgctggaagtcacacgaactttcgcggtcgttggttaagcggtcagtaatgaccctgccgtatggctccacacggcactcctgccgcgacttcatcatgaaggagtacatggacaaggggagtgcgccggagttcgacaagcgcgagaatgagcctgctgcgcggtggttgtcgtaccgggtatgggacggcatcggtgaggtggtcgtgaagggccgacaggcgatggagtggttgcaggcagcatcggccatcatgtgcgccgggaacgccccacacatcgaatggaggaaccctgcgggctttctggtacggcagcgttaccacgcccgggaaatgctcagggtgagctgtcacagcctctcaggcaagcgtatccgcatcaacgtccagaccttccgggacgagggcgatccacggcggcaccggaacggcatcgcgccgaacttcgtgcatagctgtgacgcggctcacctgacgttcttcacgaacgactgggaggacgaggacggcggcgatctggcactcgtccacgatgactacggcgcactggccgacgaggtgcccaagctgcacagaatcctgcgtaggtcgtttgtggacatgtacctgtaccatgaccctctgaaagcgcttgcggcgcaggtggagggcttaccggagcttcctgagtccggggatctagatcttgaggaagtaaaccggtcggtgtacttcttctgttaatgcacgtgaggatccaactcgagaacttagatggtattagtgacctgtaacagagcattagcgcaaggtgatttttgtcttcttgcgctaattttttgtcatcaaacctgtcgctag

SEQ ID NO: 3 - DNA, 2689 bp, synthetic construct
  misc_feature 1..2689: note = Synthetic: F2274, Screening template for RNApol002
  source 1..2689: mol_type = other DNA
agatctcgatcccgcgaaattcgaccctattaaacaaggagccaattacaacggtttcactctagaaattgggccactatagcaacaccaaaaccatcatcaggaggaattaaccatgcatcatcatcaccatcacggcagcattacgctggaacagcagttagcatgggaacacaagcatcgtgagttgggccgtgataaggtcatggagagtctgcgtaaggcagaagaacagggccgagttactgataccccgttaggtacgggtgtccttcggaagtacatcatgtggttgtcccagcgtatcgctactgacctgactgtggacctgggtaaaccaggccgcagcaaggctcactcgccgctactgaaagacctggacccggacagtgttgcactgattactatctcatctgttattaacacgttgagccaggataatcggggactgtctgccgtggctatgcagattgggaagaccatctacggtgaactggcgctgtgctacttccgggacatgaaggaagacctgtacgaagctatggtccacgacctcactaagaagatgtccaaagacctgcgccacaggctcacagtgatgcgtatgcaagctgagaaggctggcgtatcaatacctgagtggacgccatcacagaagctacaggtaggtctgtatctgttaagcctcattgacggtgaggatggcctggttgagcagtacactgacttcactaacaagaagacaacgtacaagctacagcttagacctgctgtaatgcagctaatgaacagcgcagaatcaagcatactggcccacgctggcttcgcagcaccttgcctgatacaaccacaggattgggacggcgatggcgtaggcggcttctacggcgatttaaagatacgtgctccccgcttcttcaagggtgacagctaccaaatggaggtaatgaagaagctgggttgtgatttacgtgtggtgttgggtatgttaaatgcgcaccagtcggtagcgtggaaggtaaacccattcatcctggaccttgtgaagggtatgcgcctccgtggatacgagactgatgacgttatgttcaccagtgctcacccggaaccaccaaggccactcttcctagacctggctgacaagataggcatgaccgttccgcaggaagttgagttcaaacaatggaagcagaccaagcgtgactggcatacgaagattcgactggtgagcagggcagaagccaagctatcacgagtcatctatgccgctcaagacatgcttgaatactctgagttcttctttgtgttccagaccgacgaccgtggcaggatgtatccggtatccggcccactgaatccccagggcagcgacatgcagaaggcgatgttacacgctgcgcatggcgagccaatagcggataaggtggctgagaaatggttcaagctgaatctggctactaagtatggagttgacaaactcaacattgaccagtgcgtacagtggactattgacaaccacgacaacatcatgcgtgcagcagaagacccgctgaatcgtgatgcgttctactggtggtcagaagcggataagccgctccagttcattgcattgtgtgatgagtaccgacgctaccacctggacccggttaacttcgtggcccggatagcggtagcaatggatggcacctgtaacggcctacagaactactctgcgctgttacgtgatgaggtgggtggtagggcgactaacctcattggtgaggatggtgacgctcccaacgacatctacggtgatgtggcggtcgctgccgagaagcgattgcagtatgcggaagagtgtgctgctaaattagcctggctaaaggagggcttcaaccgtgggcttactaagaaatcagtaatgacccaggtgtatggctcaacctttggcacatgccgtaagtcaatagtggcttactgtgtggagaaggcgctgtttgaagacaaggaacgctgggagttcgctgactttgccagtcatctggtatgggaaggcatcggtgacgtagtggtcaaaggtaaagaagcaatggattggttgcgtaaagtatccggctgtgctatgaagactggagacgactacatttcctggcctgcaccaagcggtttccgggtagttcaggtatatcgtaagtcagaagtcaagcgggtacaggctcaagtcggtaataaaatcaccttgcgtcttcgtgaagaaactgatttacctgataagattcgccataggaacgccacacctcctaactttattcactctgtggatgctagtcacatggcattcgtatcagtgaggatgtacctgatgttccctggcctgttcatgcacatgattcacgatgacttcggtgcattgccgagtaaggcacaggctctgtatgacactatccgtagtgagtttgttcagatgcacgagaattattctcttgactgtttcgtatctgagtatggtcatcgtgtgaaattaccacctgttcctgagcgtggttcactggacattcgctgtgtgttaacctcaccgaatttcttccgctaatgcacgtgaggatccaactcgagaacttagatggtattagtgacctgtaacagagcattagcgcaaggtgatttttgtcttcttgcgctaattttttgtcatcaaacctgtcgctag

SEQ ID NO: 4 - DNA, 2904 bp, synthetic construct
  misc_feature 1..2904: note = Synthetic: F2276, Screening template for RNApol126
  source 1..2904: mol_type = other DNA
agatctcgatcccgcgaaattaattagagaccactatagaagagataaattttaatggcaattcagcacaacggtttcactctagaaattgggccactatagcaacaccaaaaccatcatcaggaggaattaaccatgcatcatcatcaccatcacggcagcgatttacaagaaattcaattacagttagagaatgaaatgtttaacggtggcattcgtagattcgaagcagaccaacaaagacatttacaggcaggtaatgcttctgatactgcatggaatcgtaagcttatctctgagtttatcgcacctatggcacaaggtattcaggcatataaagaagagtacaaaggcaagataggtcgtgctcctcgtgctttagctttcctaaattgtgtagaaaatgaagtgtctgcatatattactatgaaggtagtaatggatatgatacagacagacgtcactttacaatcaatagctatgagtatagctgaccgtattgaagaccaagttagattttctaacttagaaggcaaagctaagaaatactttgagaaagtaaaagcaagtcttaaagcttcacgctctaaacaatatgcacatggtcataaagttatggtagtagctgaaaagaatcttgctgagaagagcgaagatatagaccgttggataccttggggtaaagaggaattactaaatattggtcttactttacttgagatattagaaaactctgtgttctttaatggtgaacctgtcttcttccgtactattcgcactatgggttataagaaaacattatatttcttacagacatctgagaatattggtgaatgggtacgtgcatttaaagaccatgtagcgcaactaagccctgcatatgcaccttgtgttgttcctcctcgtccttggaaatctccttttaatggtggcttccatactgagaaagtagctagtcgcattcgtcttgttaaaggtgaccgcgagcatgtacgtaagttaacacaaaagcagatgccgaaagtgtacaaagctattaatgctttacaaaatacacaatggcaagttaacaaagatattctaaacgtagctgatgaggttgtaagattagaccttggttatggtgtgccacacttcaaacctttaattgataaagataacaagccagctaatcctgtgcctgttgaattccagcacctaagaggtagagagttaaaagaagtgttaagtgatgagcaatggcaagcctttatccactggaaaggtgaatgtgccaagctatacacagcagaaactaagagaggttctaaatctgcttcggtggttagaatgttaggacaagcgcgcaaatatagcatgtttgatgcaatatactttgtatatgctatggattcacgttctcgcgtatacgctcagagtagcacgctaagcccacaaagcgacgacttaggtaaatcattattgagattcacagaagggcgtagcattaattctgtggaagatttgaagtggttctgcattaatggtgctaacctttggggttgggataagaaaacttttgatgtacgattaaataatgtactacaagaagacttccaagagatgtgcagagatatagccagtgacccattaacattcactcagtgggtgggtgctgatgctccttatcaattccttgcatgggcttttgaatatgcaaactacttagatttagttgatgaaggaagagcgcacgagtttaaaactcacttaccagtacatcaggacggctcttgctcaggtattcaacactatagcgctatgttaaaagatgaagtaggtgctcgcgctgttaaccttaagccatcagatgcaccgcaagacatttacggggaagtggcaaaggttgttattcgtaagaataatgagaacatggaagcatcagaagaagatttcatgacttctggctctatgcatctacaaggcgctgtattgcgtgctatggcctcttcatgggatagtgtaggtatcacaaggggattaactaaaaagcccgttatgactcttccctatggttctacgcgtataacttgccgtgagtctgtagaagattatttgattaccttagaagaagaggaagttaaaagagctattgcagaaaaccgcaaggctaacaaggttcacccgttcgttagtgacaaggaagaagggcagattttagagcgtgatgcattaaactacatgacagctttaatatggccttctatctcagaagttgttaaggctcctattgtagcaatgcgtatgataaagcaacttgcaagatatgcatctaaacgcaatgaaggtttagagtacacattaccaactggctttatcttgaaacaaaagattatggctactgagatgctacgtgttcgcactatgttgatgggtgatatccgtatgtccttacaagtagatactgacgtagtagatgaaacagcaatgagtggagcaagtgcaccaaactttgtacacggtcacgatgcaagccacttaatcttgacagtatgtgacttagtagataagggaatcaaatcaatagcagttattcatgattcatttggcacacatgcagataacacagtagctctacgtgactctttacgccataaaatggtggagatgtacgagaacaccaatgtattacagaagttactagaagagcatgaggagagatggctagttgatacaggtatcaaagttccagagcaaggcacttttgatataagagaaatcttaaactcagattatgtattcgcttaatgcacgtgaggatccaactcgagaacttagatggtattagtgacctgtaacagagcattagcgcaaggtgatttttgtcttcttgcgctaattttttgtcatcaaacctgtcgctag

SEQ ID NO: 5 - DNA, 2920 bp, synthetic construct
  misc_feature 1..2920: note = Synthetic: F2278, Screening template for RNApol161
  source 1..2920: mol_type = other DNA
agatctcgatcccgcgaaataattaacccacactatagggctacttccaacaaccacatacaacggtttcactctagaaattgggccactatagcaacaccaaaaccatcatcaggaggaattaaccatgcatcatcatcaccatcacggcagcgctaacgtaatcaaaccagaatctcacaacttctctgatatctctgctgcaatcctaccgttcaacgtattggcagactcttacggtgaggctctggccgctgaacagttgatgcttgagcatgaatcttaccagttaggtgaagcacgattcatcaaagcgatggaacgtcaagtagaacgtggtgaggtgtctgataatgctgtggcgaaaccactgttagacactctaattcctgctctggcagctcgtatcactgagtttgtcgagatgaaacagcgtggtaaacctcacgtatcaaaaggttacttcgctatgattaaacctgagtctgctgcatttatcatcgttaagactactctgaatatccttgctaaagaagagtctgttccggttcaacgtgtagcaatggctatcggtggtaacatcgaagatgagattcgctttggtcgtatccgtgacgaagaaatcaaacacttcaaggagcgcgtaaagcctaacttagacaagcgtaacggtttcatctacaagaaagcatacatggaagctgtggaagctggtatgcaagacaaaggtgagttaaactcaacgcatgaagcttgggaaaaagacgttaagtttcacgttggtatccgtgctattgaaatgctgattgaagcgactggcatggttcaacttgagcgtaagttcaaaggtattccagataaagaccatgaagctttacaccttgcgcctgaatacgtagagaaactgactaaccgtgctcacgctctggcaggaatctcaccgatgtatcaaccaatgattgtcaaacctaagcgttggactggtgtacaaggtggtggctattgggctaaaggtcgtcgtccactgaatctaatccgtgtaggctctaaacgtgctctcgaccgttatcgtcaagtggatatgccagaagtttacgatgcgattaacactattcaggaaaccgcatggcgtattaacaaagatgttcttgctgtagttaacaacgtggttacttgggctaattgtccagtagaagacgttccgtcaatcgacaagcttgctctacctgagaagcctgaagatatcgacagcaacgaggagtcactgaagaaatggaagaaggctgctgctgcaatctatcgtaaagagaaggctcgccaatctcgtcgaatcagcttagagtttgctttgagccaagctaacaagttcagcaagtacaacgagatttacttcccatataacatggactggcgtggtcgcgtatatgcaatcccgatgttcaatccacaaggtaacgatatggtgaaaggcttgctgaccttcgctaagaaagttcctgtaggtatcgacggtggttactggctggctgttcatggtgcgaactgtgcaggcgttgacaaagtatcgcttgaagaccgtgttaaatgggtgaacgataacgaggctaatatccttgcaagtgctgaggctcctttagacttcacttggtgggctgagcaagactctccgttctgcttccttgcattctgcttcgagtgggctgcttatgtgaaagcaggtaagaaaccaagctttgaatcaagcttaccgctggccttcgatggtacttgctctggtctacaacacttctcagcgatgttacgtgatgaaatcggtggagctgctgttaacctgctacctgctgataaaccacaagatatctatggtatcgttgctgtgaaagtgaatgaggtactacgtgaccttgtgattagcggtacagaagacgagatgcaaactctcgaagacaagaagactggtgaaatcactgaacgtcttgtgcttggtactcgcactctggctgctcaatggttagaatatggtgtgactcgctcagtaactaaacgctcagttatgactctggcttatggctctaaagagtatggctttgctgaccaagtgtttgaagatactgtgatgccagctatcgacaatggcaaaggtgcaatgttcactgagccttcacaagcttgtcgtttcatggcgaaactcatttgggatgctgtaagtaaaactgtggtagctgctgtagaagctatgcaatggttacaatcagccgctaaactggtgtcttctgaggtgaaagacaagaagtcaggcgagattctgaaacacgcaatgcctgtgcactggacaacccctaacggtttccctgtatggtcagagtattgcaagcaagaacagaagcgtattgattgcgttatccttggcactcatcgcatggcgttaaccattaacatccgcgataagaaagagattgacgctgcaaaacaaacttcaggtatcgctcctaacttcgttcacagtatggacgcgagtcacctacaaatgacagtcaacaagtgcttcaaagtgtacggtattcactccttcgctatgattcacgatagctttggttgccatgctggttttgcttctaagatgttccgtgcagtacgtgaaacaatggtggaaacttatgaagaacatgatgtaatccaagagttctacaatcagtttgagcaacaacttcacgaatctcaaattgaaaagatgccagttctaccacgcaaaggtaacttggaactgagagaaatcctcaaatctctatacactttctcttaatgcacgtgaggatccaactcgagaacttagatggtattagtgacctgtaacagagcattagcgcaaggtgatttttgtcttcttgcgctaattttttgtcatcaaacctgtcgctag

SEQ ID NO: 6 - DNA, 2896 bp, synthetic construct
  misc_feature 1..2896: note = Synthetic: F2280, Screening template for RNApol136
  source 1..2896: mol_type = other DNA
agatctcgatcccgcgaaataattaggggctactatagaagagagaaacatttacggcgacaacggtttcactctagaaattgggccactatagcaacaccaaaaccatcatcaggaggaattaaccatgcatcatcatcaccatcacggcagccaagatttacacgctattcaactgcaatttgaagaagagatgttcaacggcggcattcgtcgcttcgaggctgacaatcagcgtgttatagccagcggtaacgagtcagaaaccgcatggaatcgccgtttactctctgaattgatcgcacctatggctgaaggcatcaacgcctataaagaatcttacgttggcaagcgtggaaaacctgctttagcccttgccttccttcagtgcgtagagaatgaagttgcagcatatatcacaatgaaggttgttatggatatgctcaacactgacgtaaccctgcaagccgttgcgatgaccattgctgagcgtattgaagatcaggtacgcttctctaagctggatgcacacgccaacaaatactttgagaaggttaaggccagcctcaaggcgtcaaaatctaagcaataccgccacgggcaccgcgtaatggtagccgctgagaaatcagttagtgagaaagatgccgattttgaccgctgggaagcatggcctaaagaaacttgcttgcaaatcggggctaccttgctggatattctggaatctagtgtgttctatcagggtgagcctgtattcttccgcgctatccgtcaaaatggcgtgcgctctacgtactaccttcagacttctgagactgtgggggcatggattgaggagtttaaagagcacgtggcacagcttgccccagcttatgccccttgtgttgtccctccccgtgactggaaaagcccgttcaatggtggattccatactgagaaggtatcaagccgtgtgcgtctggttaaaggtgcgcgtgagcatgtacgcaagttgaccgttaagcagatgccgaatgtgtacaaggccatcaacgcattgcagcgcactgaatggcaagttaacactgacgttatgaaagtcgctgatgacgttatccgccttaacctaggctatgggatgccgtcctttaagcctcttatcgataaagagaacaagccacttaaccctgtgcctgtggagtttcagcatttgcgcgggcgtgagttgaaagaaatgctcaccgctgagcaatgggatacgtttatcgcatggaaaggtgagtgctcacgcctgtataccgctgaaactaagcgaggctctaagagcgccgccgtggttcgcatggtgggacaagcccgtaaatactccgcgtttaacgccatccattttgtttacgcgatggattcccgcagccgtgtatatgcgcaatcctccacgctgtcaccgcaatccaatgacttaggcaaggcattgctccgctttactgagaaacgcgccctaaatggctctcaggcgctcaaatggttctgtgtcgcgggcgctaacctttggggctgggacaagaaaaccttcggcgtgcgcgtgtccaacgtgttagacggcgagtttcaagatatgtgtcgtgatatcgccgctgacccgctcacctttacacagtgggctggcgcggacgagccttatcagttcctagcgtgggcgatggaatacgcgaactaccttgacctgttagatgaagaccgtcaagaagagttccgtacacagttgccagttcatcaggacggctcttgctcaggcatccagcactattccgcaatgctgaaggacagcgtaggcgcggcggcggtcaacctcctgcctagcgacgaaccacaggatatctacgggcgggtagctcaggtagttatcggcaaggtggcacagcacgctaacgcactggacgcggatacgttcacttctgggaagctgacactcacaggcgatgcactgcgcaccatggcggcatcgtgggacgcaataggtatcacgcgcggtctgactaaaaagccagtcatgacactgccgtatggctcaacccgtatcacctgccgtgaagctgtagccgattacctgattgacctagaagagaaagaggctcaaaaggcggtggcagaagggcgaggagtgaaccttgtgcacccattcggacaggcagaaggacagatgaccgaagcgatggccttgaactacatgacggcgcttatctggccttccatttctgaagttgttaaggctccaatcgtcgcaatgaagatgatccgtgcgctggctcgctttgctgcgaaacgaaatgaagggctggagtatacgctccctactggcttcattcttcagcaaaagattatggcgaccgaccttctgcgcgtgcgcactgtgttgagcggcgatataaaaatggctctgcgactgcaagtcgataccgatattgtagacgaatcagcgatgatgggtgctgcggctcctaactttgttcacggacacgatgcgagccacttgattctgtccgtctgcgcaatggtggacgaagggattacctcaattgcagtcattcacgactctttcggtactcacgcagaccacacaccagacctccgcaacgcactgaaagggcagatggttgagatgtacgccaacactaacgcgctccagaaactgctgagcgaacacgaagaccgctggatggtagatactaagattgaagtaccggagcaaggtgactttgacgtaaacctgattatggagtcggaatactgcttcgcataatgcacgtgaggatccaactcgagaacttagatggtattagtgacctgtaacagagcattagcgcaaggtgatttttgtcttcttgcgctaattttttgtcatcaaacctgtcgctag

SEQ ID NO: 7 - DNA, 40 bp, synthetic construct
  misc_feature 1..40: note = Synthetic: PG5004, Mutagensis primer (Also RNApol137, insert #2, reverse primer)
  source 1..40: mol_type = other DNA
gagttggatcctcacgtgcatcacgcaaaagcgtatgtag

SEQ ID NO: 8 - DNA, 27 bp, synthetic construct
  misc_feature 1..27: note = Synthetic: PG5826, Mutagensis primer (and forward primer for multiple inserts later)
  source 1..27: mol_type = other DNA
atgcatcatcatcaccatcacggcagc

SEQ ID NO: 9 - DNA, 34 bp, synthetic construct
  misc_feature 1..34: note = Synthetic: PG0599, Fragment #3 amplifiation (Many applications) & universal template regen
  source 1..34: mol_type = other DNA
tgcacgtgaggatccaactcgagaacttagatgg

SEQ ID NO: 10 - DNA, 24 bp, synthetic construct
  misc_feature 1..24: note = Synthetic: PG5436PT, Fragment #3 amplifiation, many other applications
  modified_base 1..4: mod_base = OTHER; note = phosphorothioate linkages
  source 1..24: mol_type = other DNA
ctagcgacaggtttgatgacaaaa

SEQ ID NO: 11 - DNA, 30 bp, synthetic construct
  misc_feature 1..30: note = Synthetic: PG5876, Primer for cloning (RNApol137) after assembly
  source 1..30: mol_type = other DNA
agatctcgatcccgcgaaattaggcactac

SEQ ID NO: 12 - DNA, 24 bp, synthetic construct
  misc_feature 1..24: note = Synthetic: PG5916, qPCR Target forward primer (for templates with RNApol031 initiator)
  source 1..24: mol_type = other DNA
caacggtttcactctagaaattgg

SEQ ID NO: 13 - DNA, 19 bp, synthetic construct
  misc_feature 1..19: note = Synthetic: PG5917, qPCR target reverse primer
  source 1..19: mol_type = other DNA
ctgccgtgatggtgatgat

SEQ ID NO: 14 - DNA, 21 bp, synthetic construct
  misc_feature 1..21: note = Synthetic: PG5776, qPCR forward primer (total transcripts) - RNApol137
  source 1..21: mol_type = other DNA
cctgttatgaccctgccttac

SEQ ID NO: 15 - DNA, 21 bp, synthetic construct
  misc_feature 1..21: note = Synthetic: PG5777, qPCR reverse primer (total transcripts) - RNApol137
  source 1..21: mol_type = other DNA
cttagctcgcttgttctcctc

SEQ ID NO: 16 - DNA, 74 bp, synthetic construct
  misc_feature 1..74: note = Synthetic: PG6177, Selective target amplification from cDNA and template regeneration
  modified_base 69..72: mod_base = OTHER; note = phosphorothioate linkages
  source 1..74: mol_type = other DNA
agatctcgatcccgcgaaattaggcactacaataggagaagctgaaagtaacaacggtttcactctagaaattg

SEQ ID NO: 17 - DNA, 24 bp, synthetic construct
  misc_feature 1..24: note = Synthetic: PG5909, Selective target amplification from cDNA and template regeneration
  source 1..24: mol_type = other DNA
ccatctaagttctcgagttggatc

SEQ ID NO: 18 - DNA, 6268 bp, synthetic construct
  misc_feature 1..6268: note = Synthetic: PP0396, Source of terminator
  source 1..6268: mol_type = other DNA
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 19 - DNA, 7369 bp, synthetic construct
  misc_feature 1..7369: note = Synthetic: PP1243, Source of reporter construct
  source 1..7369: mol_type = other DNA
agcggaagaggcgccagcaaccgcacctgtggcgccggtgatgccggccacgatgcgtccggcgtagaggatcgagatctcgatcccgcgaaattaatacgactcactatagggagaccacaacccacaacaaaccccagctgttggggccaggacacccagtgagcccatacttgctctttttgtcttcttcagactgcgccatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaaggcagcggatccgaggatgccaagaacatcaagaaaggcccagccccattttaccccttggaggatggcactgctggtgaacagctccacaaagcgatgaagcgctacgccttggtgccgggtaccattgcgtttacagacgcccacatcgaggtgaacataacctatgccgagtacttcgaaatgtcggttcggttggcggaggcaatgaagaggtatggcctgaacaccaaccaccgcattgtcgtgtgttcggagaattccctgcaattctttatgcctgtgcttggtgccttgttcatcggcgttgccgttgcacccgctaatgacatatacaacgagagggagctcctgaactcgatgaacatatcccaaccgacggttgtctttgtctccaagaagggacttcagaagatcctgaatgtgcagaagaagctgccaatcattcagaagatcatcatcatggactctaagaccgactaccagggcttccaatccatgtacaccttcgtgacctcacacttgccacccggattcaacgagtacgacttcgttcctgagtccttcgaccgcgataagaccatagcgctgatcatgaatagctctggcagcaccggactgccaaagggtgtggctcttccgcatagaacagcgtgcgttcgtttcagtcatgcacgggacccgattttcgggaaccagatcatacccgatacggccatcctctcagtggtcccatttcaccacgggttcggcatgtttaccactctggggtacctgatctgcgggtttcgcgtggtcctgatgtaccgttttgaggaggaactgttcctgaggagccttcaggactacaagatccaaagtgccttgctcgtgccaacactcttcagtttcttcgcgaaatccacgctgatcgacaagtatgacctgtcaaacctgcacgagatcgcctctggaggggctcctctcagcaaagaggtgggcgaagcagtggccaaacgctttcatctgcccggaatcaggcaagggtatggcctcacggagactaccagcgcgattctcatcacaccggaaggagatgacaagcctggcgccgtcggtaaagtcgtgcccttcttcgaagcaaaggtggtggatctcgataccggcaagaccctcggcgtcaaccagagaggggaactgtgcgtcagaggtcctatgatcatgagcggatacgtgaacaatcccgaggccactaatgccctgatcgacaaagatgggtggcttcattccggcgacattgcctactgggacgaggacgagcacttcttcatagttgaccggctgaagtctctgatcaagtacaaaggctatcaggtcgctcctgccgaactggagagcattctcctgcagcaccccaacatcttcgatgcaggtgtggcggggcttccggacgacgatgctggagaactgccagcagctgtggtcgttctcgaacatgggaaaacgatgacggagaaagagatcgtggactatgtggccagccaagtgactacagcgaagaagctgaggggcggagtcgtctttgtggacgaggtgcccaaagggctgacaggcaagctcgatgctcgcaagattcgggaaattctcattaaggccaagaaaggcggcaagtcaaagctgtgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatccaacacgtgagtatcaagcggccgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagga

SEQ ID NO: 20 - DNA, 77 bp, synthetic construct
  misc_feature 1..77: note = Synthetic: PG6787, Reporter construct forward primer
  source 1..77: mol_type = other DNA
atcccgcgaaattaggcactacaataggagaagctgaaagtaactatatattacggattcactggccgtcgttttac

SEQ ID NO: 21 - DNA, 113 bp, synthetic construct
  misc_feature 1..113: note = Synthetic: PG6740, Reporter construct reverse primer
  source 1..113: mol_type = other DNA
ctagcgacaggtttgatgacaaaaaattagcgcaagaagacaaaaatcaccttgcgctaatgctctgttacaggtcactaatacctcacagctttgacttgccgcctttcttg

SEQ ID NO: 22 - DNA, 24 bp, synthetic construct
  misc_feature 1..24: note = Synthetic: PG0338D, RT primer
  source 1..24: mol_type = other DNA
gctctgttacaggtcactaatacc

SEQ ID NO: 23 - DNA, 2890 bp, synthetic construct
  misc_feature 1..2890: note = Synthetic: F3631, Screening template for Prima III (RNApol157) - AT TSS
  source 1..2890: mol_type = other DNA
agatctcgatcccgcgaaatttgattaattaacccacactataatgagagaggggcacaacggtttcactctagaaaattaggggctactatagaagagagaaacatttacggcgaaggaggaaaaaatatgcatcatcatcaccatcacggcagcgctaacgtaatcaaaccagaatctcacaacttctcagatatctctgctgcaatcctaccgttcaacgtacttgcagactcttacggtgaggctctggccgctgaacagttgatgcttgagcatgaatcttaccagttaggtgaagcacgattcatcaaagcgatggaacgtcaagtagaacgtggtgaggtgtctgataatgctgttgctaaaccactgttagacactctaattcctgctctggcagctcgtatcactgagtttatagagatgaaacagcgcggtaaacctcacgtatcaaaaggttacttcgctatgattaagcctgagtctgctgcttttatcatcgttaagactactctgaatatccttgctaaagaagagtctgttccggttcaacgtgtagcaatggctatcggtggtaacatcgaagatgagattcgctttggtcgtatccgtgatgaagaaatcaaacacttcaaggagcgcgttaagcctaacttagacaagcgcaacgggtttatctacaagaaagcatacatggaagctgttgaagctggtatgcaagataaaggcgagttaaactctacgcatgaagcttgggaaaaagacgttaagtttcacgttggtatccgtgctattgaaatgcttatcgaagctactggcatggttcaacttgagcgtaagttcaaaggtatcccagataaagaccatgaagctttacaccttgcgcctgaatacgtagagaaactgactaaccgtgctcacgctctggcaggtatctcaccgatgtatcaaccaatgattgtcaaacctaagcgttggactggtgtacaaggtggtggctattgggctaaaggtcgtcgtccactgaacctaatccgtgtaggctctaagcgtgctctcgaccgttatcgtcaagtggatatgcctgaagtttacgatgcgattaacactattcaggagaccgcatggcgcattaacaaagatgttcttgctgtagttaacaacgtggtcacttgggctaactgtccggtagaagacgttccgtcaatcgacaagcttgctctacctgagaagcctgaagatatcgacaacaacgaggagtcactgaagaaatggaagaaggctgctgctgcaatctatcgtaaagagaaggctcgccaatctcgtcgaatcagcttagagtttgctttgagtcaagctaataagttcagcaagtacaacgagatttacttcccatacaacatggactggcgtggtcgcgtgtatgcaatcccgatgttcaatccacaaggtaacgatatggtgaaaggcttactgaccttcgctaagaaagttcctgtaggtatcgacggtggttactggttggctgttcatggtgcgaactgtgcaggcgttgacaaagtatcgcttgaagaccgtgttaaatgggtgaacgataacgaagctaatatcattgcaagcgctgaggctcctttagacttcacttggtgggctgagcaagactctcctttctgcttccttgcgttctgcttcgagtgggctgcgtatgtgaaagcaggtaagaaaccaagctttgagtcaagcttaccgctggccttcgatggtacttgctctggtctacaacacttctcagcgatgttacgtgatgaaatcggtggagctgctgttaacctgctacctgctgataaaccacaagatatctatggtatcgttgctgtgaaagtgaatgaggtactacgtgaccttgtgattagcggtacagaagacgagatgcaaactctcgaagacaagaagactggtgaaatcactgaacgtcttgtgcttggtactcgcactctggctgctcaatggttagaacatggtgtgactcgctcagtaactaaacgctcagttatgactctggcttatggttctaaggagtatggcttcgctgaccaagtgtttgaggacactgtgatgccagctatcgacaacggcaaaggtgctatgttcactgagccttcacaagcttgtcgtttcatggcgaaactcatttgggatgctgtaagtaaaactgtggttgctgctgtagaagctatgcaatggttacaatcagccgctaaactggtgtcttctgaggtgaaagacaagaagtcaggtgagattctgaaacatgcaatgcctgtacactggacgactcctaacggtttccctgtatggtctgagtattgcaaacaagaacagaagcgtattgattgcgttatcctcggtactcatcgcatggcgttaaccattaacatccgcgataagaaagagattgacgctgcaaaacagacttcaggtatcgctcctaacttcgttcacagtatggatgcgagtcacctacaaatgacagtcaacaagtgcttcaaagtgtatggtattcactccttcgctatgattcacgacagttttggttgccatgctggttttgcttctaagatgttccgtgcagtacgtgaaacaatggtagagacttatgaagaacatgatgtaatccaagagttctacaatcagtttgagcaacaacttcacgaatctcaaattgaaaagataccagttctaccacgcaaaggtaacttggagctgagagaaatcctcaagtctctatacactttctcttaaggtattagtgacctgtaacagagcattagcgcaaggtgatttttgtcttcttgcgctaattttttgtcatcaaacctgtcgctag

SEQ ID NO: 24 - DNA, 2890 bp, synthetic construct
  misc_feature 1..2890: note = Synthetic: F3626, Screening template for Prima III (RNAP157) - AG TSS
  source 1..2890: mol_type = other DNA
agatctcgatcccgcgaaatttgattaattaacccacactataaggagagaggggcacaacggtttcactctagaaaattaggggctactatagaagagagaaacatttacggcgaaggaggaaaaaatatgcatcatcatcaccatcacggcagcgctaacgtaatcaaaccagaatctcacaacttctcagatatctctgctgcaatcctaccgttcaacgtacttgcagactcttacggtgaggctctggccgctgaacagttgatgcttgagcatgaatcttaccagttaggtgaagcacgattcatcaaagcgatggaacgtcaagtagaacgtggtgaggtgtctgataatgctgttgctaaaccactgttagacactctaattcctgctctggcagctcgtatcactgagtttatagagatgaaacagcgcggtaaacctcacgtatcaaaaggttacttcgctatgattaagcctgagtctgctgcttttatcatcgttaagactactctgaatatccttgctaaagaagagtctgttccggttcaacgtgtagcaatggctatcggtggtaacatcgaagatgagattcgctttggtcgtatccgtgatgaagaaatcaaacacttcaaggagcgcgttaagcctaacttagacaagcgcaacgggtttatctacaagaaagcatacatggaagctgttgaagctggtatgcaagataaaggcgagttaaactctacgcatgaagcttgggaaaaagacgttaagtttcacgttggtatccgtgctattgaaatgcttatcgaagctactggcatggttcaacttgagcgtaagttcaaaggtatcccagataaagaccatgaagctttacaccttgcgcctgaatacgtagagaaactgactaaccgtgctcacgctctggcaggtatctcaccgatgtatcaaccaatgattgtcaaacctaagcgttggactggtgtacaaggtggtggctattgggctaaaggtcgtcgtccactgaacctaatccgtgtaggctctaagcgtgctctcgaccgttatcgtcaagtggatatgcctgaagtttacgatgcgattaacactattcaggagaccgcatggcgcattaacaaagatgttcttgctgtagttaacaacgtggtcacttgggctaactgtccggtagaagacgttccgtcaatcgacaagcttgctctacctgagaagcctgaagatatcgacaacaacgaggagtcactgaagaaatggaagaaggctgctgctgcaatctatcgtaaagagaaggctcgccaatctcgtcgaatcagcttagagtttgctttgagtcaagctaataagttcagcaagtacaacgagatttacttcccatacaacatggactggcgtggtcgcgtgtatgcaatcccgatgttcaatccacaaggtaacgatatggtgaaaggcttactgaccttcgctaagaaagttcctgtaggtatcgacggtggttactggttggctgttcatggtgcgaactgtgcaggcgttgacaaagtatcgcttgaagaccgtgttaaatgggtgaacgataacgaagctaatatcattgcaagcgctgaggctcctttagacttcacttggtgggctgagcaagactctcctttctgcttccttgcgttctgcttcgagtgggctgcgtatgtgaaagcaggtaagaaaccaagctttgagtcaagcttaccgctggccttcgatggtacttgctctggtctacaacacttctcagcgatgttacgtgatgaaatcggtggagctgctgttaacctgctacctgctgataaaccacaagatatctatggtatcgttgctgtgaaagtgaatgaggtactacgtgaccttgtgattagcggtacagaagacgagatgcaaactctcgaagacaagaagactggtgaaatcactgaacgtcttgtgcttggtactcgcactctggctgctcaatggttagaacatggtgtgactcgctcagtaactaaacgctcagttatgactctggcttatggttctaaggagtatggcttcgctgaccaagtgtttgaggacactgtgatgccagctatcgacaacggcaaaggtgctatgttcactgagccttcacaagcttgtcgtttcatggcgaaactcatttgggatgctgtaagtaaaactgtggttgctgctgtagaagctatgcaatggttacaatcagccgctaaactggtgtcttctgaggtgaaagacaagaagtcaggtgagattctgaaacatgcaatgcctgtacactggacgactcctaacggtttccctgtatggtctgagtattgcaaacaagaacagaagcgtattgattgcgttatcctcggtactcatcgcatggcgttaaccattaacatccgcgataagaaagagattgacgctgcaaaacagacttcaggtatcgctcctaacttcgttcacagtatggatgcgagtcacctacaaatgacagtcaacaagtgcttcaaagtgtatggtattcactccttcgctatgattcacgacagttttggttgccatgctggttttgcttctaagatgttccgtgcagtacgtgaaacaatggtagagacttatgaagaacatgatgtaatccaagagttctacaatcagtttgagcaacaacttcacgaatctcaaattgaaaagataccagttctaccacgcaaaggtaacttggagctgagagaaatcctcaagtctctatacactttctcttaaggtattagtgacctgtaacagagcattagcgcaaggtgatttttgtcttcttgcgctaattttttgtcatcaaacctgtcgctag

SEQ ID NO: 25 - DNA, 2867 bp, synthetic construct
  misc_feature 1..2867: note = Synthetic: F3638, Screening template for RNApol126 - AT TSS
  source 1..2867: mol_type = other DNA
agatctcgatcccgcgaaattggtcttaattagagaccactataatagagataaattacaacggtttcactctagaaaattaggggctactatagaagagagaaacatttacggcgaaggaggaaaaaatatgcatcatcatcaccatcacggcagcgatttacaagaaattcaattacagttagagaatgaaatgtttaacggtggcattcgtagattcgaagcagaccaacaaagacatttacaggcaggtaatgcttctgatactgcatggaatcgtaagcttatctctgagtttatcgcacctatggcacaaggtattcaggcatataaagaagagtacaaaggcaagataggtcgtgctcctcgtgctttagctttcctaaattgtgtagaaaatgaagtgtctgcatatattactatgaaggtagtaatggatatgatacagacagacgtcactttacaatcaatagctatgagtatagctgaccgtattgaagaccaagttagattttctaacttagaaggcaaagctaagaaatactttgagaaagtaaaagcaagtcttaaagcttcacgctctaaacaatatgcacatggtcataaagttatggtagtagctgaaaagaatcttgctgagaagagcgaagatatagaccgttggataccttggggtaaagaggaattactaaatattggtcttactttacttgagatattagaaaactctgtgttctttaatggtgaacctgtcttcttccgtactattcgcactatgggttataagaaaacattatatttcttacagacatctgagaatattggtgaatgggtacgtgcatttaaagaccatgtagcgcaactaagccctgcatatgcaccttgtgttgttcctcctcgtccttggaaatctccttttaatggtggcttccatactgagaaagtagctagtcgcattcgtcttgttaaaggtgaccgcgagcatgtacgtaagttaacacaaaagcagatgccgaaagtgtacaaagctattaatgctttacaaaatacacaatggcaagttaacaaagatattctaaacgtagctgatgaggttgtaagattagaccttggttatggtgtgccacacttcaaacctttaattgataaagataacaagccagctaatcctgtgcctgttgaattccagcacctaagaggtagagagttaaaagaagtgttaagtgatgagcaatggcaagcctttatccactggaaaggtgaatgtgccaagctatacacagcagaaactaagagaggttctaaatctgcttcggtggttagaatgttaggacaagcgcgcaaatatagcatgtttgatgcaatatactttgtatatgctatggattcacgttctcgcgtatacgctcagagtagcacgctaagcccacaaagcgacgacttaggtaaatcattattgagattcacagaagggcgtagcattaattctgtggaagatttgaagtggttctgcattaatggtgctaacctttggggttgggataagaaaacttttgatgtacgattaaataatgtactacaagaagacttccaagagatgtgcagagatatagccagtgacccattaacattcactcagtgggtgggtgctgatgctccttatcaattccttgcatgggcttttgaatatgcaaactacttagatttagttgatgaaggaagagcgcacgagtttaaaactcacttaccagtacatcaggacggctcttgctcaggtattcaacactatagcgctatgttaaaagatgaagtaggtgctcgcgctgttaaccttaagccatcagatgcaccgcaagacatttacggggaagtggcaaaggttgttattcgtaagaataatgagaacatggaagcatcagaagaagatttcatgacttctggctctatgcatctacaaggcgctgtattgcgtgctatggcctcttcatgggatagtgtaggtatcacaaggggattaactaaaaagcccgttatgactcttccctatggttctacgcgtataacttgccgtgagtctgtagaagattatttgattaccttagaagaagaggaagttaaaagagctattgcagaaaaccgcaaggctaacaaggttcacccgttcgttagtgacaaggaagaagggcagattttagagcgtgatgcattaaactacatgacagctttaatatggccttctatctcagaagttgttaaggctcctattgtagcaatgcgtatgataaagcaacttgcaagatatgcatctaaacgcaatgaaggtttagagtacacattaccaactggctttatcttgaaacaaaagattatggctactgagatgctacgtgttcgcactatgttgatgggtgatatccgtatgtccttacaagtagatactgacgtagtagatgaaacagcaatgagtggagcaagtgcaccaaactttgtacacggtcacgatgcaagccacttaatcttgacagtatgtgacttagtagataagggaatcaaatcaatagcagttattcatgattcatttggcacacatgcagataacacagtagctctacgtgactctttacgccataaaatggtggagatgtacgagaacaccaatgtattacagaagttactagaagagcatgaggagagatggctagttgatacaggtatcaaagttccagagcaaggcacttttgatataagagaaatcttaaactcagattatgtattcgcttaaggtattagtgacctgtaacagagcattagcgcaaggtgatttttgtcttcttgcgctaattttttgtcatcaaacctgtcgctag

SEQ ID NO: 26 - DNA, 90 bp, synthetic construct
  misc_feature 1..90: note = Synthetic: PG8520, Regenerative primer for RNApol157 - AG TSS
  source 1..90: mol_type = other DNA
agatctcgatcccgcgaaatttgattaattaacccacactataaggagagaggggcacaacggtttcactctagaaaattaggggctact

SEQ ID NO: 27 - DNA, 90 bp, synthetic construct
  misc_feature 1..90: note = Synthetic: PG8523, Regenerative primer for RNApol126 - AT TSS
  source 1..90: mol_type = other DNA
agatctcgatcccgcgaaattggtcttaattagagaccactataatagagataaattacaacggtttcactctagaaaattaggggctac

SEQ ID NO: 28 - DNA, 90 bp, synthetic construct
  misc_feature 1..90: note = Synthetic: PG8525, Regenerative primer for RNApol157 - AT TSS
  source 1..90: mol_type = other DNA
agatctcgatcccgcgaaatttgattaattaacccacactataatgagagaggggcacaacggtttcactctagaaaattaggggctact

SEQ ID NO: 29 - DNA, 32 bp, synthetic construct
  misc_feature 1..32: note = Synthetic: PG8831, Regenerative reverse primer (R0 and R1)
  source 1..32: mol_type = other DNA
gcgctaatgctctgttacaggtcactaatacc

SEQ ID NO: 30 - DNA, 86 bp, synthetic construct
  misc_feature 1..86: note = Synthetic: PG6684, Mutazyme forward primer (RNApol136 initiator)
  source 1..86: mol_type = other DNA
cactctagaaaattaggggctactatagaagagagaaacatttacggcgaaggaggaaaaaatatgcatcatcatcaccatcacgg

SEQ ID NO: 31 - DNA, 60 bp, synthetic construct
  misc_feature 1..60: note = Synthetic: PG8826, RNApol126 mutazyme reverse primer
  source 1..60: mol_type = other DNA
gcgctaatgctctgttacaggtcactaataccttaagcgaatacataatctgagtttaag

SEQ ID NO: 32 - DNA, 60 bp, synthetic construct
  misc_feature 1..60: note = Synthetic: PG8828, RNApol157 mutazyme reverse primer
  source 1..60: mol_type = other DNA
gcgctaatgctctgttacaggtcactaataccttaagagaaagtgtatagagacttgagg

SEQ ID NO: 33 - DNA, 22 bp, synthetic construct
  misc_feature 1..22: note = Synthetic: PG7322, qPCR Universal Target forward primer
  source 1..22: mol_type = other DNA
cggtttcactctagaaaattag

SEQ ID NO: 34 - DNA, 19 bp, synthetic construct
  misc_feature 1..19: note = Synthetic: PG2146, qPCR Universal all-transcript forward primer
  source 1..19: mol_type = other DNA
tcatcaccatcacggcagc

SEQ ID NO: 35 - DNA, 21 bp, synthetic construct
  misc_feature 1..21: note = Synthetic: PG5987, qPCR all-transcript reverse primer for RNApol126 templates
  source 1..21: mol_type = other DNA
tctacgaatgccaccgttaaa

SEQ ID NO: 36 - DNA, 21 bp, synthetic construct
  misc_feature 1..21: note = Synthetic: PG6376, qPCR all-transcript reverse primer for RNApol157 templates
  source 1..21: mol_type = other DNA
ctcaagcatcaactgttcagc

SEQ ID NO: 37 - DNA, 85 bp, synthetic construct
  misc_feature 1..85: note = Synthetic: PG6776, reverse primer for template regeneration from cDNA (R2 and on)
  source 1..85: mol_type = other DNA
ctagcgacaggtttgatgacaaaaaattagcgcaagaagacaaaaatcaccttgcgctaatgctctgttacaggtcactaatacc

SEQ ID NO: 38 - DNA, 20 bp, synthetic construct
  misc_feature 1..20: note = Synthetic: PG6169, forward primer for re-amplifying full-length template (universal)
  modified_base 2..5: mod_base = OTHER; note = phosphorothioate linkages
  source 1..20: mol_type = other DNA
agatctcgatcccgcgaaat

SEQ ID NO: 39 - DNA, 2837 bp, synthetic construct
  misc_feature 1..2837: note = Synthetic: F3672, RNAP157 Screening template (no terminator) - AG TSS
  source 1..2837: mol_type = other DNA
agatctcgatcccgcgaaatttgattaattaacccacactataaggagagaggggcacaacggtttcactctagaaaattaggggctactatagaagagagaaacatttacggcgaaggaggaaaaaatatgcatcatcatcaccatcacggcagcgctaacgtaatcaaaccagaatctcacaacttctcagatatctctgctgcaatcctaccgttcaacgtacttgcagactcttacggtgaggctctggccgctgaacagttgatgcttgagcatgaatcttaccagttaggtgaagcacgattcatcaaagcgatggaacgtcaagtagaacgtggtgaggtgtctgataatgctgttgctaaaccactgttagacactctaattcctgctctggcagctcgtatcactgagtttatagagatgaaacagcgcggtaaacctcacgtatcaaaaggttacttcgctatgattaagcctgagtctgctgcttttatcatcgttaagactactctgaatatccttgctaaagaagagtctgttccggttcaacgtgtagcaatggctatcggtggtaacatcgaagatgagattcgctttggtcgtatccgtgatgaagaaatcaaacacttcaaggagcgcgttaagcctaacttagacaagcgcaacgggtttatctacaagaaagcatacatggaagctgttgaagctggtatgcaagataaaggcgagttaaactctacgcatgaagcttgggaaaaagacgttaagtttcacgttggtatccgtgctattgaaatgcttatcgaagctactggcatggttcaacttgagcgtaagttcaaaggtatcccagataaagaccatgaagctttacaccttgcgcctgaatacgtagagaaactgactaaccgtgctcacgctctggcaggtatctcaccgatgtatcaaccaatgattgtcaaacctaagcgttggactggtgtacaaggtggtggctattgggctaaaggtcgtcgtccactgaacctaatccgtgtaggctctaagcgtgctctcgaccgttatcgtcaagtggatatgcctgaagtttacgatgcgattaacactattcaggagaccgcatggcgcattaacaaagatgttcttgctgtagttaacaacgtggtcacttgggctaactgtccggtagaagacgttccgtcaatcgacaagcttgctctacctgagaagcctgaagatatcgacaacaacgaggagtcactgaagaaatggaagaaggctgctgctgcaatctatcgtaaagagaaggctcgccaatctcgtcgaatcagcttagagtttgctttgagtcaagctaataagttcagcaagtacaacgagatttacttcccatacaacatggactggcgtggtcgcgtgtatgcaatcccgatgttcaatccacaaggtaacgatatggtgaaaggcttactgaccttcgctaagaaagttcctgtaggtatcgacggtggttactggttggctgttcatggtgcgaactgtgcaggcgttgacaaagtatcgcttgaagaccgtgttaaatgggtgaacgataacgaagctaatatcattgcaagcgctgaggctcctttagacttcacttggtgggctgagcaagactctcctttctgcttccttgcgttctgcttcgagtgggctgcgtatgtgaaagcaggtaagaaaccaagctttgagtcaagcttaccgctggccttcgatggtacttgctctggtctacaacacttctcagcgatgttacgtgatgaaatcggtggagctgctgttaacctgctacctgctgataaaccacaagatatctatggtatcgttgctgtgaaagtgaatgaggtactacgtgaccttgtgattagcggtacagaagacgagatgcaaactctcgaagacaagaagactggtgaaatcactgaacgtcttgtgcttggtactcgcactctggctgctcaatggttagaacatggtgtgactcgctcagtaactaaacgctcagttatgactctggcttatggttctaaggagtatggcttcgctgaccaagtgtttgaggacactgtgatgccagctatcgacaacggcaaaggtgctatgttcactgagccttcacaagcttgtcgtttcatggcgaaactcatttgggatgctgtaagtaaaactgtggttgctgctgtagaagctatgcaatggttacaatcagccgctaaactggtgtcttctgaggtgaaagacaagaagtcaggtgagattctgaaacatgcaatgcctgtacactggacgactcctaacggtttccctgtatggtctgagtattgcaaacaagaacagaagcgtattgattgcgttatcctcggtactcatcgcatggcgttaaccattaacatccgcgataagaaagagattgacgctgcaaaacagacttcaggtatcgctcctaacttcgttcacagtatggatgcgagtcacctacaaatgacagtcaacaagtgcttcaaagtgtatggtattcactccttcgctatgattcacgacagttttggttgccatgctggttttgcttctaagatgttccgtgcagtacgtgaaacaatggtagagacttatgaagaacatgatgtaatccaagagttctacaatcagtttgagcaacaacttcacgaatctcaaattgaaaagataccagttctaccacgcaaaggtaacttggagctgagagaaatcctcaagtctctatacactttctcttaaggtattagtgacctgtaacagagcattagcgc

SEQ ID NO: 40 - DNA, 2814 bp, synthetic construct
  misc_feature 1..2814: note = Synthetic: F3674, RNApol126 Screening template (no terminator) - AT TSS
  source 1..2814: mol_type = other DNA
agatctcgatcccgcgaaattggtcttaattagagaccactataatagagataaattacaacggtttcactctagaaaattaggggctactatagaagagagaaacatttacggcgaaggaggaaaaaatatgcatcatcatcaccatcacggcagcgatttacaagaaattcaattacagttagagaatgaaatgtttaacggtggcattcgtagattcgaagcagaccaacaaagacatttacaggcaggtaatgcttctgatactgcatggaatcgtaagcttatctctgagtttatcgcacctatggcacaaggtattcaggcatataaagaagagtacaaaggcaagataggtcgtgctcctcgtgctttagctttcctaaattgtgtagaaaatgaagtgtctgcatatattactatgaaggtagtaatggatatgatacagacagacgtcactttacaatcaatagctatgagtatagctgaccgtattgaagaccaagttagattttctaacttagaaggcaaagctaagaaatactttgagaaagtaaaagcaagtcttaaagcttcacgctctaaacaatatgcacatggtcataaagttatggtagtagctgaaaagaatcttgctgagaagagcgaagatatagaccgttggataccttggggtaaagaggaattactaaatattggtcttactttacttgagatattagaaaactctgtgttctttaatggtgaacctgtcttcttccgtactattcgcactatgggttataagaaaacattatatttcttacagacatctgagaatattggtgaatgggtacgtgcatttaaagaccatgtagcgcaactaagccctgcatatgcaccttgtgttgttcctcctcgtccttggaaatctccttttaatggtggcttccatactgagaaagtagctagtcgcattcgtcttgttaaaggtgaccgcgagcatgtacgtaagttaacacaaaagcagatgccgaaagtgtacaaagctattaatgctttacaaaatacacaatggcaagttaacaaagatattctaaacgtagctgatgaggttgtaagattagaccttggttatggtgtgccacacttcaaacctttaattgataaagataacaagccagctaatcctgtgcctgttgaattccagcacctaagaggtagagagttaaaagaagtgttaagtgatgagcaatggcaagcctttatccactggaaaggtgaatgtgccaagctatacacagcagaaactaagagaggttctaaatctgcttcggtggttagaatgttaggacaagcgcgcaaatatagcatgtttgatgcaatatactttgtatatgctatggattcacgttctcgcgtatacgctcagagtagcacgctaagcccacaaagcgacgacttaggtaaatcattattgagattcacagaagggcgtagcattaattctgtggaagatttgaagtggttctgcattaatggtgctaacctttggggttgggataagaaaacttttgatgtacgattaaataatgtactacaagaagacttccaagagatgtgcagagatatagccagtgacccattaacattcactcagtgggtgggtgctgatgctccttatcaattccttgcatgggcttttgaatatgcaaactacttagatttagttgatgaaggaagagcgcacgagtttaaaactcacttaccagtacatcaggacggctcttgctcaggtattcaacactatagcgctatgttaaaagatgaagtaggtgctcgcgctgttaaccttaagccatcagatgcaccgcaagacatttacggggaagtggcaaaggttgttattcgtaagaataatgagaacatggaagcatcagaagaagatttcatgacttctggctctatgcatctacaaggcgctgtattgcgtgctatggcctcttcatgggatagtgtaggtatcacaaggggattaactaaaaagcccgttatgactcttccctatggttctacgcgtataacttgccgtgagtctgtagaagattatttgattaccttagaagaagaggaagttaaaagagctattgcagaaaaccgcaaggctaacaaggttcacccgttcgttagtgacaaggaagaagggcagattttagagcgtgatgcattaaactacatgacagctttaatatggccttctatctcagaagttgttaaggctcctattgtagcaatgcgtatgataaagcaacttgcaagatatgcatctaaacgcaatgaaggtttagagtacacattaccaactggctttatcttgaaacaaaagattatggctactgagatgctacgtgttcgcactatgttgatgggtgatatccgtatgtccttacaagtagatactgacgtagtagatgaaacagcaatgagtggagcaagtgcaccaaactttgtacacggtcacgatgcaagccacttaatcttgacagtatgtgacttagtagataagggaatcaaatcaatagcagttattcatgattcatttggcacacatgcagataacacagtagctctacgtgactctttacgccataaaatggtggagatgtacgagaacaccaatgtattacagaagttactagaagagcatgaggagagatggctagttgatacaggtatcaaagttccagagcaaggcacttttgatataagagaaatcttaaactcagattatgtattcgcttaaggtattagtgacctgtaacagagcattagcgc

SEQ ID NO: 41 - DNA, 2837 bp, synthetic construct
  misc_feature 1..2837: note = Synthetic: F3675, RNApol157 Screening template (no terminator) - AT TSS
  source 1..2837: mol_type = other DNA
agatctcgatcccgcgaaatttgattaattaacccacactataatgagagaggggcacaacggtttcactctagaaaattaggggctactatagaagagagaaacatttacggcgaaggaggaaaaaatatgcatcatcatcaccatcacggcagcgctaacgtaatcaaaccagaatctcacaacttctcagatatctctgctgcaatcctaccgttcaacgtacttgcagactcttacggtgaggctctggccgctgaacagttgatgcttgagcatgaatcttaccagttaggtgaagcacgattcatcaaagcgatggaacgtcaagtagaacgtggtgaggtgtctgataatgctgttgctaaaccactgttagacactctaattcctgctctggcagctcgtatcactgagtttatagagatgaaacagcgcggtaaacctcacgtatcaaaaggttacttcgctatgattaagcctgagtctgctgcttttatcatcgttaagactactctgaatatccttgctaaagaagagtctgttccggttcaacgtgtagcaatggctatcggtggtaacatcgaagatgagattcgctttggtcgtatccgtgatgaagaaatcaaacacttcaaggagcgcgttaagcctaacttagacaagcgcaacgggtttatctacaagaaagcatacatggaagctgttgaagctggtatgcaagataaaggcgagttaaactctacgcatgaagcttgggaaaaagacgttaagtttcacgttggtatccgtgctattgaaatgcttatcgaagctactggcatggttcaacttgagcgtaagttcaaaggtatcccagataaagaccatgaagctttacaccttgcgcctgaatacgtagagaaactgactaaccgtgctcacgctctggcaggtatctcaccgatgtatcaaccaatgattgtcaaacctaagcgttggactggtgtacaaggtggtggctattgggctaaaggtcgtcgtccactgaacctaatccgtgtaggctctaagcgtgctctcgaccgttatcgtcaagtggatatgcctgaagtttacgatgcgattaacactattcaggagaccgcatggcgcattaacaaagatgttcttgctgtagttaacaacgtggtcacttgggctaactgtccggtagaagacgttccgtcaatcgacaagcttgctctacctgagaagcctgaagatatcgacaacaacgaggagtcactgaagaaatggaagaaggctgctgctgcaatctatcgtaaagagaaggctcgccaatctcgtcgaatcagcttagagtttgctttgagtcaagctaataagttcagcaagtacaacgagatttacttcccatacaacatggactggcgtggtcgcgtgtatgcaatcccgatgttcaatccacaaggtaacgatatggtgaaaggcttactgaccttcgctaagaaagttcctgtaggtatcgacggtggttactggttggctgttcatggtgcgaactgtgcaggcgttgacaaagtatcgcttgaagaccgtgttaaatgggtgaacgataacgaagctaatatcattgcaagcgctgaggctcctttagacttcacttggtgggctgagcaagactctcctttctgcttccttgcgttctgcttcgagtgggctgcgtatgtgaaagcaggtaagaaaccaagctttgagtcaagcttaccgctggccttcgatggtacttgctctggtctacaacacttctcagcgatgttacgtgatgaaatcggtggagctgctgttaacctgctacctgctgataaaccacaagatatctatggtatcgttgctgtgaaagtgaatgaggtactacgtgaccttgtgattagcggtacagaagacgagatgcaaactctcgaagacaagaagactggtgaaatcactgaacgtcttgtgcttggtactcgcactctggctgctcaatggttagaacatggtgtgactcgctcagtaactaaacgctcagttatgactctggcttatggttctaaggagtatggcttcgctgaccaagtgtttgaggacactgtgatgccagctatcgacaacggcaaaggtgctatgttcactgagccttcacaagcttgtcgtttcatggcgaaactcatttgggatgctgtaagtaaaactgtggttgctgctgtagaagctatgcaatggttacaatcagccgctaaactggtgtcttctgaggtgaaagacaagaagtcaggtgagattctgaaacatgcaatgcctgtacactggacgactcctaacggtttccctgtatggtctgagtattgcaaacaagaacagaagcgtattgattgcgttatcctcggtactcatcgcatggcgttaaccattaacatccgcgataagaaagagattgacgctgcaaaacagacttcaggtatcgctcctaacttcgttcacagtatggatgcgagtcacctacaaatgacagtcaacaagtgcttcaaagtgtatggtattcactccttcgctatgattcacgacagttttggttgccatgctggttttgcttctaagatgttccgtgcagtacgtgaaacaatggtagagacttatgaagaacatgatgtaatccaagagttctacaatcagtttgagcaacaacttcacgaatctcaaattgaaaagataccagttctaccacgcaaaggtaacttggagctgagagaaatcctcaagtctctatacactttctcttaaggtattagtgacctgtaacagagcattagcgc

SEQ ID NO: 42 - DNA, 6250 bp, synthetic construct
  misc_feature 1..6250: note = Synthetic: PP0881, Expression plasmid for RNApol137
  source 1..6250: mol_type = other DNA
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagctacactagcgatgagctttaccagaaacaacttgagctagaaggggagatgcatggctacggtgttactcgctttgaccgtaacaaccaacgtgcgattgatagcggtacaccttcagacactgattggaaccgccgtttactttctaacttcattgagcctatggtgaagggtattaatgcttataaggagtactacaagactaaagctggacggcctacagtggctttaaagtacatccggcaggtgcagccagagcaagcggcatacatcgccattaagaacattctggatgtgctgggtagtacaacatttgatgctaactggcttgttaccactattgggcgtaggattgaagaccaagtaagatttactaagcttgaggaggctgcacctaagtatgtaggcaaggttaaggatagcttagctaagcgcaactctctccagtatgcacaccagcataaggtactggtcgctactgagaagaagctagcggagaatccagaccgtgctcagcttgagctgccacgctggcaggagtgggctgaggaggattgcaagcacgttggtagcttgctggtcaatatctttgagcagtgtattctgtttgagggtgagccagtaatccgcaaggaggtgcagacagttcgcaaaggtacactggtcttcatcaagcctactgagaaagttaccaagtggattcatgagttcagagaggctattggcgggcttgctccagcttacgcaccttgcgtagttcctccgcttgattggacttccccatttactggcgggttccatactgaagctgttagcagcactctacatcttgctaaggttcgcaacaagcgccacctgcgcaagctgactaaagagcagatgccagcagtctacaaggctgtaaataacttgcagaaagtgcgctggcgtatctctgagcgtgtactggcaaccgctaatacactagtggagctgggcttgccttatgcgctgccgtcaaaggatgaaagcgactggaaagaaaagaatccatgccctgtacctgagtacctgcaagacctgcgaggcgagcagctgaaagcggcgcttactgccagccagtgggaggcttttcaagagtggaaacaactggcacgccagaactacgacgaggagagcgagcgcgtagcaagctttagggaagttgttcgtactttagggcaggctaaccgctacgttggctttgatgcaatctactttgtgtacacgctggactttcgcgggcgtgtctactgccagagcagccttgtaagcccacagggtggcgacttgcagaaagccctcatcaagtttgcggatggtatgaagctgggtgagcgcggcgagtactggttcaaggtgcatggcgctaacgaatggggctgggataagaagacctttgatgagcgcgtagctctggtcagcgaacctgagttctgcgagatgtgcctagacattgccagtgacccagtgaccttcaatgactggattaaggccgataagccgtggcagttcctcaactggtgctttgagtatgctgacttcctgaagcatgtgcaatcagctggcaacccacaggactttgtaagctatatcccttgtgcaatggatggttcttgctctggcatccagcactactctgcaatgctgcgcgatactgttggcggtactgctgttaacttggttaactctgacaagcctaatgacatctacggagaggtctgcaaggtgactattaaagagctgcaagcaattgctgacggctcacaggtatatgacagcaagattgaccctgtattagctcagcagctggcgcaggagtggctacgtctgcaaccaaatcgcagcctgaccaagaaacctgttatgaccctgccttacggcagcactcagttaacctgccgtgagcatgttagccagtggcttaaagatttgcagaaggaggagaacaagcgagctaaggcagagttcagagagcctatgaaggtacacgctttcggcgatgcagacagcgctatgccgctaaagtttgctgagtctcttatgactagcattgtttggcactctatcggcaaggttgtagtagctgcgagggctggtatggcttacatcaaagccgtaacaagctcagtggctaagatgaatatgccgcttgagtggactactccaacaggctttattgtccgtcaggagatttaccagttcacaacacgtcaggtaaatactcagctgctgggtggaactaagtttgtggtcagcactaagagtaaagatattgattatcatcgtatgattaactcctgtgcgcctaactttgttcactcaatggatgcgagccaccttacactggcaactaactactttgcagatgcaggtattagcagcattgcagtaatccatgatagctttggcacacatgcaggagccactgacctgctgcgtgagcgtctgcgagcttcaatggtagatatgtacgaacagcatgacgttatcactaacttccttgctgaaactgagggcagacttatgacagcttttgagcacattcgagtgccagagcgtggtgagctgcaacttgatagcattaatcaatctacatacgcttttgcgtgatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 43 - DNA, 6244 bp, synthetic construct
  misc_feature 1..6244: note = Synthetic: PP0891, Expression plasmid for RNApol136
  source 1..6244: mol_type = other DNA
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagccaagatttacacgctattcaactgcaatttgaagaagagatgttcaacggcggcattcgtcgcttcgaggctgacaatcagcgtgttatagccagcggtaacgagtcagaaaccgcatggaatcgccgtttactctctgaattgatcgcacctatggctgaaggcatcaacgcctataaagaatcttacgttggcaagcgtggaaaacctgctttagcccttgccttccttcagtgcgtagagaatgaagttgcagcatatatcacaatgaaggttgttatggatatgctcaacactgacgtaaccctgcaagccgttgcgatgaccattgctgagcgtattgaagatcaggtacgcttctctaagctggatgcacacgccaacaaatactttgagaaggttaaggccagcctcaaggcgtcaaaatctaagcaataccgccacgggcaccgcgtaatggtagccgctgagaaatcagttagtgagaaagatgccgattttgaccgctgggaagcatggcctaaagaaacttgcttgcaaatcggggctaccttgctggatattctggaatctagtgtgttctatcagggtgagcctgtattcttccgcgctatccgtcaaaatggcgtgcgctctacgtactaccttcagacttctgagactgtgggggcatggattgaggagtttaaagagcacgtggcacagcttgccccagcttatgccccttgtgttgtccctccccgtgactggaaaagcccgttcaatggtggattccatactgagaaggtatcaagccgtgtgcgtctggttaaaggtgcgcgtgagcatgtacgcaagttgaccgttaagcagatgccgaatgtgtacaaggccatcaacgcattgcagcgcactgaatggcaagttaacactgacgttatgaaagtcgctgatgacgttatccgccttaacctaggctatgggatgccgtcctttaagcctcttatcgataaagagaacaagccacttaaccctgtgcctgtggagtttcagcatttgcgcgggcgtgagttgaaagaaatgctcaccgctgagcaatgggatacgtttatcgcatggaaaggtgagtgctcacgcctgtataccgctgaaactaagcgaggctctaagagcgccgccgtggttcgcatggtgggacaagcccgtaaatactccgcgtttaacgccatccattttgtttacgcgatggattcccgcagccgtgtatatgcgcaatcctccacgctgtcaccgcaatccaatgacttaggcaaggcattgctccgctttactgagaaacgcgccctaaatggctctcaggcgctcaaatggttctgtgtcgcgggcgctaacctttggggctgggacaagaaaaccttcggcgtgcgcgtgtccaacgtgttagacggcgagtttcaagatatgtgtcgtgatatcgccgctgacccgctcacctttacacagtgggctggcgcggacgagccttatcagttcctagcgtgggcgatggaatacgcgaactaccttgacctgttagatgaagaccgtcaagaagagttccgtacacagttgccagttcatcaggacggctcttgctcaggcatccagcactattccgcaatgctgaaggacagcgtaggcgcggcggcggtcaacctcctgcctagcgacgaaccacaggatatctacgggcgggtagctcaggtagttatcggcaaggtggcacagcacgctaacgcactggacgcggatacgttcacttctgggaagctgacactcacaggcgatgcactgcgcaccatggcggcatcgtgggacgcaataggtatcacgcgcggtctgactaaaaagccagtcatgacactgccgtatggctcaacccgtatcacctgccgtgaagctgtagccgattacctgattgacctagaagagaaagaggctcaaaaggcggtggcagaagggcgaggagtgaaccttgtgcacccattcggacaggcagaaggacagatgaccgaagcgatggccttgaactacatgacggcgcttatctggccttccatttctgaagttgttaaggctccaatcgtcgcaatgaagatgatccgtgcgctggctcgctttgctgcgaaacgaaatgaagggctggagtatacgctccctactggcttcattcttcagcaaaagattatggcgaccgaccttctgcgcgtgcgcactgtgttgagcggcgatataaaaatggctctgcgactgcaagtcgataccgatattgtagacgaatcagcgatgatgggtgctgcggctcctaactttgttcacggacacgatgcgagccacttgattctgtccgtctgcgcaatggtggacgaagggattacctcaattgcagtcattcacgactctttcggtactcacgcagaccacacaccagacctccgcaacgcactgaaagggcagatggttgagatgtacgccaacactaacgcgctccagaaactgctgagcgaacacgaagaccgctggatggtagatactaagattgaagtaccggagcaaggtgactttgacgtaaacctgattatggagtcggaatactgcttcgcataatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 44 - DNA, 6028 bp, synthetic construct
  misc_feature 1..6028: note = Synthetic: PP0856, Expression plasmid for RNApol031
  source 1..6028: mol_type = other DNA
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcaatcaggcggaacttgagcgggaaatggcagacggcggtcgccgtgcggcaatggcaaggtttgaaaccgccgagtccgcgaacgacgcagcgagtaacccatatgccgccgcgatcttccgccgattcatcaagcctctcagcgaggggcttgacgtgtaccttcacaaagcagtccgaggcgtagcagcgaaatcaaaggtgctgctgcgcggacaggacacgctggcgttagcgtacatcacgatccgtggtgtgattaacgcaagcctctccgaaggggaggcagtatttgctggtgtggccatggagattggccgcactgtgtacagcgaggtggctctcaggcaattcgaagacctgaacccggagctgtattacacgctggtgcaagaccttgaacgccgcatgaccaagagcgagcgtcaccggttcaacgtgatgcgatccagtgccgagaaggacggaacgccgttgccggtgtgggagccagcgctcaagatggacgtgggtacgttgttgctgggcgcaagccttgacatcgggctgatcgaaacctttgatctgcgcatgaagggcaagacaaccaagcacttgaggctgcacccggatctgctggagctgatcgagcagattaaggggttcgtggccggtgcttcgccgttcaatctgccgtgcgtcgagccgccgaaaccatgggttacgcctatggacggtggctggcacacgaatgcgatgcgccggacgctgccttgcatggtacgcggacaggcatcggcaacgattgaggacgtaggcccgcgagttctcaaggcactgaacaaggtgcagagcgttgcgtgggaggtgaacgagcgaattcttgaggtagcggagttcgcaagggagcattttgacgtacaggacgtgctggttagcgataagcgcggcgggatgccggacaagcctctgtttatgcaggctgatcccgacctcaaaatcgttgacatgaacgaattcgaactggcacagttcactgaatggcgacaggaggcacgagaatggcacactcagcagaaggtacggggagcgcatgcggggagtacgaacgaagctatccgcgtggcgaacaagtacaaggggaacccgctctggtttgtctattctgtggactatcggagccgattctatgcctctggacaggggctctccccacaagggaacgacctttcaaaagcgctcatccactttgcaaagtctgcacctatacgaacttccgctggcctcttctggttccgggtagccggagctaacaagtgggccgttgacaaactagacaagaagccgctcgatgttcgggcgcaatgggtgatagacaatgaagagtttatcagtcggatcgctgatgacccgatcagccaccgagaatggacagatgctgacgtacctttccagtttcttgcgtggtgctttgaatatgcagcttggcggaaagaccctgaatcgtttcgcactcgaatcccacttggacaagacggcagttgtaatggtcttcaacacttctcagcaatgcttagagatagggttggcgggcgagcaaccaacctcattcccgataccgtgcaacacgacatctatggcctcgtcgcaacagcaactgctggcatcgtgcaagcggacattgacgaatgcgatattgccaagcgctggaagtcacacgaactttcgcggtcgttggttaagcggtcagtaatgaccctgccgtatggctccacacggcactcctgccgcgacttcatcatgaaggagtacatggacaaggggagtgcgccggagttcgacaagcgcgagaatgagcctgctgcgcggtggttgtcgtaccgggtatgggacggcatcggtgaggtggtcgtgaagggccgacaggcgatggagtggttgcaggcagcatcggccatcatgtgcgccgggaacgccccacacatcgaatggaggaaccctgcgggctttctggtacggcagcgttaccacgcccgggaaatgctcagggtgagctgtcacagcctctcaggcaagcgtatccgcatcaacgtccagaccttccgggacgagggcgatccacggcggcaccggaacggcatcgcgccgaacttcgtgcatagctgtgacgcggctcacctgacgttcttcacgaacgactgggaggacgaggacggcggcgatctggcactcgtccacgatgactacggcgcactggccgacgaggtgcccaagctgcacagaatcctgcgtaggtcgtttgtggacatgtacctgtaccatgaccctctgaaagcgcttgcggcgcaggtggagggcttaccggagcttcctgagtccggggatctagatcttgaggaagtaaaccggtcggtgtacttcttctgttaatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 45 - DNA, 27 bp, synthetic construct
  misc_feature 1..27: note = Synthetic: PG5878, RNApol002, insert #1, forward primer
  source 1..27: mol_type = other DNA
agatctcgatcccgcgaaattcgaccc

SEQ ID NO: 46 - DNA, 30 bp, synthetic construct
  misc_feature 1..30: note = Synthetic: PG5877, RNApol031, insert #1, forward primer
  source 1..30: mol_type = other DNA
agatctcgatcccgcgaaatattgggccac

SEQ ID NO: 47 - DNA, 32 bp, synthetic construct
  misc_feature 1..32: note = Synthetic: PG5879, RNApol126, insert #1, forward primer
  source 1..32: mol_type = other DNA
agatctcgatcccgcgaaattaattagagacc

SEQ ID NO: 48 - DNA, 30 bp, synthetic construct
  misc_feature 1..30: note = Synthetic: PG5881, RNApol136, insert #1, forward primer
  source 1..30: mol_type = other DNA
agatctcgatcccgcgaaataattaggggc

SEQ ID NO: 49 - DNA, 31 bp, synthetic construct
  misc_feature 1..31: note = Synthetic: PG5880, RNApol161, insert #1, forward primer
  source 1..31: mol_type = other DNA
agatctcgatcccgcgaaataattaacccac

SEQ ID NO: 50 - DNA, 48 bp, synthetic construct
  misc_feature 1..48: note = Synthetic: PG5884, RNApol002, insert #1, reverse primer
  source 1..48: mol_type = other DNA
tgctaactgctgttccagcgtaatgctgccgtgatggtgatgatgatg

SEQ ID NO: 51 - DNA, 40 bp, synthetic construct
  misc_feature 1..40: note = Synthetic: PG5827, RNApol002, insert #2, reverse primer
  source 1..40: mol_type = other DNA
gagttggatcctcacgtgcattagcggaagaaattcggtg

SEQ ID NO: 52 - DNA, 40 bp, synthetic construct
  misc_feature 1..40: note = Synthetic: PG5539, RNApol031, insert #1, reverse primer
  source 1..40: mol_type = other DNA
cgctcaagttccgcctgattgctgccgtgatggtgatgat

SEQ ID NO: 53 - DNA, 40 bp, synthetic construct
  misc_feature 1..40: note = Synthetic: PG5003, RNApol031, insert #2, reverse primer
  source 1..40: mol_type = other DNA
gagttggatcctcacgtgcattaacagaagaagtacaccg

SEQ ID NO: 54 - DNA, 48 bp, synthetic construct
  misc_feature 1..48: note = Synthetic: PG5887, RNApol126, insert #1, reverse primer
  source 1..48: mol_type = other DNA
ctgtaattgaatttcttgtaaatcgctgccgtgatggtgatgatgatg

SEQ ID NO: 55 - DNA, 40 bp, synthetic construct
  misc_feature 1..40: note = Synthetic: PG5828, RNApol126, insert #2, reverse primer
  source 1..40: mol_type = other DNA
gagttggatcctcacgtgcattaagcgaatacataatctg

SEQ ID NO: 56 - DNA, 48 bp, synthetic construct
  misc_feature 1..48: note = Synthetic: PG5891, RNApol136, insert #1, reverse primer
  source 1..48: mol_type = other DNA
cagttgaatagcgtgtaaatcttggctgccgtgatggtgatgatgatg

SEQ ID NO: 57 - DNA, 40 bp, synthetic construct
  misc_feature 1..40: note = Synthetic: PG5830, RNApol136, insert #2, reverse primer
  source 1..40: mol_type = other DNA
gagttggatcctcacgtgcattatgcgaagcagtattccg

SEQ ID NO: 58 - DNA, 48 bp, synthetic construct
  misc_feature 1..48: note = Synthetic: PG5531, RNApol137, insert #1, reverse primer
  source 1..48: mol_type = other DNA
ctggtaaagctcatcgctagtgtagctgccgtgatggtgatgatgatg

SEQ ID NO: 59 - DNA, 48 bp, synthetic construct
  misc_feature 1..48: note = Synthetic: PG5889, RNApol161, insert #1, reverse primer
  source 1..48: mol_type = other DNA
agattctggtttgattacgttagcgctgccgtgatggtgatgatgatg

SEQ ID NO: 60 - DNA, 40 bp, synthetic construct
  misc_feature 1..40: note = Synthetic: PG5829, RNApol161, insert #2, reverse primer
  source 1..40: mol_type = other DNA
gagttggatcctcacgtgcattaagagaaagtgtatagag

SEQ ID NO: 61 - DNA, 78 bp, synthetic construct
  misc_feature 1..78: note = Synthetic: PG6178, forward primer for regeneration from cDNA (RNApol031)
  modified_base 73..76: mod_base = OTHER; note = phosphorothioate linkages
  source 1..78: mol_type = other DNA
agatctcgatcccgcgaaatattgggccactatagcaacaccaaaaccatcatcacaacggtttcactctagaatagg

SEQ ID NO: 62 - DNA, 71 bp, synthetic construct
  misc_feature 1..71: note = Synthetic: PG6179, forward primer for regeneration from cDNA (RNApol002)
  modified_base 66..69: mod_base = OTHER; note = phosphorothioate linkages
  source 1..71: mol_type = other DNA
agatctcgatcccgcgaaattcgaccctattaaacaaggagccaattacaacggtttcactctagaaattg

SEQ ID NO: 63 - DNA, 91 bp, synthetic construct
  misc_feature 1..91: note = Synthetic: PG6180, forward primer for regeneration from cDNA (RNApol126)
  modified_base 86..89: mod_base = OTHER; note = phosphorothioate linkages
  source 1..91: mol_type = other DNA
agatctcgatcccgcgaaattaattagagaccactatagaagagataaattttaatggcaattcagcacaacggtttcactctagaaattg

SEQ ID NO: 64 - DNA, 83 bp, synthetic construct
  misc_feature 1..83: note = Synthetic: PG6181, forward primer for regeneration from cDNA (RNApol161)
  modified_base 78..81: mod_base = OTHER; note = phosphorothioate linkages
  source 1..83: mol_type = other DNA
agatctcgatcccgcgaaataattaacccacactatagggctacttccaacaaccacatacaacggtttcactctagaaattg

SEQ ID NO: 65 - DNA, 83 bp, synthetic construct
  misc_feature 1..83: note = Synthetic: PG6182, forward primer for regeneration from cDNA (RNApol136)
  modified_base 78..81: mod_base = OTHER; note = phosphorothioate linkages
  source 1..83: mol_type = other DNA
agatctcgatcccgcgaaataattaggggctactatagaagagagaaacatttacggcgacaacggtttcactctagaaattg

SEQ ID NO: 66 - DNA, 48 bp, synthetic construct
  misc_feature 1..48: note = Synthetic: PG5335, PCR primer to create a linear amplicon from plasmid PP0396
  source 1..48: mol_type = other DNA
gctgccgtgatggtgatgatgatgcatggttaattcctcctgttagcc

SEQ ID NO: 67 - DNA, 22 bp, synthetic construct
  misc_feature 1..22: note = Synthetic: PG5772, Other qPCR primers
  source 1..22: mol_type = other DNA
ctaatgccctgatcgacaaaga

SEQ ID NO: 68 - DNA, 21 bp, synthetic construct
  misc_feature 1..21: note = Synthetic: PG5773, Other qPCR primers
  source 1..21: mol_type = other DNA
cttcagccggtcaactatgaa

SEQ ID NO: 69 - protein, 811 aa, synthetic construct
  REGION 1..811: note = Synthetic: RNApol031
MHHHHHHGSNQAELEREMADGGRRAAMARFETAESANDAASNPYAAAIFRRFIKPLSEGLDVYLHKAVRGVAAKSKVLLRGQDTLALAYITIRGVINASLSEGEAVFAGVAMEIGRTVYSEVALRQFEDLNPELYYTLVQDLERRMTKSERHRFNVMRSSAEKDGTPLPVWEPALKMDVGTLLLGASLDIGLIETFDLRMKGKTTKHLRLHPDLLELIEQIKGFVAGASPFNLPCVEPPKPWVTPMDGGWHTNAMRRTLPCMVRGQASATIEDVGPRVLKALNKVQSVAWEVNERILEVAEFAREHFDVQDVLVSDKRGGMPDKPLFMQADPDLKIVDMNEFELAQFTEWRQEAREWHTQQKVRGAHAGSTNEAIRVANKYKGNPLWFVYSVDYRSRFYASGQGLSPQGNDLSKALIHFAKSAPIRTSAGLFWFRVAGANKWAVDKLDKKPLDVRAQWVIDNEEFISRIADDPISHREWTDADVPFQFLAWCFEYAAWRKDPESFRTRIPLGQDGSCNGLQHFSAMLRDRVGGRATNLIPDTVQHDIYGLVATATAGIVQADIDECDIAKRWKSHELSRSLVKRSVMTLPYGSTRHSCRDFIMKEYMDKGSAPEFDKRENEPAARWLSYRVWDGIGEVVVKGRQAMEWLQAASAIMCAGNAPHIEWRNPAGFLVRQRYHAREMLRVSCHSLSGKRIRINVQTFRDEGDPRRHRNGIAPNFVHSCDAAHLTFFTNDWEDEDGGDLALVHDDYGALADEVPKLHRILRRSFVDMYLYHDPLKALAAQVEGLPELPESGDLDLEEVNRSVYFFC

SEQ ID NO: 70 - protein, 883 aa, synthetic construct
  REGION 1..883: note = Synthetic: RNApol136
MHHHHHHGSQDLHAIQLQFEEEMFNGGIRRFEADNQRVIASGNESETAWNRRLLSELIAPMAEGINAYKESYVGKRGKPALALAFLQCVENEVAAYITMKVVMDMLNTDVTLQAVAMTIAERIEDQVRFSKLDAHANKYFEKVKASLKASKSKQYRHGHRVMVAAEKSVSEKDADFDRWEAWPKETCLQIGATLLDILESSVFYQGEPVFFRAIRQNGVRSTYYLQTSETVGAWIEEFKEHVAQLAPAYAPCVVPPRDWKSPFNGGFHTEKVSSRVRLVKGAREHVRKLTVKQMPNVYKAINALQRTEWQVNTDVMKVADDVIRLNLGYGMPSFKPLIDKENKPLNPVPVEFQHLRGRELKEMLTAEQWDTFIAWKGECSRLYTAETKRGSKSAAVVRMVGQARKYSAFNAIHFVYAMDSRSRVYAQSSTLSPQSNDLGKALLRFTEKRALNGSQALKWFCVAGANLWGWDKKTFGVRVSNVLDGEFQDMCRDIAADPLTFTQWAGADEPYQFLAWAMEYANYLDLLDEDRQEEFRTQLPVHQDGSCSGIQHYSAMLKDSVGAAAVNLLPSDEPQDIYGRVAQVVIGKVAQHANALDADTFTSGKLTLTGDALRTMAASWDAIGITRGLTKKPVMTLPYGSTRITCREAVADYLIDLEEKEAQKAVAEGRGVNLVHPFGQAEGQMTEAMALNYMTALIWPSISEVVKAPIVAMKMIRALARFAAKRNEGLEYTLPTGFILQQKIMATDLLRVRTVLSGDIKMALRLQVDTDIVDESAMMGAAAPNFVHGHDASHLILSVCAMVDEGITSIAVIHDSFGTHADHTPDLRNALKGQMVEMYANTNALQKLLSEHEDRWMVDTKIEVPEQGDFDVNLIMESEYCFA

SEQ ID NO: 71 - protein, 885 aa, synthetic construct
  REGION 1..885: note = Synthetic: RNApol137
MHHHHHHGSYTSDELYQKQLELEGEMHGYGVTRFDRNNQRAIDSGTPSDTDWNRRLLSNFIEPMVKGINAYKEYYKTKAGRPTVALKYIRQVQPEQAAYIAIKNILDVLGSTTFDANWLVTTIGRRIEDQVRFTKLEEAAPKYVGKVKDSLAKRNSLQYAHQHKVLVATEKKLAENPDRAQLELPRWQEWAEEDCKHVGSLLVNIFEQCILFEGEPVIRKEVQTVRKGTLVFIKPTEKVTKWIHEFREAIGGLAPAYAPCVVPPLDWTSPFTGGFHTEAVSSTLHLAKVRNKRHLRKLTKEQMPAVYKAVNNLQKVRWRISERVLATANTLVELGLPYALPSKDESDWKEKNPCPVPEYLQDLRGEQLKAALTASQWEAFQEWKQLARQNYDEESERVASFREVVRTLGQANRYVGFDAIYFVYTLDFRGRVYCQSSLVSPQGGDLQKALIKFADGMKLGERGEYWFKVHGANEWGWDKKTFDERVALVSEPEFCEMCLDIASDPVTFNDWIKADKPWQFLNWCFEYADFLKHVQSAGNPQDFVSYIPCAMDGSCSGIQHYSAMLRDTVGGTAVNLVNSDKPNDIYGEVCKVTIKELQAIADGSQVYDSKIDPVLAQQLAQEWLRLQPNRSLTKKPVMTLPYGSTQLTCREHVSQWLKDLQKEENKRAKAEFREPMKVHAFGDADSAMPLKFAESLMTSIVWHSIGKVVVAARAGMAYIKAVTSSVAKMNMPLEWTTPTGFIVRQEIYQFTTRQVNTQLLGGTKFVVSTKSKDIDYHRMINSCAPNFVHSMDASHLTLATNYFADAGISSIAVIHDSFGTHAGATDLLRERLRASMVDMYEQHDVITNFLAETEGRLMTAFEHIRVPERGELQLDSINQSTYAFA

SEQ ID NO: 72 - DNA, 2937 bp, synthetic construct
  misc_feature 1..2937: note = Synthetic: 5' UTR screening template
  source 1..2937: mol_type = other DNA
atccttaataattaggcactacaataggagaaccacaacagtaacaacggtttcactctagataattaggggctactatagaagagagatcacatttgcttctgacatagttgtgttgactcacaaccccagaaacagacatcgccaccatgtacaccagcgacgagctgtaccagaagcagctggaactggaaggcgagatgcacggctacggcgtgaccagattcgaccggaacaaccagagagccatcgacagcggcacccctagcgacaccgattggaatagacggctgctgagcaacttcatcgagcccatggtcaagggcatcaacgcctacaaagagtactacaagaccaaggccggcagacccacagtggccctgaagtacattagacaggtgcagcctgagcaggccgcctatatcgccatcaagaacatcctggacgtgctgggcagcaccaccttcgatgctaattggctggtcaccaccatcggcagacggatcgaagatcaagtgcggttcaccaagctggaagaggccgctcctaaatacgtgggcaaagtgaaggacagcctggccaagagaaacagcctgcagtatgcccaccagcacaaggtgctggtcgccaccgaaaagaagctggccgagaatcccgatagagcccagctcgaactgcccagatggcaagaatgggccgaagaggactgcaagcacgtgggaagcctgctggtcaacatcttcgagcagtgcatcctgttcgagggcgagcccgtgatcagaaaagaggtgcagaccgtcagaaagggcaccctggtgttcatcaagcccaccgagaaagtgaccaagtggattcacgagttccgcgaggccatcggaggactggctccagcttatgctccttgcgttgtgcctcctctggactggacaagccctttcacaggcggctttcacaccgaggccgtgtctagcacactgcacctggcaaaagtgcggaacaagcggcacctgaggaagctgaccaaagaacagatgcccgccgtgtacaaggccgtgaacaacctgcagaaagtccggtggcggatcagcgaaagagtgctggccacagccaacacactggtggaactgggcctgccttacgctctgcctagcaaggatgagagcgactggaaagagaagaacccctgtcctgtgcctgagtacctgcaggatctgagaggcgagcagctgaaggctgccctgacagcttctcagtgggaagctttccaagagtggaaacagctggcccggcagaactacgacgaggaatctgaaagagtggcctccttcagagaagtcgtgcggacactcggccaggccaacagatatgtgggcttcgacgccatctacttcgtgtacaccctggacttccggggcagagtgtactgtcagtccagtctggtgtctccccaaggcggcgatctgcagaaggccctgatcaagtttgccgacggcatgaagctgggcgaaagaggcgagtactggttcaaagtgcacggcgccaatgagtggggctgggacaagaaaaccttcgacgagagagtggccctcgtcagcgagcctgagttctgcgagatgtgcctggatatcgccagcgatcccgtgaccttcaacgactggatcaaggccgacaagccttggcagttcctgaactggtgcttcgagtacgccgatttcctgaagcacgtgcagagcgccggcaatccccaggacttcgtgtcttacatcccctgcgccatggatggcagctgttctggcatccagcactacagcgccatgctgagagataccgttggcggcacagccgtgaacctcgtgaacagcgacaagcccaacgacatctacggcgaagtgtgcaaagtgacgatcaaagagctgcaggctatcgccgatggcagccaggtgtacgacagcaagatcgatcctgtgctggctcagcaactggcccaagaatggctgagactgcagcccaaccgcagcctgaccaaaaagcctgtgatgaccctgccatacggcagcacccaactgacctgcagagaacacgtttcccagtggctgaaggacctccagaaagaggaaaacaagcgcgccaaggccgagttcagagaacccatgaaggtgcacgccttcggcgacgccgattctgccatgcctctgaagttcgccgagagcctgatgaccagcatcgtgtggcacagcattggcaaggtggtggtggctgccagagccggcatggcctatatcaaagccgtgacaagctccgtggccaagatgaacatgcccctggaatggaccacacctaccggcttcattgtgcggcaagagatctaccagttcaccacacggcaagtgaacacacagctgcttggcgggaccaagttcgtggtgtccaccaagagcaaggacatcgactaccaccggatgatcaacagctgcgcccctaacttcgtgcacagcatggacgcctctcacctgacactggccaccaactactttgctgacgccggcatctcctctatcgccgtgatccacgatagcttcggcacacatgctggcgccacagacctgctgagggaaagactgagagccagcatggtggatatgtacgagcagcacgatgtgatcaccaactttctggccgaaaccgagggcagactgatgaccgccttcgagcacatcagagtgcccgagagaggcgaactgcagctggacagcatcaaccagagcacctacgccttcgcctgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaa

SEQ ID NO: 73 - DNA, 164 bp, synthetic construct
  misc_feature 1..126: note = Synthetic: Randomized PCR primer encoding degenerate 5' UTR
  source 1..164: mol_type = other DNA
atccttaataattaggcactacaataggagaaccacaacagtaacaacggtttcactctagataattaggggctactatagaagagagatcacatttgcttctnnnnnnnnnnnnnnnnnnnnnnnnnnnnnnnnnnnnnnnngccaccatgtacaccagcgac

SEQ ID NO: 74 - DNA, 59 bp, synthetic construct
  misc_feature 1..59: note = Synthetic: Reverse primer for template amplification with polyA tail
  source 1..59: mol_type = other DNA
tttttttttttttttttttttgcaatgaaaataaatgttttttattaggcagaatccag

SEQ ID NO: 75 - DNA, 27 bp, synthetic construct
  misc_feature 1..27: note = Synthetic: Sanger sequencing primer
  source 1..27: mol_type = other DNA
gccaattagcatcgaaggtggtgctgc

SEQ ID NO: 76 - DNA, 27 bp, synthetic construct
  misc_feature 1..27: note = Synthetic: Reverse transcription primer
  source 1..27: mol_type = other DNA
gccttccagttccagctgcttctggta

SEQ ID NO: 77 - DNA, 49 bp, synthetic construct
  misc_feature 1..49: note = Synthetic: cDNA amplification primer 1
  source 1..49: mol_type = other DNA
atccttaataattaggcactacaataggagaaccacaacagtaacaacg

SEQ ID NO: 78 - DNA, 48 bp, synthetic construct
  misc_feature 1..48: note = Synthetic: cDNA amplification primer 2
  source 1..48: mol_type = other DNA
gccttccagttccagctgcttctggtacagctcgtcgctggtgtacat